(19) United States
(12) Patent Application Publication
(10) Pub. No.: US 2008/0095764 A1
(43) Pub. Date: Apr. 24, 2008

(54) Use of genetically-and epigenetically-altered protocadherins in methods of diagnosing, prognosing, and treating cancer

(75) Inventors: Ramon Parsons, Manhasset, NY (US); Jennifer Yu, San Francisco, CA (US); Satoru Nagase, Miyagi (JP)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/637,277

(22) Filed: Dec. 12, 2006

Related U.S. Application Data
(63) Continuation-in-part of application No. PCT/US05/21602, filed on Jun. 17, 2005.
(60) Provisional application No. 60/581,215, filed on Jun. 17, 2004.

(51) Int. Cl. A61K 31/70; A61K 38/00; A61K 39/395; A61P 43/00; C12Q 1/68; G01N 33/53

(52) U.S. Cl. 424/130.1; 435/6; 435/7.23; 435/7.9; 514/12; 514/44

(57) ABSTRACT

The present invention provides methods for determining whether a subject has neoplasia, for assessing the efficacy of therapy to treat neoplasia in a subject who has undergone or is undergoing treatment for neoplasia, and for assessing the prognosis of a subject who has neoplasia. The present invention further provides kits for use in detecting neoplasia. The present invention also provides methods for treating or preventing neoplasia. Additionally, the present invention provides pharmaceutical compositions for use in treating or preventing neoplasia in subjects to whom the compositions are administered. The present invention further provides methods for identifying agents for use in treating and/or preventing neoplasia. Also provided are agents identified by these methods, and use of these agents in methods for treating or preventing neoplasia.

This application is a continuation-in-part of International Application No. PCT/US2005/021602, filed Jun. 17, 2005, and claims priority to U.S. Provisional Application Ser. No. 60/581,215, filed Jun. 17, 2004; both applications are herein incorporated by reference in their entireties.

This invention was made with government support under NIH Grant No. R01 CA082783. As such, the United States government has certain rights in this invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### BACKGROUND OF THE INVENTION

Neoplasia is a disease characterized by an abnormal proliferation of cells, or neoplasm. In neoplasia, cells divide and grow uncontrollably, they invade and disrupt other tissues, and often spread to other areas of the body (metastasis). Neoplasms may manifest as a blood disorder (e.g. leukemia) or a tumor, and may be benign or malignant (cancer). Successful management of neoplasms depends, in part, upon early detection of tumors. A correlation generally exists between the tumor burden in a patient with a neoplasm, and the patient's chances of survival. Thus, the mortality from cancer can be reduced if tumors are found and treated at an early stage.

The major therapies for treating neoplasia are surgery and radiotherapy (for local and local/regional neoplasms) and chemotherapy (for systemic sites) (Beers and Berkow, Eds., The Merck Manual of Diagnosis and Therapy, 17th ed. (Whitehouse Station, N.J.: Merck Research Laboratories, 1999) 973-74, 976, 986, 988, 991). While treatment-related improvements in survival may have resulted in a decline in cancer mortality, current treatment protocols can be invasive (surgery) or can produce deleterious side effects (chemotherapy and radiation).

By way of example, breast cancer is the most common malignancy among women, and remains the leading cause of cancer-related death in women aged 20-59 (Greenlee et al., Cancer Statistics 2000. A Cancer Journal for Clinicians, 50:7-33, 2000). Breast examination, mammography, xerography, and termography are established methods of detecting malignant breast masses. These methods, in conjunction with personal history, may strongly suggest breast cancer, however, a true diagnosis can only be made by microscopic examination of tissue removed by excisional biopsy or aspiration. There are a number of methods currently used to treat breast cancer, including surgery, radiotherapy, hormone therapy, and chemotherapy. Because breast tumors may be cured with combined modality therapy, each of these methods may be used alone, or in addition to one or more other therapies. Thus, local and regional therapy, surgery, or radiotherapy is often integrated with systemic therapy (e.g. chemotherapy).

Despite the various methods for detecting, diagnosing, and treating neoplasias, including breast neoplasms, cancer remains prevalent in all segments of society, and is often fatal. Clearly, alternative strategies for detection (including the development of markers that can identify neoplasias at an early stage) and for treatment are needed to improve survival in cancer patients. In particular, a better understanding of tumor suppressors, and tumor-suppression pathways, would provide a basis from which novel detection, diagnostic, and treatment regimens may be developed.

Cadherins are functionally-related integral membrane glycoproteins that play an important role in calcium-dependent cell-cell adhesion. Cadherins promote cell adhesion via a homophilic mechanism, and play a role in the construction of tissues and of the animal body as a whole. There are three types of cadherin, each of which is distinct in its immunological specificity and tissue distribution: N cadherin is found in neural tissue, E cadherin (also known as uvomorulin or L CAM) is found in epithelial tissue, and P cadherin is found in placental tissue. Each cadherin protein has a 600-amino-acid extracellular domain, containing four repeats (believed to contain the calcium-binding sites), a transmembrane domain, and a one 50-amino-acid intracellular domain. Cadherin expression appears to be tightly regulated during development, with each tissue or cell type showing a characteristic pattern of cadherin molecules. Inappropriate regulation of cadherin expression levels or functionality has been observed in human malignancies, and has been shown to lead to aggravated cancer-cell invasion and metastasis.

There are at least six subfamilies in the cadherin superfamily: classical or type-I cadherins, atypical or type-II cadherins, desmocollins, desmogleins, protocadherins, and Flamingo cadherins. These subfamilies are distinguished on the basis of protein domain composition, genomic structure, and phylogenetic analysis of protein sequence. The protocadherins constitute a large subfamily of the cadherin superfamily, and function in a variety of tissues and multicellular organisms. Protocadherins are usually localized at synapses in the central nervous system (CNS), and their expression is spatiotemporally regulated.

A highly conserved protocadherin (PCDH) cluster exists at human chromosome 13q. This cluster contains PCDH8, PCDH9, PCDH17, and PCDH20. These protocadherins are single-pass transmembrane proteins that share greater similarity with each other than with other members of the protocadherin family. Like other protocadherins, the PCDHs in the 13q cluster have six cadherin repeats in the extracellular domain, a single transmembrane domain, a signal peptide sequence, and an intracellular domain; however, they have unique cytoplasmic tails. Interestingly, while the extracellular domain of PCDH8 is most similar to that of PCDH17, the intracellular domain of PCDH8 is most similar to that of PCDH9.

Protocadherin 8 and protocadherin 9 constitute a linkage group on human chromosome 13 and mouse chromosome 14. Like other protocadherins, PCDH8 and PCDH9 are predominantly expressed in the brain, and exhibit expression patterns that are developmentally regulated; PCDH9 is also expressed in a broader variety of tissues. In contrast to a classical or a desmosomal cadherin—which generally consists of 15-17 exons, and shares a remarkable degree of conservation in intron positions—PCDH8 has only three exons. The first exon encodes the extracellular domain, the transmembrane region, and part of the cytoplasmic tail; the second and third exons encode the remainder of the cytoplasmic region, including the 3′ untranslated region. Protocadherin 17, also known as PCDH68, encodes a protein which contains six extracellular cadherin domains, a transmembrane domain, and a cytoplasmic tail that differs from those of the classical cadherins.

Many protocadherins are believed to play in important role in the organization; of the CNS. Prior to the present invention, however, it was not known that PCDHs 8, 9, and 17 are also markers for, and form a basis for designing therapeutics to treat, neoplasia.

### SUMMARY OF THE INVENTION

The present invention is based upon the surprising discovery that human protocadherins on chromosome 13q 14-21 are activated or inactivated, as the case may be, in an oncogenic switch. For example, the inventors have discovered that protocadherin 17 is turned on, and protocadherins 8 and 9 are shut off, in cancer.

Accordingly, in one aspect, the present invention provides a method for determining whether a subject has neoplasia, by assaying a diagnostic sample of the subject for expression of at least one protocadherin selected from the group consisting of PCDH8, PCDH9, and PCDH17, wherein detection of PCDH8 expression decreased below normal is diagnostic of neoplasia in the subject, detection of PCDH9 expression decreased below normal is diagnostic of neoplasia in the subject, and detection of PCDH17 expression elevated above normal is diagnostic of neoplasia in the subject.

The present invention further provides a method for assessing the efficacy of therapy to treat neoplasia in a subject who has undergone or is undergoing treatment for neoplasia, by assaying a diagnostic sample of the subject for expression of at least one protocadherin selected from the group consisting of PCDH8, PCDH9, and PCDH17, wherein normal expression of PCDH8, PCDH9, and PCDH17 in the diagnostic sample is indicative of successful therapy to treat neoplasia, and wherein PCDH8 expression decreased below normal in the diagnostic sample and/or PCDH9 expression decreased below normal in the diagnostic sample and/or PCDH7 expression elevated above normal in the diagnostic sample is indicative of a need to continue therapy to treat neoplasia.

The present invention also provides a method for assessing the prognosis of a subject who has neoplasia, by assaying a diagnostic sample of the subject for expression of at least one protocadherin selected from the group consisting of PCDH8, PCDH9, and PCDH17, wherein the subject's prognosis improves with an increase in expression of PCDH8 in the diagnostic sample and/or an increase in expression of PCDH9 in the diagnostic sample and/or a decrease in expression of PCDH17 in the diagnostic sample, and wherein the subject's prognosis worsens with a decrease in expression of PCDH8 in the diagnostic sample and/or a decrease in expression of PCDH9 in the diagnostic sample and/or an increase in expression of PCDH17 in the diagnostic sample.

Additionally, the present invention provides a method for determining whether a subject has neoplasia, by assaying a diagnostic sample of the subject for methylation of PCDH8 promoter and/or methylation of PCDH9 promoter, wherein detection of methylation of the PCDH8 promoter and/or the PCDH9 promoter is diagnostic of neoplasia in the subject.

The present invention further provides a method for assessing the efficacy of therapy to treat neoplasia in a subject who has undergone or is undergoing treatment for; neoplasia, by assaying a diagnostic sample of the subject for methylation of PCDH8 promoter and/or methylation of the PCDH9 promoter, wherein no detection of methylation of the PCDH8 promoter and/or the PCDH9 promoter in the diagnostic sample is indicative of successful therapy to treat neoplasia, and wherein detection of methylation of the PCDH8 promoter and/or the PCDH9 promoter in the diagnostic sample is indicative of a need to continue therapy to treat neoplasia.

The present invention also provides a method for assessing the prognosis of a subject who has neoplasia, by assaying a diagnostic sample of the subject for methylation of PCDH8 promoter and/or PCDH9 promoter, wherein the subject's prognosis improves with a decrease in methylation of the PCDH8 promoter and/or the PCDH9 promoter in the diagnostic sample, and wherein the subject's prognosis worsens with an increase in methylation of the PCDH8 promoter and/or the PCDH9 promoter in the diagnostic sample.

Furthermore, the present invention provides a method for determining whether a subject has neoplasia, by assaying a diagnostic sample of the subject for at least one mutation selected from the group consisting of a PCDH8 mutation and a PCDH17 mutation.

Additionally, the present invention provides a kit for use in detecting neoplasia, including: (a) at least one agent reactive with PCDH8, PCDH9, and/or PCDH17; and (b) reagents suitable for detecting expression of at least one protocadherin selected from the group consisting of PCDH8, PCDH9, and PCDH17. Also provided is a kit for use in detecting neoplasia, including: (a) at least one nucleic acid probe that hybridizes to nucleic acid encoding PCDH8, PCDH9, and/or PCDH17; and (b) reagents suitable for detecting expression of at least one nucleic acid encoding at least one protocadherin selected from the group consisting of PCDH8, PCDH9, and PCDH17.

The present invention also provides a method for treating or preventing neoplasia in a subject, by modulating level and/or function of at least one protocadherin in the subject, wherein the at least one protocadherin is selected from the group consisting of PCDH8, PCDH9, and PCDH17.

The present invention further provides a pharmaceutical composition that includes a modulator of PCDH8 or PCDH9 expression, or a PCDH8 or PCDH9 protein, in an amount effective to treat or prevent neoplasia in a subject to whom the composition is administered, and a pharmaceutically-acceptable carrier. Also provided is a pharmaceutical composition that includes an agent reactive with PCDH17, in an amount effective to treat or prevent neoplasia in a subject to whom the composition is administered, and a pharmaceutically-acceptable carrier.

Additionally, the present invention provides a method for identifying an agent for use in treating and/or preventing neoplasia, by: (a) obtaining a collection of cells having at least one characteristic selected from the group consisting of PCDH8 expression decreased below normal, PCDH9 expression decreased below normal, and PCDH17 expression elevated above normal; (b) contacting a candidate agent with the cells; and (c) determining the effect, if any, of the candidate agent on PCDH8 expression, PCDH9 expression, and/or PCDH17 expression in the cells. Also provided are an agent identified by this method, and use of this agent in a method for treating or preventing neoplasia in a subject.

Finally, the present invention provides a method for identifying an agent for use in treating and/or preventing neoplasia, by: (a) obtaining an animal comprising cells having at least one characteristic selected from the group consisting of PCDH8 expression decreased below normal, PCDH9 expression decreased below normal, and PCDH17 expression elevated above normal; (b) administering a candidate agent to the animal; and (c) determining the effect, if any, of the candidate agent on PCDH8 expression, PCDH9 expression, and/or PCDH17 expression in the animal. Also provided are an agent identified by this method and use of this agent in a method for treating or preventing neoplasia in a subject.

Additional aspects of the present invention will be apparent in view of the description which follows.

### BRIEF DESCRIPTION OF THE FIGURES

FIG. 1 confirms the deletion of chromosome 13q in breast cancer cell lines. (A) HCC1395 harbors a homozygous deletion. Genes that are present in the cell line are shown in white; genes containing deletions are shown in red. (B) Deletion of 13q14 in HCC1395 is confirmed by Southern analysis. An X chromosome probe was used to demonstrate equal loading of DNA. left panel: PCDH17 probe is deleted in HCC1395 tumor DNA (T), but not in its corresponding normal DNA (N). right panel: A control probe on the 0× chromosome hybridizes to both normal (N) and tumor (T) DNA. (C) PCDH9 is homozygously deleted in MDA-MB-436.

FIG. 2 sets forth a summary of deletion data. A map of chromosome 13 is presented, showing selected markers and genes, homozygous deletions in breast cancer cell lines HCC1395 and MDA-MB-436, and relationship of the deletions to LOH and linkage data. Protocadherins are shown in red. Br Ca=breast cancer, LOH=loss of heterozygosity; Pr Ca=prostate cancer.

FIG. 3 illustrates protocadherin expression in cultured breast tissue. Depicted is an RT-PCR analysis of the expression of PCDHs 8, 9, 20, and 17 in cultured and immortalized breast epithelial cells. MCF7 was used as a positive control for PCR. *=immortalized by E6 or E6/E7

FIG. 4 depicts protocadherin expression in mouse tissue. (A) in situ hybridization showing expression of PCDHs 8, 9, 17, and 20 in mouse hippocanpus. (B) Hematoxylin and eosin (Ha&) preparation of breast duct showing luminal and basal/ myoopithelial cells.

FIG. 5 illustrates protocadherin expression in breast cancer. (A) PCDH8 is downregulated in breast cancer cell lines and xenografts. Control RT-PCR of Diaphanous 3 is affected only in HCC1395—the cell line in which it is deleted. (B) PCDH9 is downregulated in breast-cancer xenografts Bx 31, Bx 32, and Bx 36. (C) PCDH17 is upregulated in breast-cancer xenografts, while PCDH20 is downregulated in breast cancer xenograft Bx 36.

FIG. 6 shows patterns of expression of PCDHs 8, 9, 17, and 20 in normal and tumor breast epithelial cells. (A) Patterns of altered PCDH expression in tumors. (B) Cell lines lacking PCDH17 expression.

FIG. 7 depicts methylation of a PCDH8 promoter. (A) A CpG island of PCDH8 is methylated in cancer cell lines. (B) A CpG island of PCDH8 is methylated in primary breast tumors.

FIG. 8 shows that expression of PCDH8 can be restored by 5-aza-deoxycytidine.

FIG. 9 illustrates mutation analyses of PCDH8 in breast cancer, and shows: that PCDH8 is mutated in breast cancer. (A) Somatic mutations. (B) Germline mutations.

FIG. 10 illustrates mutation analyses of PCDH17 in breast cancer, and shows that PCDH17 is mutated in breast cancer cell line MDA-MB-453. Wt=wild-type

FIG. 11 presents a model showing the possible results of deregulation of protocadherins, including loss of cell-cell adhesion/polarity, epithelial-mesenchymal transition, proliferation, and invasion/metastasis.

FIG. 12 shows expression of PCDH8 in normal breast and breast tumors. (A) RT-PCR and Western blot analysis reveals PCDH8 expression in a control cell line (MCF7) and the immortalized lines M2, M3, and MCF10A (10A), and loss of expression of PCDH8 in HCC1395 (1395). (B) PCDH8 mRNA is expressed in murine hippocampus and breast duct by in situ hybridization. H&E of breast duct (1000×). (C) PCDH8 expression is lost in multiple breast cancer cell lines, HCC1395, ZR75-30 (75-30), MDA-MB-435s (435s), and MDA-MB-436 (436), by RT-PCR and Western blot.

FIG. 13 illustrates downregulation of PCDH8 in breast tumors. (A) LOH at markers D13S1305, D13S155, and D13S1228 is found in tumor 68T. Heterozygosity is seen in the corresponding normal tissue, 68N. A 50% or greater reduction in peak intensity was scored as a loss. The position of markers relative to the PCDH8 locus is mapped. (B) Inactivation of PCDH8 by somatic mutation in two tumors: one missense mutation G436A:E146K is found in the extracellular domain of PCDH8 in tumor 68T, and another missense mutation, G1028A:R343H, in breast cancer cell line HCC1599. (C) Southern analysis of methylation of the PCDH8 promoter. DNA was digested with one or more restriction enzymes and electrophoresed in lanes 1-4, where lane 1 corresponds to digestion with RsaI, lane 2 RsaI and CfoI, lane 3 RsaI and HpaII, and lane 4 RsaI and MspI. CfoI and HpaII are methylation sensitive enzymes; MspI is the methylation insensitive isoschizomer of HpaII. Methylation is detected in the breast cancer cell lines ZR-75-30 (75-30) and MDA-MB-435s (MDA-435s), and breast tumors 21T, 95T, and 584T. Normal breast samples and tumor 33T lack methylation of PCDH8. An ANKRD3 control blot shows completion of digestion and serves as a loading control. (D) Restriction map of PCDH8 promoter. RsaI sites are denoted by tall lines labelled “R”. CfoI, HpaII, and MspI sites containing CpG's are denoted by short lines. (E) PCDH8 is reactivated in MDA-MB-435s treated with 5-aza-deoxycytidine (5AdC) but not PBS control. (F) Loss of expression of PCDH8 in tumors 21T and 95T correlates with promoter methylation, as shown in C. Adjacent normal breast lobules and ducts exhibit membranous and cytoplasmic staining of PCDH8 in breast epithelial cells (400×). (G) PCDH8 is downregulated in breast cancer cells relative to adjacent normal breast duct cells in a breast tumor biopsy (100×). Arrowhead=normal cells. Arrow=tumor cells.

FIG. 14 illustrates that PCDH8 expression alters adhesion, migration, and proliferation in normal breast epithelial cells. (A) Expression of PCDH8 (10A-PCDH8), PCDH8K (10A-PCDH8K), and PCDH8H (10A-PCDH8H) in MCF10A. (B) Subcellular localization of PCDH8 and PCDH8K were determined by immunofluorescence using anti-MYC 9E10 antibodies. While PCDH8 localizes at cell processes and cell-cell junctions, PCDH8K localizes to the cytoplasm and is concentrated in perinuclear regions. Corresponding DAPI stain of PCDH8 and PCDH8K transfected cells (400×). (C) Wild type PCDH8 expression in MCF10A increases plating efficiency relative to control cells. Expression of two mutants of PCDH8, PCDH8K and PCDH8H, show attenuated levels of adhesion. (D)-(E) PCDH8 expression inhibits migration. (D) Migration through transwell filters in the presence of a chemoattractant gradient is inhibited by PCDH8. (E) Wound healing assay reveals reduced migration of 10A-PCDH8 relative to control cells (100×). (F) PCDH8K expression increases proliferation independent of EGF supplementation. (G) PCDH8 expression enhances proliferation in EGF- and serum-rich growth media.

FIG. 15 illustrates that PCDH8K transforms normal breast epithelial cells and suppresses RasV12-mediated migration. (A) PCDH8K can transform MCF10A in 2-dimensional culture (plastic) and 3-dimensional culture (left Matrigel panel, 40×) conditions. PCDH8K can accelerate acinus development relative to control cells, 10A-PCDH8, and 10A-PCDH8H (right Matrigel panel, 400×). (B)-(C) Quantification of aberrant 10A-PCDH8K foci. (B) When cultured on plastic, foci were counted in 75 cm² flasks. (C) In reduced growth factor Matrigel, multi-branched, transformed structures were counted per 5000 suspended cells. (D)-(E) PCDH8 partially suppresses the MCF10A-RasV12 phenotype. (D) Western blot showing expression of PCDH8 and RasV12 in MCF10A derivatives. (E) Brightfield microscopy image demonstrates that PCDH8 expression in a RasV12 background suppresses migration of cells (10A-P8/RasV12, 40×). E-cadherin expression is present in 10A-BABE vector control but is reduced in 10A-RasV12 cells irrespective of PCDH8 (400×).

FIG. 16 shows homozygous deletion of 13q14-22 in HCC1395. (A) A homozygous deletion of 13q14-21 in HCC1395 was found by RDA analysis and corroborated by SNP chip and PCR analysis. Genes that are deleted include CHM1, PCDH8, GW112, PCDH17, DIAPH3, and TDRD3. A short stretch of DNA between PCDH8 and GW112 was retained. Other homozygous deletions in this vicinity do not appear to affect PCDH8 or any other gene expressed in breast tissue (Cox, C. et al. A survey of homozygous deletions in human cancer genomes. Proc Natl Acad Sci USA 102, 4542-7 (2005)). (B) Southern analysis confirming deletion of 13q21 in HCC1395 tumor (T) DNA but not corresponding normal (N) DNA. The blot was stripped and hybridized with a chromosome X probe to demonstrate equal loading. Arrows denote deleted DNA.

FIG. 17 illustrates the sequence of PCDH8 isoform expressed in breast. The PCDH8 isoform expressed in breast is a protein of 1070 amino acid residues (SEQ ID NO:82). Underlined are the predicted signal peptide sequence and transmembrane domain. A region of greater than 40% homology shared among human protocadherin paralogs 1, 7, 9, 17, 18, 19 is shown in italics. Antibodies were generated against residues 1052-1070, highlighted in bold. The Genbank accession number is AAC70009.

FIG. 18 shows PCDH8 expression improves cell adhesion and alters proliferation. (A)-(B) Retro-viral expression of PCDH8 promotes cell adhesion to a plastic substrate in MDA-MB-468 (A) and ZR75-30 (B). (C)-(D) Proliferation is context dependent. (C) In M2 cells grown in low serum conditions, PCDH8K stimulates proliferation. (D) In ZR75-30 grown in full media, PCDH8 stimulates proliferation.

FIG. 19 shows a summary of somatic mutations of PCDH8 in breast tumors. 116 breast tumors and 21 breast tumor cell lines were sequenced genomically for PCDH8 mutations. Somatic mutations are diagrammed in a cartoon of PCDH8. In addition, several nonsynonymous germline changes were found: G748A:V250M (EC3), A1099G:T367A (EC4), T2015A:L672Q (EC6), and C2625A:H875Q (cytoplasmic tail), as well as a change in the Kozak sequence (-1C to T). HD=homozygous deletion. LOH=loss of heterozygosity. EC=extracellular cadherin repeat. Gray rectangle=signal peptide or transmembrane domain. Black rectangle=cytoplasmic domain.

### DETAILED DESCRIPTION OF THE INVENTION

As disclosed herein, the inventors have discovered that human protocadherins on chromosome 13q14-21 are activated or inactivated, as the case may be, in an oncogenic switch. For example, the inventors have discovered that protocadherin 17 is turned on, and protocadherins 8 and 9 are shut off, in cancer. In normal breast cells, protocadherin 17 is typically silent, while protocadherins 8 and 9 are typically expressed. Contrastingly, in breast cancer, protocadherin 17 is expressed, while protocadherins 8 and 9 are either mutated or silenced through methylation of their promoters. Altered expression of these genes is also seen in prostate cancer.

Accordingly, the present invention provides a method for determining whether a subject has neoplasia. As used herein, the “subject” is a mammal, including, without limitation, a cow, dog, human, monkey, mouse, pig, or rat. Preferably, the subject is a human. The inventors demonstrate herein that patterns of protocadherin expression are: altered in cancer, including breast cancer and prostate cancer. Thus, the method of the present invention comprises assaying a diagnostic sample of the subject for expression of a protocadherin, wherein detection of protocadherin expression elevated above normal, or decreased below normal, as the case may be, is diagnostic of neoplasia in the subject. By way of example, the method of the present invention may comprise assaying a diagnostic sample of the subject for expression of PCDH8, PCDH9, and/or PCDH17, wherein detection of PCDH8 expression decreased below normal is diagnostic of neoplasia in the subject, detection of PCDH9 expression decreased below normal is diagnostic of neoplasia in the subject, and/or detection of PCDH17 expression elevated above normal is diagnostic of neoplasia in the subject.

As used herein, “PCDH” includes both a PCDH (protocadherin) protein (e.g. PCDH8, PCDH9, PCDH17, PCDH20, etc.) and a PCDH analogue. For example, “PCDH8” includes both a PCDH8 (protocadherin 8) protein and a PCDH8, analogue; “PCDH9” includes both a PCDH9 (protocadherin 9) protein and a PCDH9 analogue; and “PCDH17” includes both a PCDH17 (protocadherin 17) protein and a PCDH17 analogue. Unless otherwise indicated, a “protein” shall include a protein, protein domain, polypeptide, or peptide. A “PCDH analogue”, as used herein, is a functional variant of the PCDH protein of interest (e.g. PCDH8, PCDH9, PCDH17, PCDH20, etc.), having PCDH biological activity, which has 60% or greater (preferably, 70% or greater) amino-acid-sequence homology with the PCDH protein of interest. A PCDH “analogue” includes a variant of the PCDH protein that has a homologous three-dimensional conformation. As further used herein, the term “PCDH biological activity” refers to the function/activity of a protein or peptide that demonstrates an ability to influence the generation, termination, and/or course of progression of a neoplasia, as described herein.

As further used herein, the PCDH8, PCDH9, and PCDH17 amino acid sequences are well known in the art. See, e.g. GenBank Accession Nos. AAH36025: NP_{—}002581, 095206, NP_{—}116567, AAK21986, and AAC70009, including conservative substitutions thereof, for PCDH8; see, e.g. GenBank Accession Nos. NP_{—}982354, NP_{—}065136, Q9HC56, and AAF89689, including conservative substitutions thereof, for PCDH9, and see, e.g. GenBank Accession Nos. NP_{—}055274 and 014917, including conservative substitutions thereof, for PCDH17. “Conservative substitutions” are those amino acid substitutions which are functionally equivalent to the substituted amino acid residue, either because they have similar polarity or steric arrangement, or because they belong to the same class as the substituted residue (e.g. hydrophobic, acidic, or basic). The term “conservative substitutions”, as defined herein, includes substitutions having an inconsequential effect on the ability of the PCDH of interest to influence the generation, termination, and/or course of progression of a neoplasia.

PCDH proteins of interest (e.g. PCDH8, PCDH9, PCDH17, PCDH20, etc.) and PCDH analogues may be produced synthetically or recombinantly, or may be isolated from native cells. PCDHs of interest are preferably produced recombinantly, using conventional techniques and cDNA encoding the PCDH proteins (e.g. PCDH8, PCDH9, PCDH17, PCDH20, etc.).

The method of the present invention may be used to determine whether a subject has a neoplasia, thereby permitting the diagnosis of the neoplasia in the subject. As used herein, “neoplasia” refers to the uncontrolled and progressive multiplication of cells of a neoplasm (i.e., neoplastic cells, such as tumor cells), under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of neoplastic cells having the unique trait of loss of normal controls, resulting in unregulated growth, lack of differentiation, local tissue invasion, and/or metastasis.

As used herein, neoplasms include, without limitation, morphological irregularities of cells in tissue of a subject, as well as pathologic proliferation of cells in tissue of a subject, as compared with normal proliferation in the same type of tissue. Additionally, neoplasms include benign tumors and malignant tumors (e.g. breast tumors) that are either invasive or noninvasive. Malignant neoplasms are distinguished from benign in that the former show a greater degree of anaplasia, or loss of differentiation and orientation of cells, and have the properties of invasion and metastasis. Examples of neoplasms or neoplasias which may be assessed, detected, diagnosed, monitored, or treated in accordance with inventions described herein include, without limitation, carcinomas, particularly those of the bladder, breast, cervix, colon, head, kidney, lung, neck, ovary, prostate, and stomach; lymphocytic leukcmias, particularly acute lymphoblastic leukemia and chronic lymphocytic leukemia; myeloid leukemias, particularly acute monocytic leukemia, acute promyelocytic leukemia, and chronic myelocytic leukemia, malignant lymphomas, particularly Burkitt's lymphoma and Non-Hodgkin's lymphoma; malignant melanomas; myeloproliferative diseases; sarcomas, particularly Ewing's sarcoma, hemangiosarcoma, Kaposi's sarcoma, liposarcoma, peripheral neuroepithelioma, and synovial sarcoma; and mixed types of neoplasias, particularly carcinosarcoma and Hodgkin's disease (Beers and Berkow (eds.), The Merck Manual of Diagnosis and Therapy, 17th ed. (Whitehouse Station, N.J.: Merck Research Laboratories, 1999) 973-74, 976, 986, 988, 991). In a preferred embodiment of the present invention, the methods and compositions of the present invention are directed to the assessment, detection, diagnosis, monitoring, and treatment of breast cancer or prostate cancer.

According to the method of the present invention, the diagnostic sample of a subject may be assayed in vitro or in vivo. Where the assay is performed in vitro, a diagnostic sample from the subject may be removed using standard procedures. The diagnostic sample may be tissue, including any bone, brain tissue, breast tissue, colon tissue, muscle tissue, nervous tissue, ovarian tissue, prostate tissue, retinal tissue, skin tissue, or soft tissue, which may be removed by standard biopsy. In addition, the diagnostic sample may be a bodily fluid, including cerebrospinal fluid, pericardial fluid, peritoneal fluid, saliva, serum, sputum, and urine, or a solid, such as feces. Furthermore, the diagnostic sample taken from the subject or patient may be, for example, any tissue known to have a neoplasm, any tissue suspected of having a neoplasm, or any tissue believed not to have a neoplasm.

Protein may be isolated and purified from the diagnostic sample of the present invention using standard methods known in the art, including, without limitation, extraction from a tissue (e.g. with a detergent that solubilizes the protein), where necessary, followed by affinity purification on a column, chromatography (e.g. FTLC and HPLC), immunoprecipitation (e.g. with an antibody to the PCDH of interest), and/or precipitation (e.g. with isopropanol and a reagent such as Trizol). Isolation and purification of the protein may also be followed by electrophoresis (e.g. on an SDS-polyacrylamide gel). Nucleic acid may be isolated from a diagnostic sample using standard techniques known to one of skill in the art.

In accordance with the method of the present invention, neoplasia in a subject may be diagnosed by assaying a diagnostic sample of the subject for expression of a protocadherin, wherein detection of protocadherin expression elevated above normal, or decreased below normal, as the case may be, is diagnostic of neoplasia in the subject. For example, neoplasia may be diagnosed in a subject by assaying a diagnostic sample of the subject for expression of PCDH8, PCDH9, and/or PCDH17, wherein detection of PCDH8 expression decreased below normal is diagnostic of neoplasia in the subject, detection of PCDH9 expression decreased below normal is diagnostic of neoplasia in the subject, and detection of PCDH17 expression elevated above normal is diagnostic of neoplasia in the subject.

As used herein, “expression” means the transcription of a gene into at least one mRNA transcript, or the translation of at least one mRNA into a protein. For example, “expression of PCDH” means the transcription of the PCDH gene of interest into at least one mRNA transcript, or the translation of at least one mRNA into a PCDH protein, as defined above. Accordingly, a diagnostic sample may be assayed for PCDH expression by assaying for PCDH protein, PCDH cDNA, and/or PCDH mRNA. The appropriate form of PCDH will be apparent based on the particular techniques discussed herein. It is also contemplated that the diagnostic sample may be assayed for expression of any or all forms of the PCDH protein of interest (including precursor, endoproteolytically-processed forms, and other forms resulting from post-translational modification) in order to determine whether a subject or patient has neoplasia.

As used herein, the term “elevated above normal” refers to detection (e.g. of expression of PCDH17, etc.) at a level that is significantly greater than the level expected for the same type of diagnostic sample taken from a non-diseased subject or patient (i.e., one who does not have neoplasia) of the same gender and of similar age. As further used herein, “significantly greater” means that the difference between the level (e.g. of expression of PCDH17, etc.) that is elevated above normal, and the expected (normal) level (e.g. of expression of PCDH17, etc.), is of statistical significance.

Preferably, PCDH expression (e.g. PCDH17 expression) elevated above normal is expression of the PCDH (e.g. PCDH17 expression) at a level that is at least 10% greater than the level of PCDH expression (e.g. PCDH17 expression) otherwise expected. Where PCDH expression (e.g. PCDH17 expression) is expected to be absent from a particular diagnostic sample taken from a particular subject or patient, the normal level of PCDH expression for that subject or patient is nil. Where a particular diagnostic sample taken from a particular subject or patient is expected to have a low level of constitutive PCDH expression, that low level is the normal level of PCDH expression for that subject or patient. As disclosed herein, PCDH17 expression is generally absent from non-neoplastic (normal) cells.

Additionally, as used herein, the term “decreased below normal” refers to detection (e.g. of expression of PCDH8, of expression of PCDH9, etc.) at a level that is significantly lower than the level expected for the same type of diagnostic sample taken from a non-diseased subject or patient (i.e., one who does not have neoplasia) of the same gender and of similar age. As further used herein, “significantly lower” means that the difference between the level (e.g. of expression of PCDH8, of expression of PCDH9, etc.) that is decreased below normal, and the expected (normal) level (e.g. of expression of PCDH8, of expression of PCDH9, etc.), is of statistical significance. Preferably, PCDH expression (e.g. expression of PCDH8, expression of PCDH9, etc.) decreased below normal is expression of the PCDH at a level that is at least 10% less than the level of PCDH expression otherwise expected.

Expected or normal levels of PCDH expression (e.g. PCDH8 expression, PCDH9 expression, PCDH17 expression, etc.) for a particular diagnostic sample taken from a subject or patient may be easily determined by assaying non-diseased subjects of a similar age and of the same gender. For example, diagnostic samples may be obtained from at least normal, healthy men between the ages of 25 and 80, to determine the normal quantity of PCDH expression (e.g. PCDH8 expression, PCDH9 expression, PCDH17 expression, etc.) in males. A similar procedure may be followed to determine the normal quantity of PCDH expression (e.g. PCDH8 expression, PCDH9 expression, PCDH17 expression, etc.) in females. Once the necessary or desired samples have been obtained, the normal quantities of PCDH expression (e.g. PCDH8 expression, PCDH9 expression, PCDH17 expression, etc.) in men and women may be determined using a standard assay for quantification, such as flow cytometry, Western-blot analysis, or an ELISA for measuring protein quantities, as described in the art. For example, an ELISA may be run on each sample in duplicate, and the means and standard deviations of the quantity of the PCDH protein of interest (e.g. PCDH8 protein, PCDH9 protein, PCDH17 protein, etc.) may be determined. If necessary, additional subjects may be recruited before the normal quantities of PCDH expression (e.g. PCDH8 expression, PCDH9 expression, PCDH17 expression, etc.) are quantified.

In accordance with the method of the present invention, a diagnostic sample of a subject may be assayed for PCDH expression (e.g. PCDH8 expression, PCDH9 expression, PCDH17 expression, etc.), and PCDH expression may be detected in a diagnostic sample, using assays and detection methods readily determined from the known art (e.g. immunological techniques, hybridization analysis, fluorescence imaging techniques, radiation detection, etc.), as well as any assays and detection methods disclosed herein (e.g. immunoprecipitation, Western-blot analysis, etc.). For example, a diagnostic sample of a subject may be assayed for PCDH expression (e.g. PCDH8 expression, PCDH9 expression, PCDH17 expression, etc.) using an agent reactive with the PCDH of interest.

As used herein, “reactive” means the agent has affinity for, binds to, or is directed against a target of interest (e.g. PCDH8, PCDH9, PCDH17, etc.). As further used herein, an “agent” shall include a protein, polypeptide, peptide, nucleic acid (including DNA or RNA), antibody, Fab fragment, F(ab′)₂ fragment, molecule, compound, antibiotic, drug, and any combination(s) thereof. A Fab fragment is a univalent antigen-binding fragment of an antibody, which is produced by papain digestion. A F(ab′)₂ fragment is a divalent antigen-binding fragment of an antibody, which is produced by pepsin digestion. Preferably, the agent of the present invention is labeled with a detectable marker or label.

In one embodiment of the present invention, the agent reactive with the PCDH of interest (e.g. PCDH8, PCDH9, PCDH17, etc.) is an antibody. As used herein, the antibody of the present invention may be polyclonal or monoclonal. In addition, the antibody of the present invention may be produced by techniques well known to those skilled in the art. Polyclonal antibody, for example, may be produced by immunizing a mouse, rabbit, or rat with purified protein (e.g. PCDH8, PCDH9, PCDH17, etc.). Monoclonal antibody then may be produced by removing the spleen from the immunized mouse, and fusing the spleen cells with myeloma cells to form a hybridoma which, when grown in culture, will produce a monoclonal antibody. It is expected that antibodies that specifically target protocadherin 17, in particular, will lead to tumor regression.

The antibodies used herein may be labeled with a detectable marker or label. Labeling of an antibody, or any other agent, may be accomplished using one of a variety of labeling techniques, including peroxidase, chemiluminescent labels known in the art, and radioactive labels known in the art. The detectable marker or label of the present invention may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker or label may be a radioactive marker, including, for example, a radioisotope. The radioisotope may be any isotope that emits detectable radiation, such as ³⁵S, ³²P, ¹²⁵I, ³H, or ¹⁴C. Radioactivity emitted by the radioisotope can be detected by techniques well known in the art. For example, gamma emission from the radioisotope may be detected using gamma imaging techniques, particularly scintigraphic imaging. Preferably, the agent of the present invention is a high-affinity antibody labeled with a detectable marker or label.

Where the agent of the present invention is an antibody reactive with a PCDH of interest (e.g. PCDH8, PCDH9, PCDH17, etc.), a diagnostic sample taken from the subject may be purified by passage through an affinity column which contains anti-PCDH antibody as a ligand attached to a solid support, such as an insoluble organic polymer in the form of a bead, gel, or plate. The antibody attached to the solid support may be used in the form of a column. Examples of suitable solid supports include, without limitation, agarose, cellulose, dextran, polyacrylamide, polystyrene, sepharose, or other insoluble organic polymers. The anti-PCDH antibody may be further attached to the solid support through a spacer molecule, if desired. Appropriate binding conditions (e.g. temperature, pH, and salt concentration) for ensuring binding of the agent and the antibody may be readily determined by the skilled artisan. In a preferred embodiment, the anti-PCDH antibody is attached to a sepharose column, such as Sepharose 4B.

Where the agent is an antibody, a diagnostic sample of the subject may be assayed for PCDH expression (e.g. PCDH8 expression, PCDH9 expression, PCDH17 expression, etc.) using binding studies that utilize one or more antibodies immunoreactive with a PCDH protein of interest (e.g. PCDH8, PCDH9, PCDH17, etc.), along with standard immunological detection techniques. For example, the PCDH protein eluted from the affinity column may be subjected to an ELISA assay, Western-blot analysis, flow cytometry, or any other immunostaining method employing an antigen-antibody interaction. Preferably, the diagnostic sample is assayed for PCDH expression (e.g. PCDH8 expression, PCDH9 expression, PCDH17 expression, etc.) using Western blotting.

Alternatively, a diagnostic sample of a subject may be assayed for PCDH expression (e.g. PCDH8 expression, PCDH9 expression, PCDH17 expression, etc.) using hybridization analysis of nucleic acid extracted from the diagnostic sample taken from the subject. According to this method of the present invention, the hybridization analysis may be conducted using Northern-blot analysis of mRNA. This method also may be conducted by performing a Southern-blot analysis of DNA using one or more nucleic acid probes, which hybridize to nucleic acid encoding a PCDH of interest (e.g. PCDH8, PCDH9, PCDH17, etc.). The nucleic acid probes may be prepared by a variety of techniques known to those skilled in the art, including, without limitation, the following: restriction enzyme digestion of PCDH nucleic acid; and automated synthesis of oligonucleotides having sequences which correspond to selected portions of the nucleotide sequence of the PCDH nucleic acid of interest, using commercially-available oligonucleotide synthesizers, such as the Applied Biosystems Model 392 DNA/RNA synthesizer.

The nucleic acid probes used in the present invention may be DNA or RNA, and may vary in length from about 8 nucleotides to the entire length of the PCDH-encoding (e.g. PCDH8-encoding, PCDH9-encoding, PCDH17-encoding, etc.) nucleic acid. The nucleic acid used in the probes may be derived from any mammal, including a human. The nucleotide sequences for human PCDH proteins, including PCDH8, PCDH9, and PCDH17, are known in the art. See, e.g. GenBank Accession Nos. BC036025, NM_{—}002590, NM_{—}032949, AY413-493, AY013873 and AF061573, for PCDH8, see, e.g. GenBank Accession Nos. NM_{—}020403, NM_{—}203487, AK057768, and AF169692, for PCDH9; and see, e.g. GenBank Accession Nos. NT_{—}024524 and NM_{—}014459, for PCDH17. Using one of these PCDH sequences as a probe, for example, the skilled artisan could readily clone corresponding PCDH cDNA from other species. In addition, the nucleic acid probes of the present invention may be labeled with one or more detectable markers or labels. Labeling of the nucleic acid probes may be accomplished using one of a number of methods known in the art—e.g. nick translation, end labeling, fill-in end labeling, polynucleotide kinase exchange reaction, random priming, or SP6 polymerase (for riboprobe preparation)—along with one of a variety of labels—e.g. a radioactive label (such as ³⁵S, ³²P, or ³H), a nonradioactive label (such as biotin, fluorescein (FITC), acridine, cholesterol, or carboxy-X-rhodamine (ROX)), or any other detectable marker disclosed herein. Combinations of two or more nucleic acid probes (or primers), corresponding to different or overlapping regions of the PCDH nucleic acid of interest (e.g. nucleic acid encoding PCDH8, PCDH9, PCDH17, etc.), also may be used to assay a diagnostic sample for PCDH expression (e.g. expression of PCDH8, PCDH9, PCDH17, etc.), using, for example, PCR or RT-PCR.

The detection of PCDH expression (e.g. PCDH8 expression, PCDH9 expression, PCDH17 expression, etc.) in the method of the present invention may be followed by an assay to measure or quantify the extent of PCDH expression (e.g. PCDH8 expression, PCDH9 expression, PCDH17 expression, etc.) in a diagnostic sample of a subject. Such assays are well known to one of skill in the art, and may include immunohistochemistry/immunocytochemistry, flow cytometry, mass spectroscopy, Western-blot analysis, or an ELISA for measuring amounts of PCDH protein (e.g. PCDH8, PCDH9, PCDH17, etc.). For example, to use an immunohistochemistry assay, histological (e.g. paraffin-embedded) sections of tissue may be placed on slides, and then incubated with an antibody against the PCDH protein of interest (e.g. PCDH8, PCDH9, PCDH17, etc.). The slides then may be incubated with a second antibody (against the primary antibody), which is tagged to a dye or other colorimetric system (e.g. a fluorochrome, a radioactive agent, or an agent having high electron-scanning capacity), to permit visualization of the PCDH protein of interest (e.g. PCDH8, PCDH9, PCDH17, etc.) that is present in the sections.

It is contemplated that the diagnostic sample in the present invention frequently will be assayed for PCDH expression (e.g. PCDH8 expression, PCDH9 expression, PCDH17 expression, etc.) not by the subject or patient, nor by his/her consulting physician, but by a laboratory technician or other clinician. Accordingly, the method of the present invention further comprises providing to a subjects or patient's consulting physician a report of the results obtained upon assaying a diagnostic sample of the subject or patient for PCDH expression (e.g. PCDH8 expression, PCDH9 expression, PCDH17 expression, etc.).

Additionally, the present invention provides a method for determining whether a subject has neoplasia, by assaying a diagnostic sample of the subject for methylation of a protocadherin promoter (e.g. methylation of a CpG island within a PCDH promoter), wherein detection of methylation of the PCDH promoter is diagnostic of neoplasia in the subject. By way of example, the method for determining whether a subject has neoplasia may comprise assaying a diagnostic sample of the subject for methylation of the PCDH8 promoter and/or methylation of the PCDH9 promoter, wherein detection of methylation of the PCDH8 promoter and/or the PCDH9 promoter is diagnostic of neoplasia in the subject.

In terms of epigenetic changes, it is known that methylation at CpG sites on DNA may affect gene expression. For example, hypomethylation in the promoter regions of proto-oncogenes is seen in many cancers. Additionally, hypermethylation is seen in the promoter regions of tumor-suppressor genes, and is known to give rise to reduced expression. Methylation-sensitive enzymes (Southern blotting/PCR), methylation-specific PCR, immunohistochemical stains, bisulfite sequencing (e.g. combined with cloning), denaturing HPLC, MethyLight assay (real-time quantitative PCR), single nucleotide primer extension (SNuPE), and methylation analysis using restriction-enzyme digestion (combined bisulfite restriction assay, or COBRA), are among the many techniques used to detect methylation changes in DNA.

By way of example, a diagnostic sample of a subject may be assayed for methylation of the PCDH8 promoter and/or methylation of the PCDH9 promoter, in accordance with the method of the present invention, wherein detection of methylation of the PCDH8 promoter and/or the PCDH9 promoter is diagnostic of neoplasia in the subject. The present invention may be used to detect any neoplasia, including any of those described above. In a preferred embodiment, the neoplasia is breast cancer or prostate cancer. In accordance with the method of the present invention, a diagnostic sample may be assayed for methylation of a protocadherin promoter (e.g. the PCDH8 promoter and/or the PCDH9 promoter) in accordance with assays and detection methods readily determined from the known art and/or disclosed herein, including, without limitation, methylation-sensitive enzymes (Southern blotting/PCR), methylation-specific PCR, immunohistochemical stains, bisulfite sequencing (e.g. combined with cloning), denaturing HPLC, MethyLight assay, SNuPE, and COBRA, as described above.

The present invention further provides a method for assessing the efficacy of therapy to treat neoplasia in a subject or patient who has undergone or is undergoing treatment for neoplasia. The method of the present invention comprises assaying a diagnostic sample of the subject or patient for expression of at least one protocadherin (e.g. PCDH8, PCDH9, PCDH17, etc.). By way of example, a diagnostic sample of a subject may be assayed for expression of PCDH8, PCDH9, and/or PCDH17. In accordance with this method of the present invention, normal expression of PCDHg, PCDH9, and PCDH17 in the diagnostic sample is indicative of successful therapy to treat neoplasia. Contrastingly, PCDH8 expression decreased below normal in the diagnostic sample, and/or PCDH9 expression decreased below normal in the diagnostic sample, and/or PCDH7 expression elevated above normal in the diagnostic sample, is indicative of a need to continue therapy to treat neoplasia.

The method of the present invention may be used to assess the efficacy of therapy to treat any neoplasia, including breast cancer, prostate cancer, and all of those described above. The diagnostic sample may be a tissue or a bodily fluid or solid, as described above, and may be assayed for PCDH expression (e.g. PCDH8 expression, PCDH9 expression, PCDH17 expression, etc.) in vitro or in vivo. In addition, the diagnostic sample may be assayed for PCDH expression (e.g. PCDH8 expression, PCDH9 expression, PCDH17 expression, etc.) using all of the various assays and methods of detection and quantification described above. This method of the present invention provides a means for monitoring the effectiveness of therapy to treat neoplasia by permitting the periodic assessment of levels of PCDH expression (e.g. PCDH8 expression, PCDH9 expression, PCDH17 expression, etc.) in a diagnostic sample taken from a subject or patient.

In accordance with the method of the present invention, a diagnostic sample of a subject or patient may be assayed, and levels of PCDH expression (e.g. PCDH8 expression, PCDH9 expression, PCDH17 expression, etc.) may be assessed, at any time following the initiation of therapy to treat neoplasia. For example, levels of PCDH expression (e.g. PCDH8 expression, PCDH9 expression, PCDH17 expression, etc.) may be assessed while the subject or patient is still undergoing treatment for neoplasia.

Where levels of PCDH8 expression and/or PCDH9 expression detected in an assayed diagnostic sample of the subject or patient continue to remain decreased below normal, and/or where levels of PCDH17 expression detected in an assayed diagnostic sample of the subject or patient continue to remain elevated above normal, a physician may choose to continue with the subject's or patient's treatment for the neoplasia. Similarly, where levels of PCDH8 expression and/or PCDH9 expression in an assayed diagnostic sample of the subject or patient do not noticeably increase through successive assessments, and/or levels of PCDH17 expression in an assayed diagnostic sample of the subject or patient do not noticeably decrease through successive assessments, it may be an indication that the treatment for neoplasia is not working, and that treatment doses could be increased or otherwise altered.

On the other hand, where detected levels of PCDH8 expression ad/or PCDH9 expression in an assayed diagnostic sample of the subject or patient increase through successive assessments, and/or where detected levels of PCDH17 expression in an assayed diagnostic sample of the subject or patient decrease through successive assessments, it may be an indication that the treatment for neoplasia is working, and that treatment doses could be decreased or even ceased. Where PCDH8 expression and PCDH9 expression are no longer detected in an assayed diagnostic sample of a subject or patient at levels decreased below normal, and where PCDH17 expression is no longer detected in an assayed diagnostic sample of a subject or patient at levels elevated above normal (e.g. PCDH17 expression is absent from the diagnostic sample), a physician may conclude that the treatment for neoplasia has been successful, and that such treatment may cease.

It is within the confines of the present invention to assess levels of PCDH expression (e.g. PCDH8 expression, PCDH9 expression, PCDH17 expression, etc.) following completion of a subject's or patient's treatment for neoplasia, in order to determine whether the neoplasia has recurred in the subject or patient. Accordingly, an assessment of levels of PCDH expression (e.g. PCDH8 expression, PCDH9 expression, PCDH17 expression, etc.) in an assayed diagnostic sample may provide a convenient way to conduct follow-ups of patients who have been diagnosed with neoplasias. Furthermore, it is within the confines of the present invention to use assessed levels of PCDH expression (e.g. PCDH8 expression, PCDH9 expression, PCDH17 expression, etc.) in an assayed diagnostic sample as a clinical or pathologic staging tool, as a means for determining the extent of neoplasia in the subject or patient, and as a means for ascertaining appropriate treatment options.

The present invention also provides a method for assessing the efficacy of therapy to treat neoplasia in a subject who has undergone or is undergoing treatment for neoplasia, by assaying a diagnostic sample of the subject for methylation of the PCDH8 promoter and/or methylation of the PCDH9 promoter, wherein no detection of methylation of the PCDH8 promoter and/or the PCDH9 promoter in the diagnostic sample is indicative of successful therapy to treat neoplasia, and wherein detection of methylation of the PCDH8 promoter and/or the PCDH9 promoter in the diagnostic sample is indicative of a need to continue therapy to treat neoplasia. The neoplasia may be any of those described above, including breast cancer and prostate cancer. Suitable diagnostic samples, assays, and detection and quantification methods for use in the method of the present invention have already been described.

A correlation exists, in general, between tumor burden and the survival of a patient who has cancer. Therefore, it is also contemplated in the present invention that assaying a diagnostic sample of a subject for PCDH expression (e.g. PCDH8 expression, PCDH9 expression, PCDH17 expression, etc.) may be a useful means of providing information concerning the prognosis of a subject or patient who has neoplasia. Accordingly, the present invention further provides a method for assessing the prognosis of a subject who has neoplasia, by assaying a diagnostic sample of the subject for PCDH expression (e.g. PCDH8 expression, PCDH9 expression, PCDH17 expression, etc.). By way of example, the prognosis of a subject who has neoplasia may be assessed by assaying a diagnostic sample of the subject for expression of PCDH8, PCDH9, and/or PCDH17. In accordance with this method of the present invention, the subject's prognosis improves with an increase in expression of PCDH8 in the diagnostic sample and/or an increase in expression of PCDH9 in the diagnostic sample, the subject's prognosis improves with a decrease in expression of PCDH17 in the diagnostic sample, the subject's prognosis worsens with a decrease in expression of PCDH8 in the diagnostic sample and/or a decrease in expression of PCDH9 in the diagnostic sample, and the subject's prognosis worsens with an increase in expression of PCDH17 in the diagnostic sample. Suitable diagnostic samples, assays, and detection and quantification methods for use in the method of the present invention have already been described. This method of the present invention provides a means for determining the prognosis of a subject or patient diagnosed with neoplasia based upon the level of PCDH expression (e.g. PCDH8 expression, PCDH9 expression, PCDH17 expression, etc.) in an assayed diagnostic sample of the subject or patient.

According to the method of the present invention, a diagnostic sample of a subject or patient may be assayed, and levels of PCDH expression (e.g. PCDH8 expression, PCDH9 expression, PCDH17 expression, etc.) may be assessed, at any time during or following the diagnosis of neoplasia in the subject or patient. For example, levels of PCDH expression (e.g. PCDH8 expression, PCDH9 expression, PCDH17 expression, etc.) in an assayed diagnostic sample may be assessed before the subject or patient undergoes treatment for neoplasia, in order to determine the subject's or patient's initial prognosis. Additionally, levels of PCDH expression (e.g. PCDH8 expression, PCDH9 expression, PCDH17 expression, etc.) in an assayed diagnostic sample may be assessed while the subject or patient is undergoing treatment for neoplasia, in order to determine whether the subject's or patient's prognosis has become more or less favorable through the course of treatment.

By way of example, where levels of PCDH8 expression and/or PCDH9 expression detected in an assayed diagnostic sample of the subject or patient are, or continue to remain, significantly lower than normal, a physician may conclude that the subject's or patient's prognosis is unfavorable. Similarly, where levels of PCDH17 expression detected in an assayed diagnostic sample of the subject or patient are, or continue to remain, significantly higher than normal, a physician may conclude that the subject's or patient's prognosis is unfavorable. Where PCDH8 expression and/or PCDH9 expression in an assayed diagnostic sample of the subject or patient increases through successive assessments, and/or where PCDH17 expression in an assayed diagnostic sample of the subject or patient decreases through successive assessments, it may be an indication that the subject's or patient's prognosis is improving.

Where detected levels of PCDH8 expression and/or PCDH9 expression in an assayed diagnostic sample of the subject or patient do not increase significantly through successive assessments, it may be an indication that the subject's or patient's prognosis is not improving. Similarly, where detected levels of PCDH17 expression in an assayed diagnostic sample of the subject or patient do not decrease significantly through successive assessments, it may be an indication that the subject's or patient's prognosis is not improving. Where PCDH8 expression and/or PCDH9 expression is normal (e.g. detectable, high, etc.) in a diagnostic sample of the subject or patient, a physician may conclude that the subject's or patient's prognosis is favorable. Finally, where PCDH17 expression is normal (e.g. low, absent, etc.) in a diagnostic sample of the subject or patient, a physician may conclude that the subject's or patient's prognosis is favorable.

The present invention also provides a method for assessing the prognosis of a subject who has neoplasia, comprising assaying a diagnostic sample of the subject for methylation of the PCDH8 promoter and/or methylation of the PCDH9 promoter, wherein the subject's prognosis improves with a decrease in methylation of the PCDH8 promoter and/or the PCDH9 promoter in the diagnostic sample, and wherein the subject's prognosis worsens with an increase in methylation of the PCDH8 promoter and/or the PCDH9 promoter in the diagnostic sample. The neoplasia may be any of those described above, including breast cancer and prostate cancer.

The present invention further provides a method for determining whether a subject has neoplasia, by assaying a diagnostic sample of the subject for at least one mutation in a PCDH gene or protein (e.g. a PCDH8 gene mutation, a PCDH8 protein mutation, a PCDH17 gene mutation, a PCDH17 protein mutation, etc.). In one embodiment, the PCDH8 mutation and/or the PCDH17 mutation results in a loss of heterozygosity in chromosome 13. Exemplary PCDH8 mutations include, without limitation, a G436A (E146K) mutation (predicted to change aspartate⁴⁶ to lysine), a G1028A (R343H) mutation (predicted to change arginine³⁴³ to histidine), a G748A (V250M) mutation (predicted to change valine²⁵⁰ to alanine), a C-IT mutation, a A1099G (T367A) mutation (predicted to change threonine³⁶⁷ to alanine) mutation, and a T2015A (L672Q) mutation. See also Table 4. Exemplary PCDH117 mutations include, without limitation, a deletion in exon 1, a C1364T (P222S) mutation, and a G2899T (E733D) mutation. See also Table 5.

The discovery that PCDHs exhibit differential expression in cells displaying neoplasias provides a means of identifying patients with neoplasias, and presents the potential for commercial application in the form of a test for the diagnosis of neoplasias. The development of such a test provides general screening procedures. Such procedures can assist in the early detection and diagnosis of cancers, and can provide a method for the follow-up of patients in whom there has been detection of PCDH8 expression and/or PCDH9 expression decreased below normal, and/or in whom there has been detection of PCDH17 expression elevated above normal.

Accordingly, the present invention further provides a kit for use as an assay of neoplasia, comprising: (a) at least one agent reactive with PCDH8, PCDH9, and/or PCDH17; and (b) reagents suitable for detecting expression of at least one protocadherin selected from the group consisting of PCDH8, PCDH9, and PCDH17. The agents may be any of those described above; in a preferred embodiment, the agent reactive with PCDH8, PCDH9, and/or PCDH17 is an anti-PCDH8, -PCDH9, and/or -PCDH17 antibody. The agents may be used in any of the above-described assays or methods for detecting or quantifying PCDH expression. Preferably, at least one agent of the present invention is labeled with a detectable marker or label.

Similarly, the present invention provides a kit for use as an assay to detect neoplasia, comprising: (a) at least one nucleic acid probe that hybridizes to nucleic acid encoding PCDH8, PCDH9, and/or PCDH17; and (b) reagents suitable for detecting expression of at least one nucleic acid encoding at least one protocadherin (e.g. PCDH8, PCDH9, PCDH17, etc.). Preferably, at least one nucleic acid of the present invention is labeled with a detectable marker or label.

Significant research efforts and resources have been directed toward the elucidation of anti-neoplastic measures, including chemotherapeutic agents, which are effective in treating patients suffering from neoplasia. Effective anti-neoplastic agents include those which inhibit or control the rapid proliferation of cells associated with neoplasms, those which effect regression or remission of neoplasms, and those which generally prolong the survival of patients suffering from neoplasia. Successful treatment of malignant neoplasia, or cancer, requires elimination of all malignant cells, whether they are found at the primary site, or have extended to local/regional areas, or have metastasized to other regions of the body.

Based upon data presented herein, it is believed that protocadherins play a role in the formation of neoplasms. Thus, it is expected that modulation of the levels of PCDHs in cells provides a means for treating and/or preventing cancer and other neoplasias. Accordingly, the present invention further provides a method for treating or preventing neoplasia in a subject, comprising modulating the level and/or function of at least one protocadherin (e.g. PCDH8, PCDH9, PCDH17, etc.) in the subject. Preferably, neoplasia is treated or prevented in a subject by modulating the level and/or function of PCDH8, PCDH9, and/or PCDH17 in the subject. For example, neoplasia may be treated or prevented in a subject by increasing the level and/or function of PCDH8 and/or PCDH9 in the subject, and/or by decreasing the level and/or function of PCDH 17 in the subject. The neoplasia may be any of those described above, but is preferably breast cancer or prostate cancer.

In general, the level and/or function of a PCDH in a subject may be modulated by administering to the subject a modulator of expression of the PCDH. As used herein, a “modulator of expression” may be any agent or combination of agents that has an antagonistic (inhibitory) or agonistic (facilitatory) effect on expression of a specified protein. Thus, a modulator of PCDH expression may be a PCDH agonist or a PCDH antagonist. The modulators of the present invention include any protein, polypeptide, peptide, nucleic acid (including DNA or RNA), antibody, Fab fragment, F(ab′)₂ fragment, molecule, compound, antibiotic, or drug. Furthermore, the modulators of the present invention include any agent reactive with a PCDH of interest (e.g. PCDH8, PCDH9, etc.) that induces or upregulates expression of that PCDH, and any agent reactive with a PCDH of interest (e.g. PCDH17, etc.) that downregulates expression of that PCDH.

Modulators of PCDHs may be identified using a simple screening assay, including screening procedures well known in the art or disclosed herein. For example, to screen for candidate modulators of PCDHs, cells from a breast tumor line (e.g. MDA-MB-330, MDA-MB-436, MDA-MB-453, HCC-1395, DU4475, Hs578T, ZR75-30, UACC812, etc.) may be plated onto microtiter plates, then contacted with a library of drugs. Any resulting increase in (or upregulation of) PCDH expression, and/or any resulting decrease in (or downregulation of PCDH expression, then may be detected using nucleic acid hybridization and/or immunological techniques known in the art, including an ELISA. Modulators of PCDHs will include those agents/drugs that induce or upregulate expression of a PCDH, and those agents/drugs which decrease or downregulate expression of a PCDH. In this manner, candidate modulators also may be screened for their ability to inhibit proliferation of neoplasms, using PCDH expression as an indicator that cell division or growth of cells in a neoplasm is decreasing in rate, or has stopped.

It is within the confines of the present invention that the modulator of PCDH expression may be linked to another agent, or administered in combination with another agent (such as an anti-neoplastic drug or a ribozyme), in order to increase the effectiveness of the treatment of neoplasia and/or increase the efficacy of targeting. Examples of anti-neoplastic drug to which the modulator of PCDH expression may be linked include, without limitation, carboplatin, cyclophosphamide, doxorubicin, etoposide, and vincristine.

In accordance with the method of the present invention, neoplasia also may be treated or prevented in a subject by increasing the level and/or function of a PCDH (e.g. PCDH8, PCDH9, etc.) in the subject. Preferably, the level and/or function of a PCDH of interest (e.g. PCDH8, PCDH9, etc.) in the subject is increased by at least 10% in the method of the present invention. More preferably, the level and/or function of a PCDH of interest (e.g. PCDH8, PCDH9, etc.) is increased by at least 20%.

By way of example, the level and/or function of a PCDH (e.g. PCDH8, PCDH9, etc.) in a subject may be increased by directly or indirectly increasing levels of the PCDH in vivo within the subject. For example, the level of a PCDH (e.g. PCDH8, PCDH9, etc.) in a subject may be increased by administering the PCDH protein to the subject, in an amount effective to treat neoplasia in the subject. Similarly, the level of a PCDH (e.g. PCDH8, PCDH9, etc.) in a subject may be increased by administering to the subject a nucleic acid sequence encoding the PCDH, in a manner permitting expression of the PCDH protein in the subject, and in an amount effective to treat the neoplasia.

Additionally, the level and/or function of a PCDH of interest (e.g. PCDH8, PCDH9, etc.) in a subject also may be increased by directly or indirectly causing, inducing, or stimulating the upregulation of expression of the PCDH within a subject. In the method of the present invention, a PCDH (e.g. PCDH8, PCDH9, etc.) in a subject may be unregulated, for example, by administering to the subject a modulator of PCDH expression, such as a small molecule or protein mimetic. Accordingly, in one embodiment of the present invention, the level and/or function of a PCDH of interest (e.g. PCDH8, PCDH9, etc.) is increased in a subject by administering to the subject a modulator of PCDH expression (e.g. a modulator of PCDH8 expression, a modulator of PCDH9 expression, etc.), in an amount effective to treat the neoplasia in the subject.

In accordance with the method of the present invention, the level and/or function of a PCDH of interest (e.g. PCDH8, PCDH9, etc.) in a subject also may be increased by targeting the PCDH directly or indirectly. The level and/or function of the PCDH may be increased indirectly by targeting an enzyme or other endogenous molecule that regulates or modulates the function of the PCDH in the subject. By way of example, the level and/or function of a PCDH of interest (e.g. PCDH8, PCDH9, etc.) in a subject may be increased by directly or indirectly activating, facilitating, or stimulating one or more functions of the PCDH in the subject (e.g. by the modulation or regulation of enzymes, other proteins, lipids, etc., that interact with the PCDH). The term “activating”, as used herein, means stimulating or inducing one or more functions of the PCDH in the subject, particularly those functions associated with the suppression or inhibition of neoplasias, as disclosed herein. In the method of the present invention, a PCDH (e.g. PCDH8, PCDH9, etc.) in a subject may be activated, for example, by administering to the subject a small molecule or protein mimetic that stimulates the PCDH (e.g. PCDH8, PCDH9, etc.) or that is reactive with the PCDH (e.g. PCDH8, PCDH9, etc.), in an amount effective to treat the neoplasia in the subject.

Activators of PCDH function may be identified using a functional screening assay, including any well known in the art and/or disclosed herein. For example, to screen for candidate activators of PCDH function, cells known to exhibit at least one PCDH function (e.g. function of PCDH8, PCDH9, etc.) may be plated onto microtiter plates, then contacted with a library of candidate agents/drugs. Any resulting increase in PCDH function (e.g. synaptic function, contact-inhibition of cell proliferation, suppression of tumor formation, control of gene expression, etc.) then may be detected using techniques known in the art. Activators of PCDH function will include those agents/drugs which increase at least one function of a PCDH. The function of a PCDH of interest may be measured using standard techniques known to one of skill in the art.

Once the candidate agent of the present invention has been screened, and has been determined to have a suitable activating effect on the PCDH of interest (e.g. PCDH8, PCDH9, etc.), it may be evaluated for its effect on tumor-cell proliferation. In particular, the candidate agent may be assessed for its ability to act as an inhibitor to cell division or to otherwise function as an appropriate tumor-suppressing agent. It is expected that the PCDH activator of the present invention will be useful to treat neoplasms, including those disclosed herein. Preferably, the PCDH activator of the present invention is a PCDH8 activator and/or a PCDH9 activator.

A PCDH protein (e.g. PCDH8, PCDH9, etc.), a PCDH nucleic acid, a modulator of PCDH expression, and an activator of PCDH function may be administered to a subject who has neoplasia, either alone or in combination with one or more anti-neoplastic drugs used to treat neoplasias. Examples of anti-neoplastic drugs with which the PCDH protein may be combined have already been described.

Furthermore, the level and/or function of a PCD1:I of interest (e.g. PCDH8, PCDH9, etc.) may be increased in a subject by altering chromatin silencing or PCDH methylation in the subject, or by otherwise reversing gene silencing of the PCDH. Chromatin is the natural substrate for the control of gene expression; it contains DNA, transcriptional machinery, and structural proteins (such as histories). It has been shown that transcriptional activity of a gene is largely controlled by packaging of the template within chromatin (Jones and Wolffe, Relationships between chromatin organization and DNA methylation in determining gene expression. Semin. Cancer Biol., 9(5):339-47, 1999). Histone acetylation and DNA methylation alter the nucleosomal infrastructure, thereby repressing or activating transcription. These covalent modifications have causal roles in promoter-specific events, and in the global control of chromosomal activity. Histone acetylation and DNA methylation also have a major impact in both oncogenic transformation and normal development. See, e.g. Jones and Wolffe, Relationships between chromatic organization and DNA methylation in determining gene expression. Semin. Cancer Biol., 9(5):339-47, 1999.

It is also known that two epigenetic processes—densely-methylated DNA in association with transcriptionally-repressive chromatin characterized by the presence of underacetylated histones—are dynamically linked (Cameron et al., Synergy of demethylation and histone deacetylase inhibition in the re-expression of genes silenced in cancer. Nat. Genet., 21:103-07, 1999). MeCP2, a methyl-CpG-binding protein, appears to reside in a complex with histone deacetylase activity. MeCP2 can mediate formation of transcriptionally-repressive chromatin on methylated promoter templates in vitro, and this process can be reversed by trichostatin A (TSA), a specific inhibitor of histone deacetylase. It has also been shown that, following minimal demethylation and slight gene reactivation in the presence of low-dose 5-aza-2′deoxycytidine (5Aza-dC), TSA treatment results in robust re-expression and transcriptional reactivation of hypermethylated genes MLH1, TIMP3 (TIMP3), CDKN2B (INK4B, p15), and CDKN2A (INK4, p16). Accordingly, in one embodiment of the present invention, activity of PCDH8 or PCDH9 is increased in the subject by administering 5-aza-deoxycytidine (e.g. 5-aza-2′deoxycytidine) and/or trichostatin A to the subject.

In accordance with the method of the present invention, neoplasia also may be treated or prevented in a subject by decreasing the level and/or function of a PCDH (e.g. PCDH17, etc.) in the subject. Preferably, the level and/or function of a PCDH of interest (e.g. PCDH17, etc.) in the subject is decreased by at least 10% in the method of the present invention. More preferably, the level and/or function of a PCDH of interest (e.g. PCDH17, etc.) is decreased by at least 20%.

By way of example, the level and/or function of a PCDH (e.g. PCDH17, etc.) in a subject may be decreased by directly or indirectly decreasing levels of the PCDH in vivo within the subject. A decrease in the level of the PCDH in the subject may be achieved, for example, by directly or indirectly causing, inducing, or stimulating the downregulation of expression of the PCDH within a subject. Accordingly, in one embodiment of the present invention, the level and/or function of a PCDH of interest (e.g. PCDH17, etc) is decreased in a subject by administering to the subject a modulator of PCDH expression (e.g. a modulator of PCDH17 expression, etc.), such as a small molecule or protein mimetic, in an amount effective to treat the neoplasia in the subject.

In accordance with the method of the present invention, the level and/or function of a PCDH of interest (e.g. PCDH17, etc.) in a subject also may be decreased by targeting the PCDH directly or indirectly. The level and/or function of the PCDH may be decreased indirectly by targeting an enzyme or other endogenous molecule that regulates or modulates the function of the PCDH in the subject.

By way of example, the level and/or function of a PCDH of interest (e.g. PCDH17, etc.) in a subject may be decreased by directly or indirectly blocking, deactivating, inhibiting, or preventing one or more functions of the PCDH in the subject (e.g. by the modulation or regulation of proteins that interact with the PCDH), or by diminishing the amount of the PCDH in the subject. The term “inhibiting”, as used herein, means disabling, disrupting, or inactivating one or more functions of the PCDH in the subject, particularly those functions associated with the formation and growth of neoplasias, as disclosed herein.

In the method of the present invention, a PCDH (e.g. PCDH17, etc.) in a subject may be inhibited, for example, by administering to the subject a small molecule or protein mimetic that inhibits the PCDH (e.g. PCDH17, etc.) and/or that is reactive with the PCDH (e.g. PCDH17, etc.), in an amount effective to treat the neoplasia in the subject. By way of example, a PCDH of interest (e.g. PCDH17, etc.) may be inhibited in a subject who has neoplasia by administering a PCDH inhibitor to the subject. As used herein, “a PCDH inhibitor” shall include a protein, polypeptide, peptide, nucleic acid (including DNA, RNA, and an antisense oligonucleotide), antibody (monoclonal and polyclonal, as described above), Fab fragment (as described above), F(ab′)₂ fragment (as described above), molecule, compound, antibiotic, drug, or any combination thereof. PCDH inhibitors (e.g. inhibitors of PCDH17) provide novel and valuable tools for treating and preventing neoplasia. For example, since PCDH17 is generally not expressed in non-diseased subjects, inhibition of PCDH17 should have a therapeutic effect without resulting in the harmful or deleterious side-effects that frequently accompany therapy using other anti-neoplastic drugs or radiation. Exemplary PCDH inhibitors include, without limitation, oligonucleotides antisense to the PCDH of interest (e.g. PCDH17, etc.), RNAi directed against the PCDH of interest (e.g. PCDH17, ctc.), agents reactive with the PCDH of interest (e.g. PCDH17, etc.), a dominant-negative form of the PCDH of interest (e.g. PCDH17, etc.), and a constitutively-active form of the PCDH of interest (e.g. PCDH17, etc.).

It is well understood in the art that a gene may be silenced at a number of stages, including, without limitation, pre-transcription silencing, transcription silencing, translation silencing, post-transcription silencing, and post-translation silencing. Accordingly, in one embodiment of the present invention, the level and/or function of a PCDH of interest (e.g. PCDH17, etc.) is decreased in the subject by gene-silencing the PCDH using antisense technology or interference RNA (RNAi).

RNA interference (RNAi) is an RNA-mediated, sequence-specific gene-silencing mechanism. RNAi, a double-stranded (ds) interference RNA, was discovered by Guo and Kemphues in 1995, when they reported that both the sense and antisense strands of test oligonucleotides disrupted the expression of par-1 in Caenorhabditis elegans, following injection into a cell (Guo et al., Par-1, A gene required for establishing polarity in C. elegans embryos, encodes a putative Ser/Thr kinase that is asymmetrically distributed. Cell, 81:61 1-20, 1995). In 1998, Fire et al. clearly proved the existence and efficacy of RNAi by injecting into the gut of C. elegans a dsRNA that had been prepared in vitro (Fire et al., Potent and specific genetic interference by double-stranded RNA in Caenorhabditis elegans, Nature, 391:806-11, 1998). The injection of dsRNA into C. elegans resulted in loss of expression of the homologous target gene, not only throughout the worm, but also in its progeny. It is now well accepted that the phenomenon of RNAi is ubiquitous among bacteria, fungi, plants, and animals.

As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment, or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

The RNAi of the present invention may be included within a plasmid, although it is to be understood that other types of nucleic acids, such as viral vectors, may also be used for the purposes of the present invention. The term “plasmid”, as used herein, refers generally to circular double-stranded DNA that is not bound to a chromosome. The DNA may be a chromosomal or episomal-derived plasmid. The plasmid of the present invention may optionally contain a terminator of transcription; a promoter, and/or a discrete series of restriction-endonuclease recognition sites, located between the promoter and the terminator. In a preferred embodiment, a polynucleotide insert of interest (e.g. one encoding the RNAi) should be operatively linked to an appropriate promoter, such as its native promoter or a host-derived promoter, such as a tissue-specific promoter or an inducible promoter. Other suitable promoters will be known to the skilled artisan.

In one embodiment of the present invention, RNAi is produced in vivo by an expression vector containing a gene-silencing cassette coding for RNAi or any other a post-transcription gene-silencing composition. See, e.g. U.S. Pat. No. 6,278,039, C. elegans deletion mutants; U.S. Patent Application No. 2002/0006664, Arrayed transfection method and uses related thereto; WO 99/326197 Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes. In another embodiment of the present invention, RNAi is produced in vitro, synthetically or recombinantly, and transferred into the subject using standard molecular-biology techniques. Methods of making and transferring RNAi are well known in the art: see, e.g. Ashrafi et al., Genome-wide RNAi analysis of Caenorhabditis elegans fat regulatory genes. Nature, 421:268-72, 2003; Cottrell et al., Silence of the strands: RNA interference in eukaryotic pathogens. Trends Microbiol., 11:37-43, 2003; Nikolaev et al., Parc: A cytoplasmic anchor for p53. Cell, 112:29-40, 2003, Wilda et al., Killing of leukemic cells with a BCR/ABL fusion gene RNA interference (RNAi), Oncogene, 21:5716-24, 2002; Escobar et al., RNAi-mediated oncogene silencing confers resistance to crown gall tumorigenesis. Proc. Natl. Acad. Sci. USA, 98:13437-42, 2001; and Billy et al., Specific interference with gene expression induced by long, double-stranded RNA in mouse embryonal teratocarcinoma cell lines. Proc. Natl. Acad. Sci. USA, 98:14428-33, 2001.

A PCDH of interest (e.g. PCDH17, etc.) also may be silenced with antisense technology. An antisense oligonucleotide (e.g. DNA, RNA, etc.) is a molecule with a sequence complementary to a specific RNA transcript, or mRNA; upon binding, the antisense oligonucleotide prevents further processing of the transcript or translation of the mRNA. In accordance with this embodiment of the present invention, the PCDH inhibitor is an oligonucleotide antisense to the PCDH of interest (e.g. PCDH17, etc.).

Oligonucleotides antisense to a PCDH of interest (e.g. PCDH17, etc.) may be designed based on the nucleotide sequence of the PCDH. For example, a partial sequence of the PCDH nucleotide sequence (generally, 18-20 base pairs), or a variation sequence thereof, may be selected for the design of an antisense oligonucleotide. This portion of the PCDH nucleotide sequence may be within the 5′ domain. A nucleotide sequence complementary to the selected partial sequence of the PCDH gene, or the selected variation sequence, then may be chemically synthesized using one of a variety of techniques known to those skilled in the art, including, without limitation, automated synthesis of oligonucleotides having sequences which correspond to a partial sequence of the PCDH nucleotide sequence, or a variation sequence thereof, using commercially-available oligonucleotide synthesizers, such as the Applied Biosystems Model 392 DNA/RNA synthesizer.

By way of example, antisense molecules may be generated, synthetically or recombinantly, with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are sensitive to nucleases, and have half-lives of 15-30 min in serum. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein et al., Oligodeoxynucleotides as inhibitors of gene expression: a review. Cancer Res., 48:2659-68, 1998). Antisense molecules designed to bind to the entire mRNA may be made by inserting cDNA into an expression plasmid in the opposite or antisense orientation. Antisense molecules may also function by preventing translation initiation factors from binding near the 5′ cap site of the mRNA, or by interfering with interaction of the mRNA and ribosomes. See, e.g. U.S. Pat. No. 6,448,080, Antisense modulation of WRN expression; U.S. Patent Application No. 2003/0018993, Methods of gene silencing using inverted repeat sequences; U.S. Patent Application No., 2003/0017549, Methods and compositions for expressing polynucleotides specifically in smooth muscle cells in vivo; Tavian et al., Stable expression of antisense urokinase mRNA inhibits the proliferation and invasion of human hepatocellular carcinoma cells. Cancer Gene Ther., 10:112-20, 2003; Maxwell and Rivera, Proline oxidase induces apoptosis in tumor cells and its expression is absent or reduced in renal carcinoma. J: Biol. Chem., 278(11):9784-89, 2003; Ghosh et al., Role of superoxide dismutase in survival of Leishmania within the macrophage. Biochem. J., 369:447-52, 2003; and Zhang et al., An anti-sense construct of full-length ATM cDNA imposes a radiosensitive phenotype on normal cells. Oncogene, 17:811-18, 1998.

Once the desired antisense oligonucleotide has been prepared, its ability to inhibit the PCDH of interest (e.g. PCDH17, etc.) then may be assayed. For example, the oligonucleotide antisense to the PCDH may be contacted with tumor cells derived from a breast tumor cell line, and the levels of PCDH expression (e.g. PCDH17 expression, etc.) in the cells may be determined using standard techniques, such as Western-blot analysis. Alternatively, the antisense oligonucleotide may be delivered to tumor cells derived from a breast tumor cell line using a liposome vehicle, and the levels of PCDH expression (e.g. PCDH17 expression, etc.) in the cells may then be determined using standard techniques, such as Western-blot analysis. Where the level of PCDH expression (e.g. PCDH17 expression, etc.) in tumor cells is reduced in the presence of the designed antisense oligonucleotide, it may be concluded that the oligonucleotide could be a useful PCDH inhibitor.

It is within the confines of the present invention that an oligonucleotide antisense to a PCDH of interest (e.g. PCDH17, etc.), or RNAi directed against the PCDH of interest, may be linked to another agent, such as an anti-neoplastic drug or a ribozyme, in order to increase the effectiveness of the treatment, increase the efficacy of targeting, and/or increase the efficacy of degradation of PCDH RNA. Examples of anti-neoplastic drugs to which the antisense or R:NAi oligonucleotide may be linked include, without limitation, carboplatin, cyclophosphamide, doxorubicin, etoposide, and vincristine. Moreover, oligonucleotide antisense to the PCDH of interest (e.g. PCDH17, etc.) may be prepared using modified bases (e.g. a phosphorothioate) to make the oligonucleotide more stable and better able to withstand degradation.

In another embodiment of the present invention, a PCDH of interest (e.g. PCDH17, etc.) in a subject is inhibited by administering to the subject a small molecule or protein mimetic that is reactive with the PCDH. In accordance with this embodiment, the PCDH inhibitor would be an agent reactive with the PCDH of interest (e.g. PCDH17, etc.). Examples of such reactive agents have already been provided. In one preferred embodiment, the agent is an antibody that binds to the PCDH of interest (e.g. PCDH17, etc.). Methods for making anti-PCDH antibodies have been described above.

Additional PCDH inhibitors may be identified using screening procedures well known in the art. For example, to screen for candidate inhibitors of PCDH function, cells known to exhibit at least one PCDH function (e.g. function of PCDH17, etc.) may be plated onto microtiter plates, then contacted with a library of candidate agents/drugs. Any resulting decrease in PCDH function (e.g. synaptic function, contact-inhibition of cell proliferation, suppression of tumor formation, control of gene expression, etc.) then may be detected using techniques known in the art. Inhibitors of PCDH function will include those agents/drugs which decrease at least one function of a PCDH. The function of a PCDH of interest may be measured using standard techniques known to one of skill in the art.

Once the candidate agent of the present invention has been screened, and has been determined to have a suitable inhibitory effect on the PCDH of interest (e.g. PCDH17, etc.)—i.e., it is reactive with the PCDH, it binds the PCDH, or it otherwise inactivates the function of the PCDH)—it may be evaluated for its effect on tumor-cell proliferation. In particular, the candidate agent may be assessed for its ability to act as an inhibitor to cell division or to otherwise function as an appropriate tumor-suppressing agent. It is expected that the PCDH inhibitor of the present invention will be useful to treat neoplasms, including those disclosed herein. Preferably, the PCDH inhibitor of the present invention is a PCDH17 inhibitor.

It is within the confines of the present invention that the agent reactive with a PCDH of interest (e.g. PCDH17, etc.), or any other PCDH inhibitor, may be co-administered to a subject along with an additional therapeutic agent. By way of example, anti-neoplastic drugs with which the reactive agent or other PCDH inhibitor may be co-administered include, without limitation, carboplatin, cyclophosphamide, doxorubicin, etoposide, and vincristine. The reactive agent or other PCDH inhibitor also may be administered to a subject in combination with a fusion protein, such as a VEGF-Trap.

Vascular endothelial growth factor (VEGF) is a critical promoter of blood-vessel growth during embryonic development and tumorigenesis. The VEGF-Trap is a high-affinity, soluble decoy receptor for VEGF. To date, studies of VEGF antagonists have primarily focused on halting progression in models of minimal-residual cancer. Consistent with this focus, recent clinical trials suggest that blockade of VEGF may impede cancer progression, presumably by preventing neoangiogenesis. However, VEGF is also a key mediator of endothelial-vascular mural-cell interactions, a role that may contribute to the integrity of mature vessels in advanced tumors. The VEGF-Trap can achieve high-affinity blockade of VEGF, and abolish mature, preexisting vasculature, in established xenografts, such eradication of vasculature is followed by marked tumor regression. Thus, the contribution of relatively low levels of VEGF to vessel integrity may be critical to maintenance of even very small tumor masses (Huang et al., Regression of established tumors and metastases by potent vascular endothelial growth factor blockade. Proc. Natl. Acad. Sci. USA, 100(13):7785-90, 2003). See also Saishin et al., VEGF-TRAP(R1R2) suppresses choroidal neovascularization and VEGF-induced breakdown of the blood-retinal barrier. Cell Physiol., 195(2):241-48, 2003. Exemplary VEGF-Traps include, without limitation, VEGF-TRAP(R1R2)—a composite decoy receptor that combines ligand binding elements, taken from the extracellular domains of VEGF receptors 1 and 2, fused to the Fc portion of IgG1—and vascular endothelial growth factor Trap(A40).

The present invention contemplates the use of proteins and protein analogues generated by synthesis of polypeptides in vitro (e.g. by chemical means or by in vitro translation of mRNA). For example, PCDHs (e.g. PCDH8, PCDH9, etc.), PCDH inhibitors (e.g. inhibitors of PCDH17, etc.), and other peptides for use in the methods of the present invention may be synthesized by methods commonly known to one skilled in the art (Modern Techniques of Peptide and Amino Acid Analysis (New York: John Wiley & Sons, 1981; Bodansky, M., Principles of Peptide Synthesis (New York: Springer-Verlag New York, Inc., 1984). Examples of methods that may be employed in the synthesis of the amino acid sequences, and analogues of these sequences, include, but are not limited to, solid-phase peptide synthesis, solution-method peptide synthesis, and synthesis using any of the commercially-available peptide synthesizers. Amino acid sequences for use in the present invention may contain coupling agents and protecting groups, which are used in the synthesis of protein sequences, and which are well known to one of skill in the art.

In the method of the present invention, a PCDH protein, a nucleic acid sequence encoding a PCDH, a modulator of PCDH expression, an activator of PCDH function, an agent reactive with a PCDH, a PCDH inhibitor, or any other therapeutic agent for use in treating neoplasia as disclosed herein (referred to herein as any “anti-neoplastic therapeutic agent”), is administered to a subject who has neoplasia in an amount effective to treat the neoplasia in the subject. As used herein, the phrase “effective to treat the neoplasia” means effective to ameliorate or minimize the clinical impairment or symptoms resulting from the neoplasia. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasia, or by limiting, suspending, terminating, or otherwise controlling the maturation and proliferation of cells in the neoplasm. The amount of the anti-neoplastic therapeutic agent of choice that is effective to treat neoplasia in a subject will vary depending on the particular factors of each case, including the type of neoplasia, the stage of neoplasia, the subject's weight, the severity of the subject's condition, and the method of administration. These amounts can be readily determined by the skilled artisan.

In accordance with the method of the present invention, the anti-neoplastic therapeutic agents disclosed herein (e.g. modulators of PCDH expression, PCDH proteins, nucleic acid sequences encoding PCDHs, agents reactive with PCDHs, PCDH inhibitors, etc.) may be administered to a human or animal subject by known procedures, including, without limitation, oral administration, parenteral administration (e.g. epifascial, intracapsular, intracutaneous, intradermal, intramuscular, intraorbital, intraperitoneal, intraspinal, intrasternal, intravascular, intravenous, parenchymatous, or subcutaneous administration), transdermal administration, and administration by osmotic pump. One preferred method of administration is parenteral administration, by intravenous or subcutaneous injection.

For oral administration, the formulation of the anti-neoplastic therapeutic agent of choice may be presented as capsules, tablets, powders, granules, or as a suspension. The formulation may have conventional additives, such as lactose, mannitol, cornstarch, or potato starch. The formulation also may be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the anti-neoplastic therapeutic agent of choice may be combined with a sterile aqueous solution, which is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above.

For transdermal administration, the anti-neoplastic therapeutic agent of choice may be combined with skin penetration enhancers, such as propylene glycol, polyethylene glycol, isopropanol, ethanol, oleic acid, N-methylpyrrolidone, and the like, which increase the permeability of the skin to the anti-neoplastic therapeutic agent, and permit the anti-neoplastic therapeutic agent to penetrate through the skin and into the bloodstream. The composition of enhancer and anti-neoplastic therapeutic agent also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The anti-neoplastic therapeutic agent may be administered transdermally, at or near the site on the subject where the neoplasm is localized. Alternatively, the anti-neoplastic therapeutic agent may be administered transdermally at a site other than the affected area, in order to achieve systemic administration.

The anti-neoplastic therapeutic agent of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the anti-neoplastic therapeutic agent of choice.

Where the anti-neoplastic therapeutic agent of choice is a protein, the protein may be administered to a subject by introducing to the subject the protein itself, or by introducing to the subject a nucleic acid encoding the protein, in a manner permitting expression of the protein. Accordingly, in one embodiment of the present invention, the level and/or function of at least one PCDH (e.g. PCDH8, PCDH9, PCDH17, etc.) in a subject may be modulated, and neoplasia in the subject may be treated, by administering to the subject an amount of the anti-neoplastic therapeutic protein effective to treat neoplasia in the subject. This amount may be readily determined by the skilled artisan. In a further embodiment of the present invention, the level and/or function of at least one PCDH (e.g. PCDH8, PCDH9, PCDH17, etc.) in a subject may be modulated, and neoplasia in the subject may be treated, by administering to the subject a nucleic acid sequence encoding the anti-neoplastic therapeutic protein, in a manner permitting expression of the protein in the subject. The nucleic acid sequence encoding the anti-neoplastic therapeutic protein is administered to the subject in an amount effective to treat neoplasia in the subject. This amount may be readily determined by the skilled artisan.

Anti-neoplastic therapeutic proteins for use in the present invention may be administered or introduced to a subject by known techniques used for the introduction of proteins and other drugs, including, for example, injection and transfusion. Where a neoplasm is localized to a particular portion of the body of the subject, it may be desirable to introduce the therapeutic protein directly to that area by injection or by some other means (e.g. by introducing the protein into the blood or another body fluid).

Anti-neoplastic therapeutic nucleic acids for use in the present invention may be introduced to a subject using conventional procedures known in the art, including, without limitation, electroporation, DEAL dextran transfection, calcium phosphate transfection, lipofection, monocationic liposome fusion, polycationic liposome fusion, protoplast fusion, creation of an in vivo electrical field, DNA-coated microprojectile bombardment, injection with recombinant replication-defective viruses, homologous recombination, in vivo gene therapy, ex vivo gene therapy, viral vectors, naked DNA transfer, or any combination thereof. Recombinant viral vectors suitable for gene therapy include, but are not limited to, vectors derived from the genomes of such viruses as retrovirus, HSV, adenovirus, adeno-associated virus, Semiliki Forest virus, cytomegalovirus, and vaccinia virus. Additionally, it is within the confines of the present invention that a nucleic acid encoding an anti-neoplastic therapeutic protein may be introduced into suitable cells in vitro, using conventional procedures, to achieve expression of the therapeutic protein in the cells. Cells expressing the therapeutic protein then may be introduced into a subject to treat neoplasia in vivo. In such an ex vivo gene therapy approach, the cells are preferably removed from the subject, subjected to DNA techniques to incorporate nucleic acid encoding the therapeutic protein, and then reintroduced into the subject.

It is also within the confines of the present invention that a formulation containing an anti-neoplastic therapeutic agent for use in treating neoplasia (e.g. a modulator of PCDH expression, a PCDH protein, a nucleic acid sequence encoding a PCDH, an agent reactive with a PCDH, a PCDH inhibitor, etc.) may be further associated with a pharmaceutically-acceptable carrier, thereby comprising a pharmaceutical composition. Accordingly, the present invention further provides a pharmaceutical composition, comprising an anti-neoplastic therapeutic agent for use in treating neoplasia, as disclosed herein (e.g. a modulator of PCDH expression, a PCDH protein, a nucleic acid sequence encoding a PCDH, an agent reactive with a PCDH, a PCDH inhibitor, etc.), and a pharmaceutically-acceptable carrier. The pharmaceutically-acceptable carrier must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arable, lactose, magnesium stearate, methyl cellulose, powders, saline, sodium alginate, sucrose, starch, talc, and water, among others. Formulations of the pharmaceutical composition may be conveniently presented in unit dosage.

The pharmaceutical formulations of the present invention may be prepared by methods well-known in the pharmaceutical arts. For example, the anti-neoplastic therapeutic agent of choice may be brought into association with a carrier or diluent, as a suspension or solution. Optionally, one or more accessory ingredients (e.g. buffers, flavoring agents, surface active agents, and the like) also may be added. The choice of carrier will depend upon the route of administration. The pharmaceutical composition would be useful for administering the anti-neoplastic therapeutic agent of the present invention to a subject to treat neoplasia. The anti-neoplastic therapeutic agent would be provided in an amount that is effective to treat neoplasia in a subject to whom the pharmaceutical composition is administered. That amount may be readily determined by the skilled artisan, as described above.

Additionally, the present invention provides methods for identifying agents for use in treating and/or preventing neoplasia. In one embodiment of the invention, the method is performed in vitro. By way of example, the method may comprise the steps of: (a) obtaining a collection of cells having aberrant PCDH expression (e.g. having at least one of PCDH8 expression decreased below normal, PCDH9 expression decreased below normal, and PCDH17 expression elevated above normal); (b) contacting a candidate agent with the cells; and (c) determining the effect, if any, of the candidate agent on the aberrant PCDH expression (e.g. on PCDH8 expression, PCDH9 expression, and/or PCDH17 expression) in the cells. The method may further comprise the steps of: (d) contacting the candidate agent with neoplastic cells having aberrant PCDH expression (e.g. having at least one of PCDH8 expression decreased below normal, PCDH9 expression decreased below normal, and PCDH17 expression elevated above normal); and (e) determining if the agent has an effect on proliferation of the neoplastic cells. In particular, the candidate agent may be assessed for its ability to act as an inhibitor to cell division or to otherwise function as an appropriate tumor-suppressing agent.

The present invention also provides an in vivo method of identifying agents for use in treating and/or preventing neoplasia. By way of example, the method may comprise the steps of: (a) obtaining an animal comprising cells having aberrant PCDH expression (e.g. having at least one of PCDH8 expression decreased below normal, PCDH9 expression decreased below normal, and PCDH17 expression elevated above normal; (b) administering a candidate agent to the animal; and (c) determining the effect, if any, of the candidate agent on the aberrant PCDH expression (e.g. on PCDH8 expression, PCDH9 expression, and/or PCDH17 expression) in the animal. The method may further comprise the steps of: (d) contacting the candidate agent with an animal comprising neoplastic cells, wherein the neoplastic cells have aberrant PCDH expression (e.g. have at least one of PCDH8 expression decreased below normal, PCDH9 expression decreased below normal, and PCDH17 expression elevated above normal); and (e) determining if the agent has an effect on proliferation of the neoplastic cells. In particular, the candidate agent may be assessed for its ability to act as an inhibitor to cell division or to otherwise function as an appropriate tumor-suppressing agent.

The present invention is further directed to agents identified by the above-described identification methods. These agents are expected to be useful in treating or preventing neoplasia in a subject. Accordingly, the present invention further provides a method for treating or preventing neoplasia in a subject, comprising administering to the subject an agent of the present invention, in an amount effective to treat or prevent the neoplasia in the subject. This amount may be readily determined by one skilled in the art. Also provided is a use of an agent of the present invention in a method of treating or preventing neoplasia.

The present invention also provides a pharmaceutical composition comprising an agent identified by one of the above-described identification methods, and a pharmaceutically-acceptable carrier. Examples of suitable pharmaceutically-acceptable carriers, and methods of preparing pharmaceutical formulations and compositions, are described above. The pharmaceutical composition of the present invention would be useful for administering to a subject an agent of the present invention, in order to treat or prevent neoplasia in the subject. In such cases, the pharmaceutical composition is administered to a subject in an amount effective to treat or prevent the neoplasia.

The present invention is described in the following Examples, which are set forth to aid in the understanding of the invention, and should not be construed to limit in any way the scope of the invention as defined in the claims which follow thereafter.

### EXAMPLES

### Example 1

### Cell Lines and Tumor Samples

UACC-812, UACC-893, MDA-MB-453, MDA-MB-175vii, MDA-MB-468, MDA-MB-361, MDA-MB-231, MDA-MB-436, MDA-MB-415, MDA-MB-330, MDA-MB-157, MDA-MB-134vi, MDA-MB-435s, ZR75-30, ZR75-1, BT-549, BT-483, T-47D, BT-474, DU4475, CAMA-1, MCF7, SK-BR-3, Hs578t, HCC38, HCC1143, HCC1187, HCC1395, HCC1428, HCC1806, HCC1937, HCC2157, HCC1500, HCC1599, HCC2218, HCC1419, HCC70, HCC202, HCC1954, HCC1569, HCC1008, LNCaP, DU145, MCF10A and PC-3 were purchased from the American Type Culture Collection (ATCC). SUM44P92, SUM52PE, SUM102PT, SUM149PT, SIJM159PT, SUM185PE, SUM225PE, SUM190PT, and SUM1315MO2 were acquired from the University of Michigan's Comprehensive Cancer Center. M2E6E7, M3E6E7, 70N, 70E6, 76N, and 76E6 were obtained from V. Band. HCC1395 and HCC1395 were obtained from Adi Gazdar. All cell lines, except MCF10A, were maintained per distributors' protocols. MCF10A were grown as described (Debnath, J., Muthuswamy, S. K. & Brugge, J. S. Morphogenesis and oncogenesis of MCF-10A mammary epithelial acini grown in three-dimensional basement membrane cultures. Methods 30, 256-68 (2003)). Breast tumor samples were from the Columbia University Tumor Bank. Female murine breast and brain tissue were from virgin and parous female mice.

### Example 2

### DNA Purification

Tissue was digested in SDS/proteinase K. DNA was extracted with phenol:chloroform:isoamyl alcohol (Invitrogen), and precipitated by NaCl/ethanol. The DNA pellet was resuspended in LoTE.

### Example 3

### Representational-Difference Analysis

Genomic subtraction was performed using a modified method of representational-difference analysis (Lisitsyn et al., Cloning the differences between two complex genomes. Science, 259:946-51, 1993). Unique sequence was identified in 18 of 150 clones. Six fragments were derived from the EBV genome. Two of the fragments mapped to chromosome 13q21 and were absent in the tumor line.

### Example 4

### RNA Isolation and RT-PCR

Cells were lysed in solution D, and RNA was separated by CsCl. RNA was resuspended in sodium acetate, and reprecipitated with ethanol. The pellet was dissolved in DEPC-treated water. RNA was primed with random hexamers, and reverse-transcribed with Superscript II reverse transcriptase (Invitrogen).

cDNA was synthesized from RNA primed with random hexamers (Amersham). Primers used for RT-PCR are below: DIAPH3: ATCTCCCTGATCAAGACTCAAT (SEQ ID NO:74, ACTGTGAGAAAGTGGAAAGTA (SEQ ID NO:75). PCDH8: TGGCGGTGTGGAAAGGACA (SEQ ID NO:75), CGGAGTGACCTGTATATGTG (SEQ ID NO:76). For reactivation studies, cells were treated with 1 μM 5-aza-deoxycytidine for 72 hours.

### Example 5

### LOH and Mutation Analysis

Markers D13S1305, D13S155, and D13S1228 were amplified from genomic DNA. A reduction in peak intensity of 50% or greater was scored as a loss. PCDHs 8 and 17 were amplified from genomic DNA using Platinum Taq (Invitrogen). Sequences were analyzed using Mutation Surveyor (SoftGenetics). The primers that were used are set forth in Tables 1 and 2.

TABLE 1

PCDH8 Amplification and Sequencing Primers

| Amplification | | Sequencing | |
|---|---|---|---|
| Primers | Sequence | Primers | Sequence |
| PCDH8-F5 | CCTTAGCCTTTCGGATCGCACT | P8SEQF1B | ATGAGTCCTGTGAGGCGT |
| | (SEQ ID NO: 1) | | (SEQ ID NO: 2) |
| PCDH8-R5 | CTCGCGTCTGCAGCTCCA | P8SEQR2 | AAGGCCAGCACGCACTGCG |
| | (SEQ ID NO: 3) | | (SEQ ID NO: 4) |
| | | P8SEQF2 | CGCAGTGCGTGCTGGCCTT |
| | | | (SEQ ID NO: 5) |
| | | P8SEQF0 | AGCCTTTCGGATCGCACT |
| | | | (SEQ ID NO: 6) |
| | | P8SEQR0 | CTGTGCGGCTCGGCCAG |
| | | | (SEQ ID NO: 7) |
| PCDH8-F6C | TTTCGCGTGGAGCTGCAGAC | P8SEQF12 | GACGCGAGCGGACGGCG |
| | (SEQ ID NO: 8) | | (SEQ ID NO: 9) |
| PCDH8-R6D | TTGTCGTTCTCGTCGCCCAC | P8SEQE4 | CGCGATCAGGCCGCCT |
| | (SEQ ID NO: 10) | | (SEQ ID NO: 11) |
| | | P8SEQF5 | TGCCACTTCGCTGGTGCC |
| | | | (SEQ ID NO: 12) |
| PCDH8- | GTGCGCTGCGCCCTCTATG | P8SEQF13 | GCCCTCTATGGGCACGAG |
| BKUP3F | (SEQ ID NO: 13) | | (SEQ ID NO: 14) |
| PCDH8- | CTGTTACCACGAAGCTGACA | P8SEQF6 | CCGGTCTATGAGGTGTCG |
| BKUP3R | (SEQ ID NO: 15) | | (SEQ ID NO: 16) |
| | | P8SEQF7 | CTGGACCAGAACGACCATGC |
| | | | (SEQ ID NO: 17) |
| | | P8SEQR8 | ACAGTTGCGGTGGTGGT |
| | | | (SEQ ID NO: 18) |
| PCDH8-F4 | CAGGAGCCGCGCGAAG | PCDH8-F4 | CAGGAGCCGCGCGAAG |
| | (SEQ ID NO: 19) | | (SEQ ID NO: 20) |
| PCDH8-R4 | CTGCAGCAGGGAATAGGG | P8SEQF14 | GAAGCCTTCGCCATCGGCCG |
| | (SEQ ID NO: 21) | | (SEQ ID NO: 22) |
| | | PCDH8-R4 | CTGCAGCAGGGAATAGGG |
| | | | (SEQ ID NO: 23) |
| | | P8SEQR11 | CGGGAGCCGAGGCTCCGCC |
| | | | (SEQ ID NO: 24) |
| PCDH8-X2F | GGTAGGGTGGGTGCATTCAGGTAG | 13-50F | GTAGACAACCTGTCGCACTT |
| | (SEQ ID NO: 25) | | (SEQ ID NO: 26) |
| PCDH8-X2R | GCGGGAAAGAAGAGTCCTCACCAC | P8SEQX2R | CTCACCACTCTGCATGTGGT |
| | (SEQ ID NO: 27) | | (SEQ ID NO: 28) |
| PCDH8-X3F | CTGACCCAGGCTGACATGCTGAAG | P8SEQX3F | GGAAGCCTGAATATGTATAA |
| | (SEQ ID NO: 29) | | (SEQ ID NO: 30) |
| PCDH8-X3R | TTAGGGGCTTCTCGGAGTGACCTG | P8R2C | CGGAGTGACCTGTATATGTG |
| | (SEQ ID NO: 31) | | (SEQ ID NO: 32) |

TABLE 2

PCDH17 Amplification and Sequencing Primers

| Amplification | | Sequencing | |
|---|---|---|---|
| Primers | Sequence | Primers | Sequence |
| P17-F20 | TGGCTCCTCCAGTCCGATTG | P17SEQF20 | CTCCTGCCCCCACCTTACAG |
| | (SEQ ID NO: 33) | | (SEQ ID NO: 34) |
| P17-R20 | TAGGTGCGGAGCCCATTCTC | P17SEQR20 | GGCGTCGGGGTCATGTGCGC |
| | (SEQ ID NO: 35) | | (SEQ ID NO: 36) |
| P17-F28 | ATCCAGGACATCAACGACAACG | P17SEQF28 | CGCGCCCTCCTTCTCCTCGG |
| | (SEQ ID NO: 37) | | (SEQ ID NO: 38) |
| P17-R28 | CCCTTCACACGGATTAGGCC | P17SEQR28 | CGGTCTTGGGGTCGATGG |
| | (SEQ ID NO: 39) | | (SEQ ID NO: 40) |
| P17-F22 | CCATCGACCCCAAGACCGGCCTAAT | P17SEQF22 | TGAAGGGCAATCTGGACTAT |
| | (SEQ ID NO: 41) | | (SEQ ID NO: 42) |
| P17-R22 | TAGAGCCCTTTGGTGAACCGAGGCG | P17SEQR22 | TTGTCGTTCTCTGTCTAGAAT |
| | (SEQ ID NO: 43) | | (SEQ ID NO: 44) |
| P17-F23 | CCAGTCGTTCGCGATCAAG | P17SEQF23 | ATTCTAGACGAGAACGAC |
| | (SEQ ID NO: 45) | | (SEQ ID NO: 46) |
| P17-R23 | GTCGATCTCAAACAGGTGGTCG | P17SEQR23 | GTTGCCGTCCACGATCTCGT |
| | (SEQ ID NO: 47) | | (SEQ ID NO: 48) |
| P17-F24 | CGTCTCACCTACGAGATCGTG | P17SEQF24 | GGACGGCAACGACGACCACC |
| | (SEQ ID NO: 49) | | (SEQ ID NO: 50) |
| P17-R24 | GGCTGCTCACCACGTTCATG | P17SEQR24 | GTTCATGGCGTTCCTCTCC |
| | (SEQ ID NO: 51) | | (SEQ ID NO: 52) |
| P17-F25 | AGGGCAAGAAGAAGAAGATCAACAA | P17SEQF25 | ATGATATCATGCTGGTGCAG |
| | (SEQ ID NO: 53) | | (SEQ ID NO: 54) |
| P17-R25 | CATTTTTTTCTGCTCTGGGAATTCC | P17SEQR25 | CAAAGGTTTCAGGACAGCT |
| | (SEQ ID NO: 55) | | (SEQ ID NO: 56) |
| P17X2-3F2 | TCCACTAGGAAATCTTGTTGCTCTG | P17SEQX2- | ATTTGTCCAGTGTACAACTA |
| | (SEQ ID NO: 57) | 3F | (SEQ ID NO: 58) |
| P17X2-3R | CCATTCCACTGTGATAAACTGCAAC | P17SEQX2- | ACTGCTTAAGCTTTACACTT |
| | (SEQ ID NO: 59) | 3R | (SEQ ID NO: 60) |
| P17X4F2 | GGATTTGCTGTTTTCTCT | P17SEQX4F | GTTTTGCAGAACCAGAAG |
| | (SEQ ID NO: 61) | | (SEQ ID NO: 62) |
| P17X4R2 | ACCAGGAGGGATCATTTT | P17SEQX4R3 | CAAGAAAATGCCAATGCCTT |
| | (SEQ ID NO: 63) | | (SEQ ID NO: 64) |
| | | P17SEQX4F2 | GTGCATCGAGCCTTGCACC |
| | | | (SEQ ID NO: 65) |
| | | P17SEQX4R2 | AGCCAGGGCTCCTGGTTTTG |
| | | | (SEQ ID NO: 66) |

### Example 6

### Southern Blots

5-10 micrograms of genomic DNA were digested with RsaI alone or with RsaI in combination with HpaII, MspI, or CfoI. The digest was transferred to nylon membranes (Nytran), cross-linked, and baked. Membranes were hybridized with ³²P-labeled probes in UltraHyb (Ambion), washed, and exposed to film. Probes were generated by PCR amplification from genomic DNA, and labeled randomly using Klenow polymerase (USB) and ³²P-dCTP (Amersham).

PCR primers are as follows:

| | |
|---|---|
| 13q21 probe: | |
| 13q21F: | AGGCTTTTGAGTTCAAGGTG, |

(SEQ ID NO: 77)

| | |
|---|---|
| 13q21R: | GTAAGTCTCAGTCTCAACA; |
| PCDH8 probe: | |

(SEQ ID NO: 78)

| | |
|---|---|
| PCDH8-CpG-F3: | AGAGGCTATTCCAGGCACCG, |

(SEQ ID NO: 79)

| | |
|---|---|
| PCDH8-CpG-R3: | CTCTCGGAATCACGCTCTTTG; |
| ANKRD3 probe: | |

(SEQ ID NO: 80)

| | |
|---|---|
| ANKRD3-F: | GGACGACCTACGGAAGTGAC, |

(SEQ ID NO: 81)

| | |
|---|---|
| ANKRD3-R: | CTAACTCCACTCACAAAGCC. |

### Example 7

### In Situ Hybridization

Tissue was fixed in 4% paraformaldehyde overnight at 4° C., and dehydrated in 30% sucrose, 15-micron cryosections were cut and dried. Sections were fixed in 4% paraformaldehyde, digested in proteinase K, re-fixed, and acetylated. Sections were hybridized with DIG-labeled cRNA probes (for example from mouse clone ID 3813893), and then incubated with anti-DIG-AP antibody (Roche). Alkaline phosphatase activity was visualized with vector NBT/BCIP. Probes were transcribed with T7, SP6, or T3 RNA polymerases from linearized EST clones, and labeled with DIG-NTPs.

### Example 8

### 5-Aza-Deoxycytidine Treatment

Cells were plated to 20% confluence, and treated with fresh 1 micromolar 5-aza-deoxycytidine for 72 h. RNA and cDNA were prepared from the cells and subjected to PCR.

Summarized below are results obtained by the inventors in connection with the experiments of Examples 1-8 above.

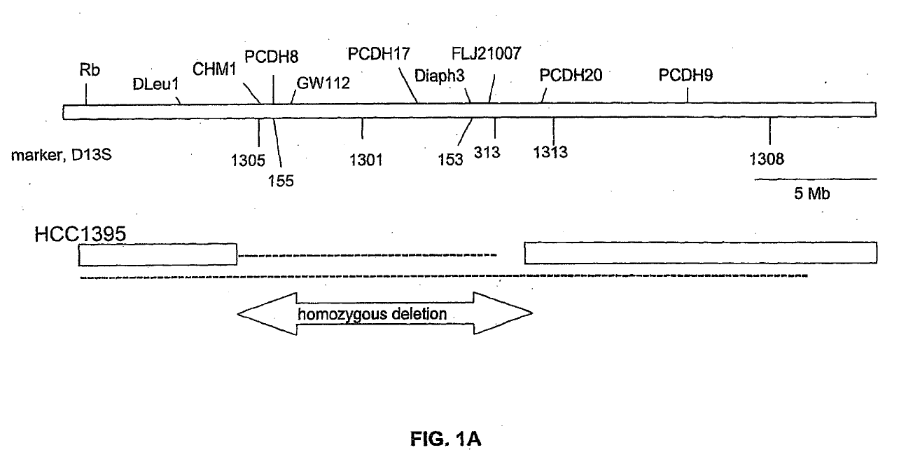

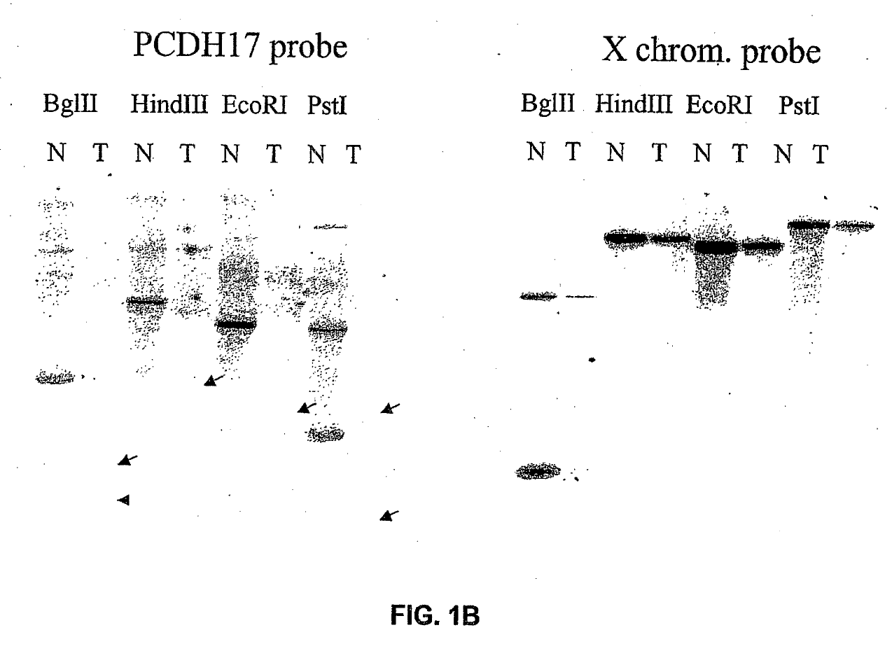

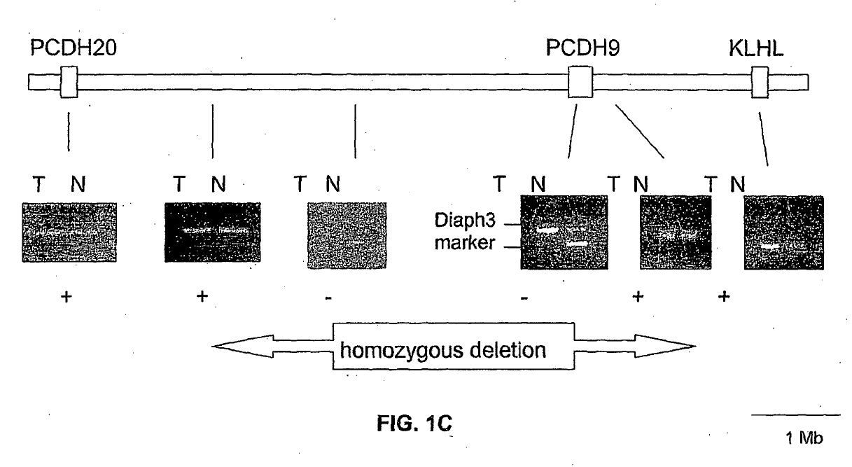

Representational difference analysis and genomic subtraction revealed a homozygous deletion of a portion of chromosome 13q in the breast tumor cell line HCC1395 (FIG. 1A). Deletion of markers D13S1305, D13S155, D13S1301, D13S153, and D13S313 was observed in HCC1395. This included several genes, which are highlighted in red in the figure. Southern-blot analysis (FIG. 1B) then confirmed that protocadherin 17 (PCDH17) was absent from HCC1395, but present in a normal lymphoblastoid line from the same patient.

Since protocadherins 17 and 20 were missing in HCC1395, the inventors screened protocadherins 17, 8, 9, and 20 for homozygous deletions in 88 human breast tumor lines and xenografts. From this screen, an additional homozygous deletion was identified in PCDH9 in the breast tumor line, MDA-MB-436 (FIG. 1C). Duplex PCR revealed a deletion of PCDH9 in MDA-MB-436. The deletion boundary was mapped by PCR, and PCDH9 was the only gene mapped in this deletion.

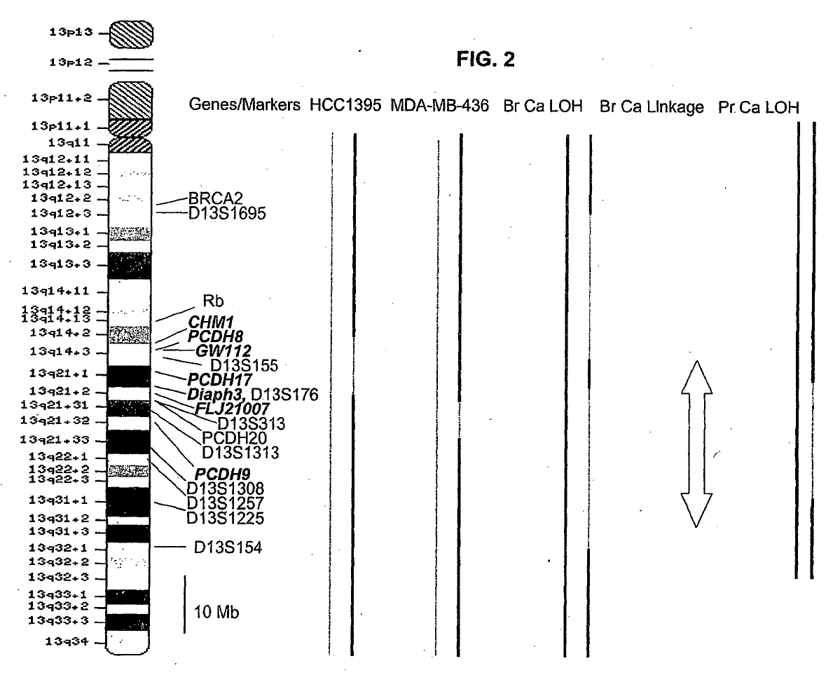

Homozygous deletions, loss of heterozygosity (LOH), and linkage data in hereditary breast cancer are summarized in FIG. 2. All of these data implicate one or more of the chromosome-13 protocadherins as candidate genes for breast cancer.

The deletions overlap with regions of LOH found in breast and prostate cancer. Furthermore, they fall within the linked interval found in hereditary breast cancers that are free of BRCA1 and BRCA2 mutations. (For breast cancer breast cancer LOH, see Eiriksdottir et al., Mapping loss of heterozygosity at chromosome 13q: loss at 13q12-q13 is associated with breast tumour progression and poor prognosis. Eur. J. Cancer, 34(13):2076-81, 1998; for breast cancer linkage, see Kainu et al., Somatic deletions in hereditary breast cancers implicate 13q21 as a putative novel breast cancer susceptibility locus. Proc. Natl. Acad. Sci. USA, 97(17):9603-08, 2000; and for prostate cancer LOH, see Hyytinen et al., Three distinct regions of allelic loss at 13q14, 13q21-22, and 13q33 in prostate cancer. Genes Chromosomes Cancer, 25(2):108-14, 1999). A protocadherin cluster partly lies within the deletions. In HCC1395, PCDHs 8 and 17 are deleted; in MDA-MB-436, PCDH9 is deleted.

Invasive ductal carcinoma of the breast evolves from epithelial cells of breast ducts. These ducts are composed of luminal, basal, and myoopithelial components. The breast acinus or duct is formed by 2 layers of cells. A layer of polarized, cuboidal, luminal cells lines the lumen; a second layer of cells, consisting of basal and myoepithelial cells, is subjacent to the luminal cells. Most breast tumors arise from the luminal epithelium.

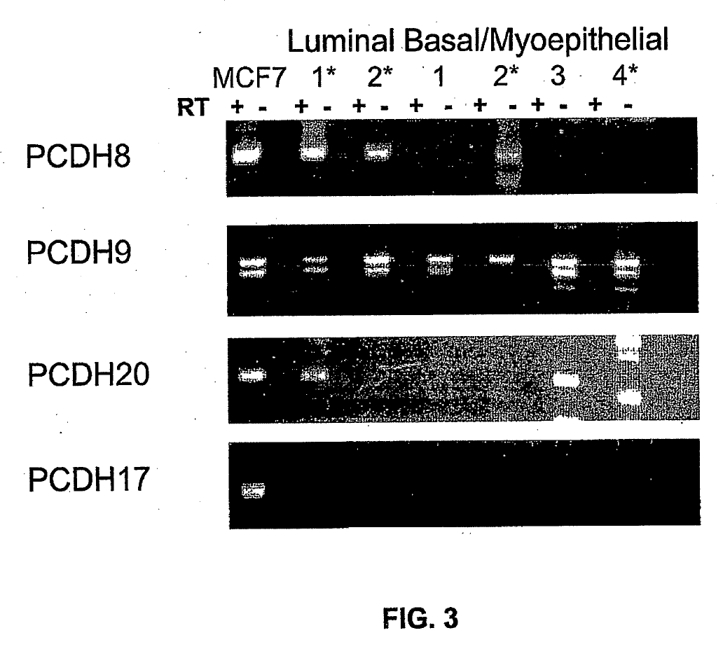

Examination of the expression of protocadherins in cultured breast epithelial cells demonstrated that protocadherin 9 was expressed in both luminal and basal/ myoopithelial cell types, while protocadherin 8 was restricted to the luminal cells (FIG. 3). Interestingly, there was no evidence of protocadherin 17 expression in the normal cultured cells. Protocadherin 20 was expressed in only 1 luminal and 1 basal culture.

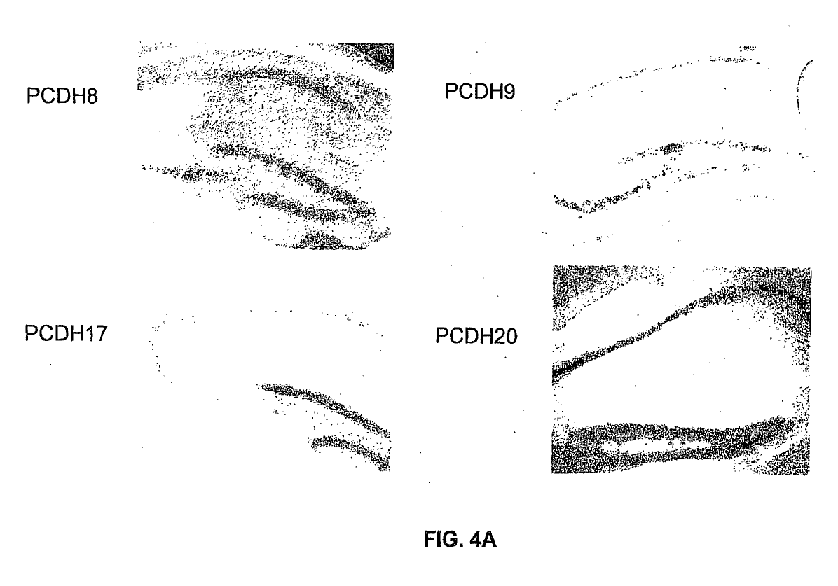

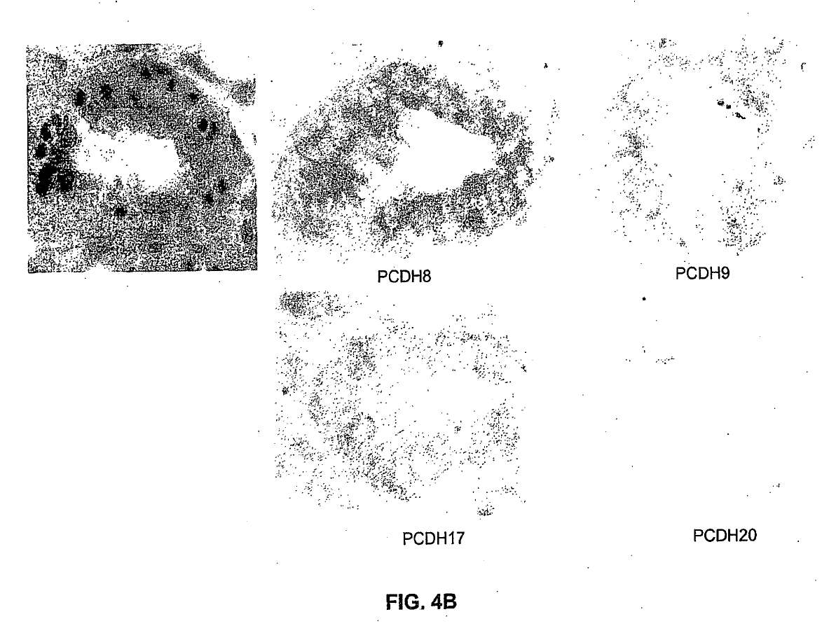

In situ probes for the 4 protocadherins were prepared. The probes were then hybridized to normal mouse brain sections. In situ hybridization revealed that all four probes were strongly expressed in the hippocampus (FIG. 4A). Thus, the 13q cluster of protocadherins is syntenic in mice.

The same in situ probes were then annealed to frozen sections of wild-type mouse mammary tissue. In situ hybridization revealed different expression patterns for PCDHs 8, 9, 17, and 20 in mouse breast tissue. Specifically, PCDH8 was expressed in luminal cells; PCDH9 was expressed in luminal cells, and possibly in basal/myoepithelial cells; and PCDH20 was expressed in luminal cells, and possibly in basal/myoepithelial cells. PCDH17 expression was not detected in breast epithelial cells.

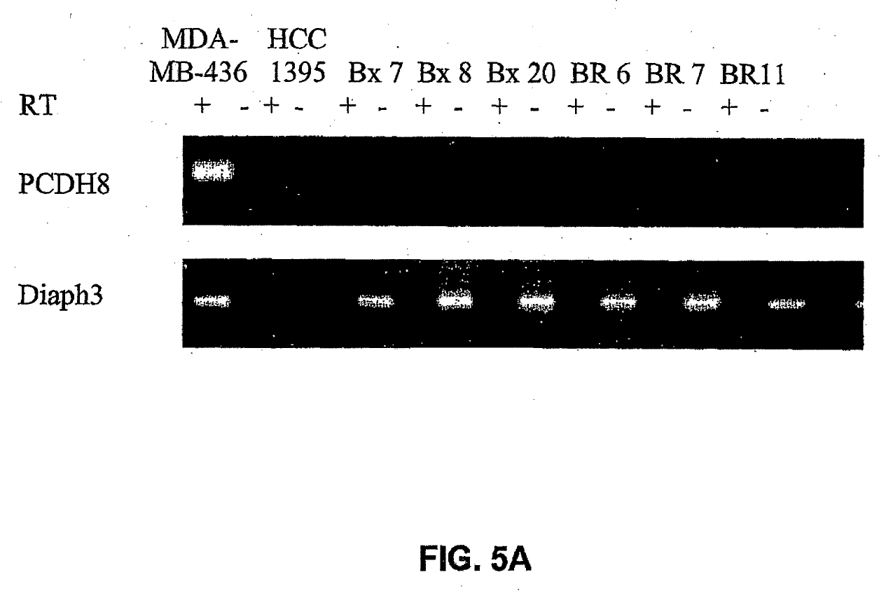

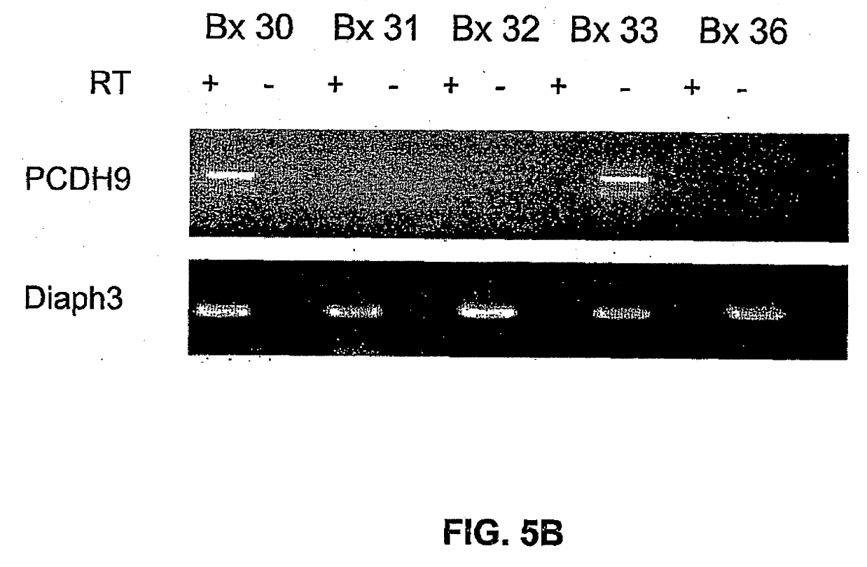

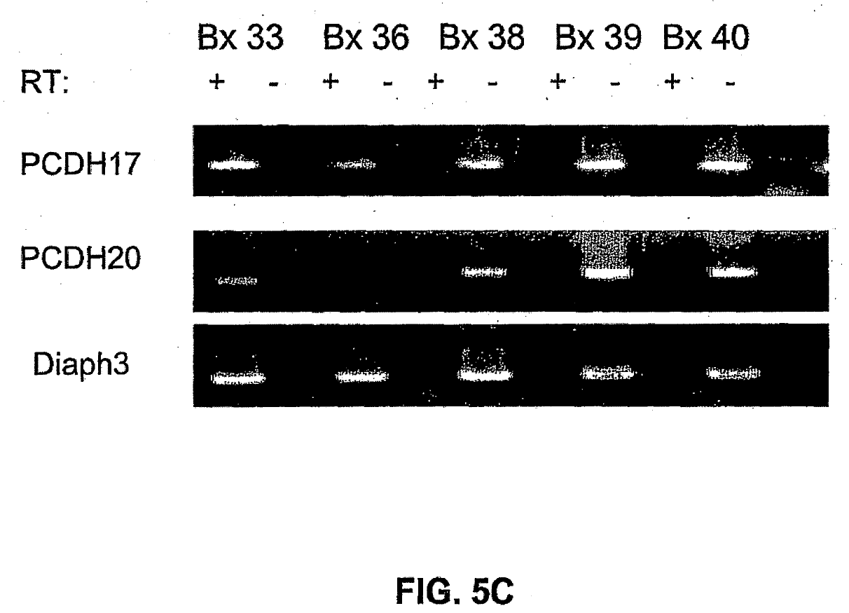

In contrast to the expression of protocadherin 8 seen in normal breast tissue, lack of PCDH8 expression was frequently observed in breast tumor xenografts and primary breast tumors (FIG. 5A). A similar reduction in the expression of protocadherin 9 was also observed in breast tumor xenografts (FIG. 5B). However, expression of protocadherin 17 and was frequently present in breast tumor xenografts (FIG. 5C).

As shown in Table 3, protocadherin 9 expression was reduced in about 35% of breast tumor cell lines and xenografts, and in 2 out of three prostate tumor cell lines. Reduced expression was observed in only 10% of primary tumors; this may reflect contamination from the high expression seen in normal ductal cells. Loss of protocadherin 8 expression occurred in 18% of cell lines, and in 39% of tumor xenografts and primary tumors. Loss of protocadherin 20 expression was less common, occurring in 8%, 19%, and 5% of tumor lines, xenografts, and primary biopsies, respectively. Since protocadherins 8, 9, and 20 are normally expressed in breast tissue, these data suggest that their genes were silenced during tumor evolution. Interestingly, this was also the case in 2 prostate cancer cell lines. The inventors observed that 90-100% of tumor samples expressed the PCDH17 gene; this gene is silent in normal breast epithelium.

TABLE 3
Expression of PCDHs 9, 8, 20, and 17 in Breast and Prostate Cancer

| | | | |
|---|---|---|---|
| PCDH9 | PCDH8 | PCDH20 | PCDH17 |

Breast Cancer

| | | | | | | | | |
|---|---|---|---|---|---|---|---|---|
| Cell Lines | 28/41 | (68.3)* | 36/44 | (81.8) | 38/41 | (92.7) | 37/41 | (90.2) |
| Xenografts | 20/31 | (64.5) | 19/31 | (61.3) | 25/31 | (80.6) | 31/31 | (100.0) |
| Primary Tumors | 37/41 | (90.2) | 25/41 | (61.0) | 37/39 | (94.9) | 41/41 | (100.0) |

Prostate Cancer

| | | | | | | | | |
|---|---|---|---|---|---|---|---|---|
| Cell Lines | 1/3 | (33.3) | 3/3 | (100.0) | 3/3 | (100.0) | 3/3 | (100.0) |

*number of samples expressing a gene/total number of samples (% of tumors)

The data shown in Table 3 suggest that a switch in the pattern of protocadherin expression occurs in breast cancer development, where PCDH17 is turned on, and PCDHs 8 and 9 are silenced. Little change occurs for protocadherin 20.

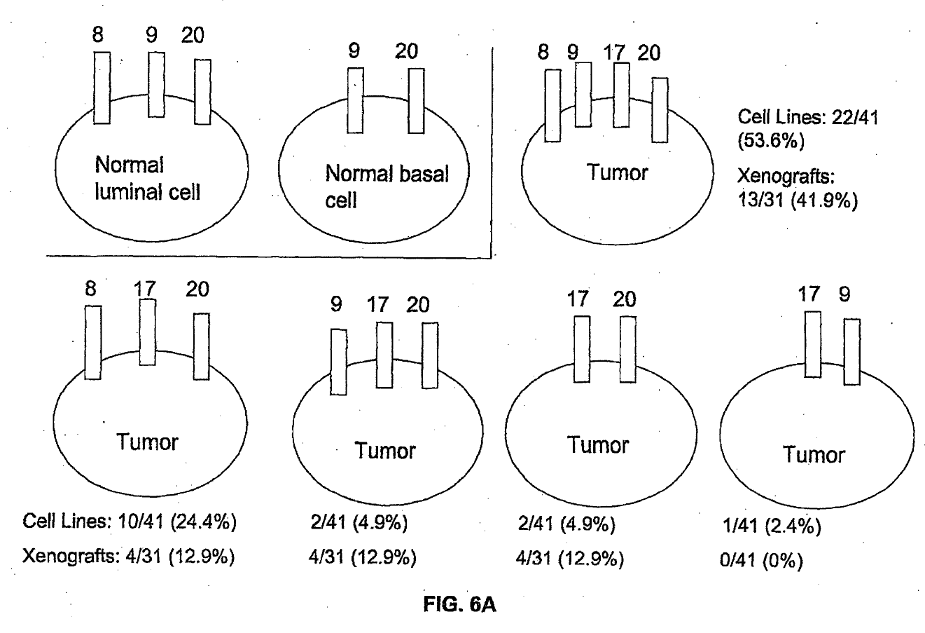

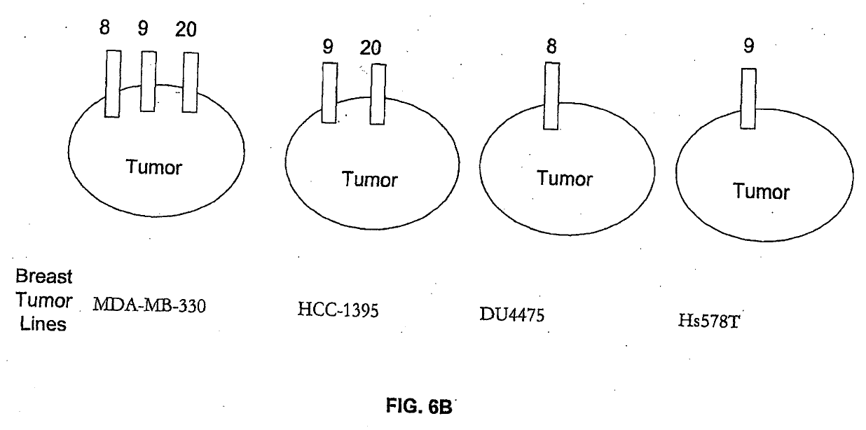

Patterns of altered PCDH expression in tumors are also shown in FIG. 6A. Protocadherin 17 was not expressed in normal ductal epithelium, but protocadherins 8, 9, and were. In tumors, PCDH17 was turned on in nearly all cases, while PCDHs 8, 9, and 20 were silenced to varying extents. More specifically, PCDH9 and PCDH20 were expressed in normal basal cells, while PCDHs 8, 9, and 20 were expressed in normal luminal cells. The most common protocadherin expression pattern found in breast tumors was one in which every protocadherin in this cluster was expressed. Other common expression patterns were ones in which PCDH17 was turned on, and PCDH8 or PCD119 was turned off. Occasionally, PCDH17 was turned on, and both PCDHs 8 and 9 were turned off.

MDA-MB-330 was the only breast tumor line or xenograft with a normal pattern of 13q protocadherin expression (FIG. 6B). The other 3 cell lines that lacked expression of protocadherin 17 also lost expression of one or more of the other genes in the cluster. Rarely, PCDH17 remained off, but this was often accompanied by the downregulation of PCDHs 8 or 9. Overall, these data show that tumors tend to silence protocadherins 8 and 9, and turn on protocadherin 17.

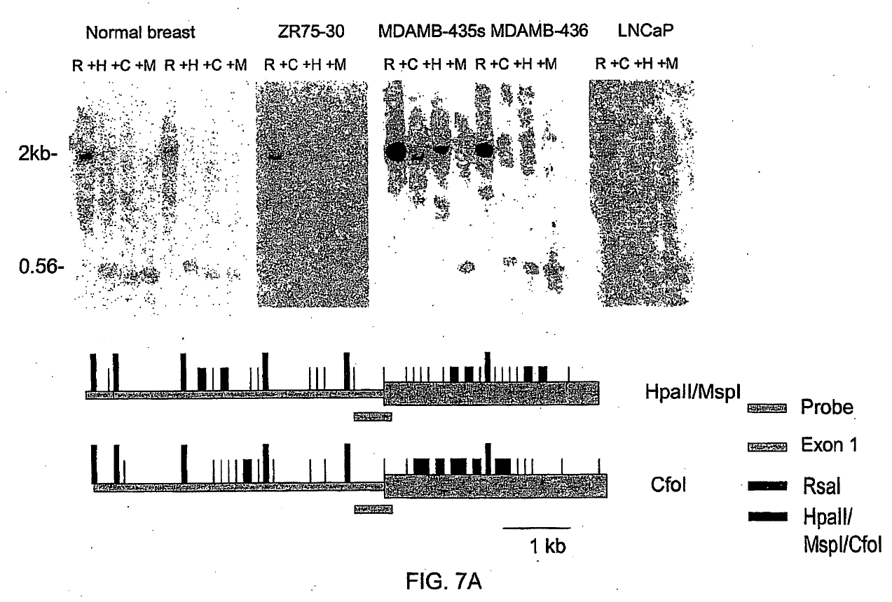

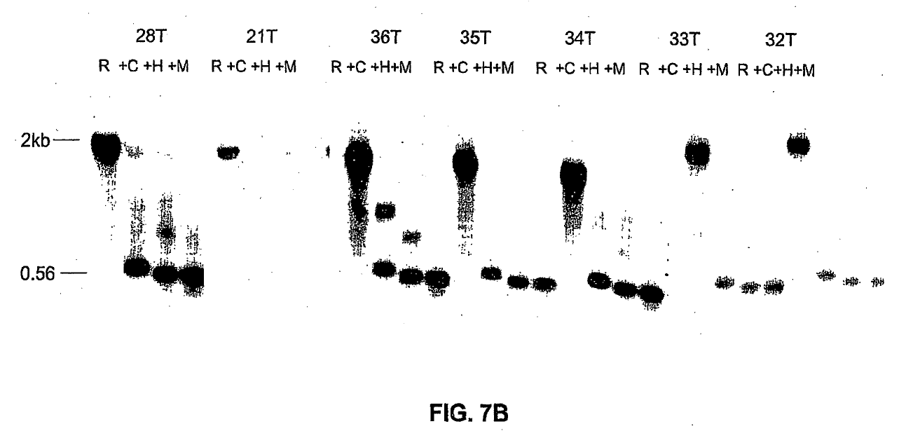

As demonstrated by Southern-blot analysis, tumor cell lines that failed to express protocadherins—such as ZR75-30 and MDA-MB-435s—show strong methylation of the PCDH8 promoter (FIG. 7A). Normal breast and tumor lines—MDA-MB-436 and LNCaP, respectively, both of which express protocadherin 8—showed no evidence of methylation.

Primary tumors were then screened for evidence of methylation (FIG. 7B). Southern-blot analysis showed that the PCDH8 promoter was methylated in primary breast tumors, 28T, 21T, 36T, 35T, and 34T. Two tumors, 28T and 21T, showed evidence of full methylation of some alleles within the tumor. Tumors 36T, 35T, and 34T showed evidence of partial methylation. No methylation was detected in 33T and 32T.

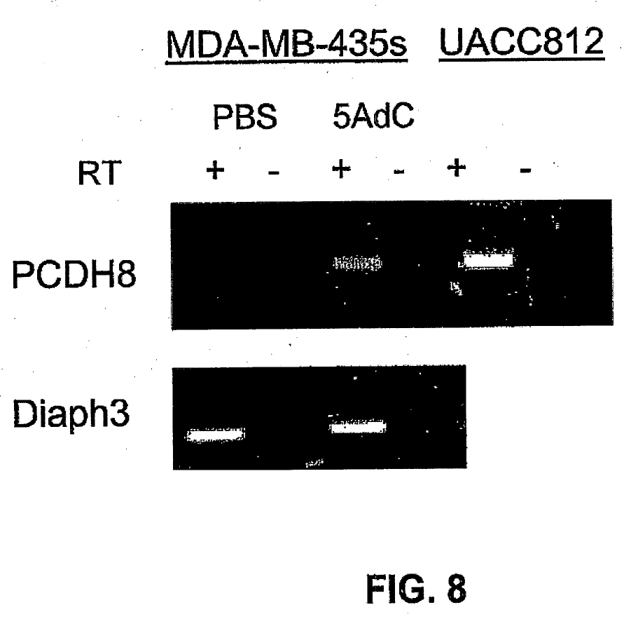

To demonstrate that promoter methylation could be responsible for gene silencing of protocadherin 8, the inventors treated MDA-MB-435s cells with the DNA methyltransferase inhibitor, 5-aza-deoxycytidine. PCDH8 expression was restored in MDA-MB-435 cells treated with 5-aza-deoxycytidine (FIG. 8).

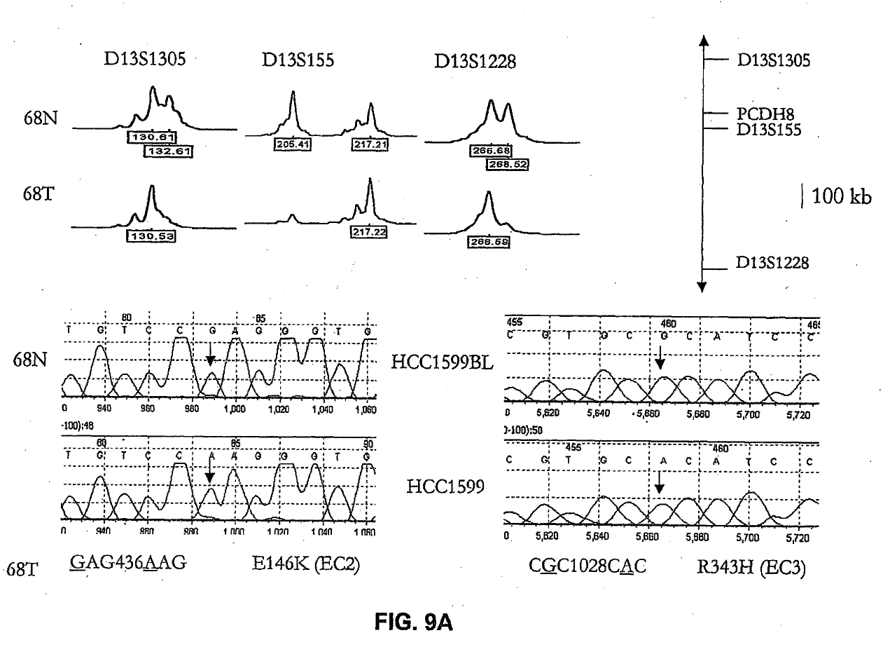

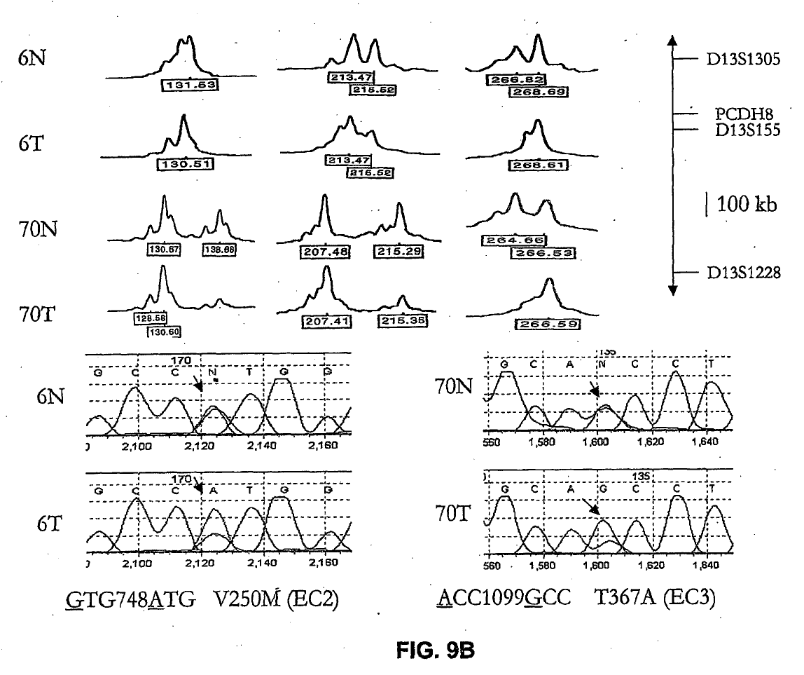

The inventors then examined the possibility that protocadherin 8 could be mutated in breast cancer. Seventy-five cases of breast cancer were screened for loss of heterozygosity. Of these, 29 exhibited loss of one allele in the vicinity of protocadherin 8 (39%). The exons of protocadherin 8 were amplified and sequenced in 28 of these tumors, and in 23 cell lines that expressed protocadherin 8 by RT-PCR. Somatic mutations were found in two cases. Both occurred in the extracellular domain, and fell within the cadherin repeats. Breast tumor 68T exhibited loss of heterozygosity (LOH) at markers D13S 1305, D13S155, and D13S1228 (FIG. 9A). A mutation of G436A was predicted to change aspartate⁴⁶ to lysine. Breast cancer cell line HCC 1599 was found to harbor mutation G1028A, changing arginine³⁴³ to histidine.

Germline mutations were also found in two cases (FIG. 9B). For both of these cases, loss of the wild-type allele occurred in the tumor. Breast tumors 6T and 70T showed LOH at markers D13S1305, D13S155, and D13S1228. Tumor 6T showed a G748A mutation that should change valine²⁵⁰ to alanine. Tumor 70T showed an A1099G mutation that should change threonine³⁶⁷ to alanine. Both tumors have lost the wild-type allele of protocadherin 8.

The mutation data for PCDH8 are summarized in Table 4.

TABLE 4
Summary of PCDH8 Mutation Data

| Sample | Mutation | Predicted Effect | Somatic/Germline | Ethnicity |
|---|---|---|---|---|
| HCC1395 | homologous | no protein | somatic | Caucasian |
| | deletion | | | |
| 68T | GAG436AAG | E146K (EC2) | somatic | Caucasian |
| HCC1599 | CGC1028CAC | R343H (EC3) | somatic | Caucasian |
| 70T | C-1T | dec translation/ | germline | African |
| | | novel protein | | American |
| 6T | GTG748ATG | V250M (EC3) | germline | Caucasian |
| T47D | GTG748ATG | V250M (EC3) | ? | ? |
| 70T | ACC1099GCC | T367A (EC4) | germline | African |
| | | | | American |
| 55T | ACC1099GCC | T367A (EC4) | germline | Caucasian |
| UACC-893 | ACC1099GCC | T367A (EC4) | ? | Caucasian |
| HCC1008 | CTG2015CAG | L672Q (EC6) | germline | African |
| | | | | American |
| HCC2157 | CTG2015CAG | L672Q (EC6) | ? | African |
| | | | | American |
| 3T | CTG2015CAG | L672Q (EC6) | germline | African |
| | | | | American |

*total number of primary tumors = 28; total number of cell lines = 23

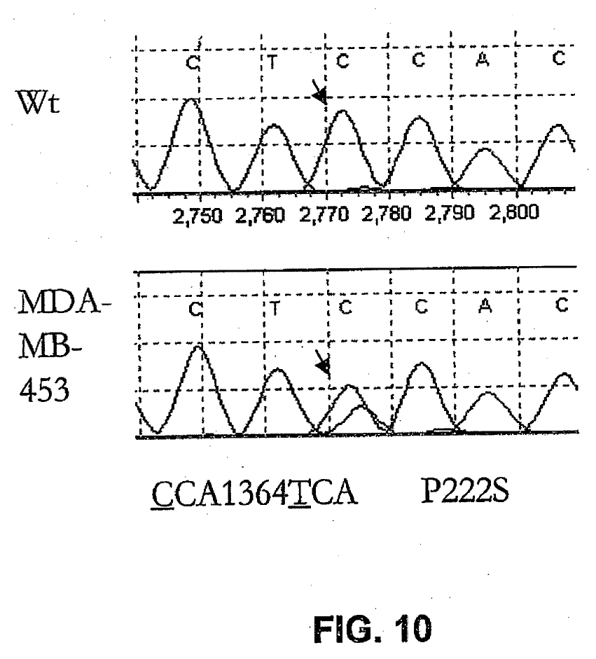

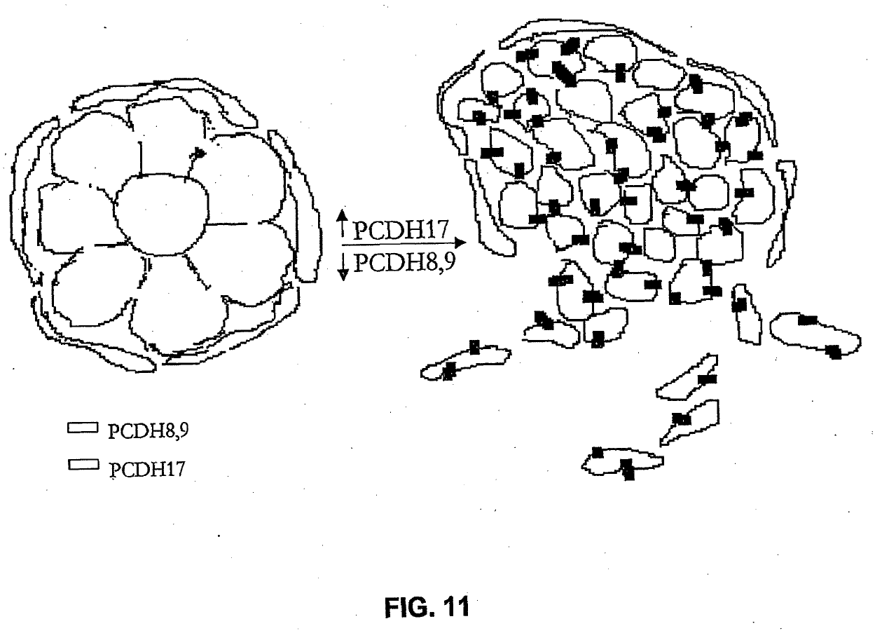

Because protocadherin 17 was abnormally activated in nearly all breast tumor samples tested, the inventors performed amplification and sequencing of protocadherin 17 in 22 tumors and in 21 cell lines. A missense mutation was identified at amino acid 222 (P222S). This mutation was in cadherin domain 2 (FIG. 10). Protocadherin mutations are uncommon, but do occur. The data in Table 5 suggest that overexpression of wild-type protein is sufficient to contribute to the tumor phenotype. However, the mutations shown above may potentiate the activity of wild-type protocadherin 17.

TABLE 5
Summary of PCDH17 Mutations

| | | Predicted | Somatic/ | |
|---|---|---|---|---|
| Sample | Mutation | Effect | Germline | Ethnicity |
| MDA-MB-43 | CCA1364TCA | P222S (EC2) | ? | Caucasian |
| IT | GAG2899GAT | E733D | germline | ? |
| | | (intra- | | |
| | | cellular) | | |

*total number of primary tumors = 22, total number of cell lines = 21

The possible results of the deregulation of protocadherins are shown in FIG. 11. The model shows expression of PCDHs 8 and 9 in normal luminal epithelial cells within a breast duct. The expression of PCDH17, and loss of PCDHs 8 and/or 9, may lead to cellular transformation. Altered expression of protocadherins in the 13q cluster may change adhesive or polar properties of breast cells. The cells may subsequently undergo an epithelial-to-mesenchymal transition. The misexpression of protocadherins may facilitate proliferation, invasion, and metastasis.

The inventors' model suggests that activation of protocadherin 17, and inhibition of protocadherins 8 and 9, contribute to tumor formation. Since nearly all samples have abnormal patterns of expression, it is believed that protocadherins 8 and 9 regulate the normal morphology of ductal cells through cell-cell communication. When their expression is lost, and protocadherin 17 is activated, the tissue-organizing signals are lost, and cells no longer respond to each other appropriately. This can lead to inappropriate migration, proliferation, and apoptosis.

### Example 9

### PCDH8 Expression in Breast Cancer

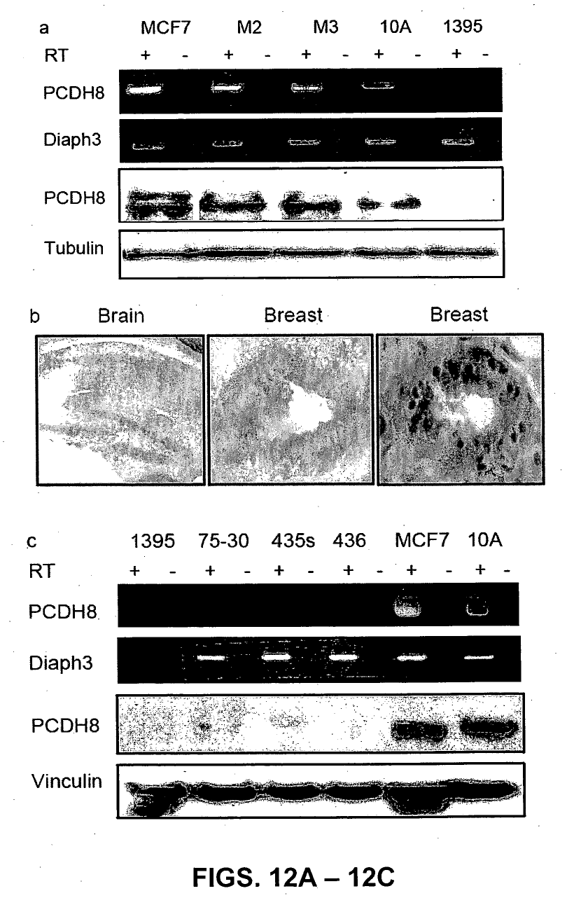

mRNA and protein expression of PCDH8 was detected in two breast luminal epithelial cells lines (M2 and M3), a spontaneously immortalized breast epithelial line, MCF10A (10A), and the breast tumor line MCF7 but not in HCC1395 the line with the homozygous deletion (FIG. 12A). (Wazer, D. E., Liu, X. L., Chu, Q., Gao, Q. & Band, V. Immortalization of distinct human mammary epithelial cell types by human papilloma virus 16 E6 or E7. Proc Natl Acad Sci USA 92, 3687-91 (1995)). To determine whether PCDH8 mRNA was produced in mammary epithelium, an in situ hybridization probe was designed and mouse PCDH8 message was found to be expressed in mouse mammary ducts and brain (FIG. 12B).

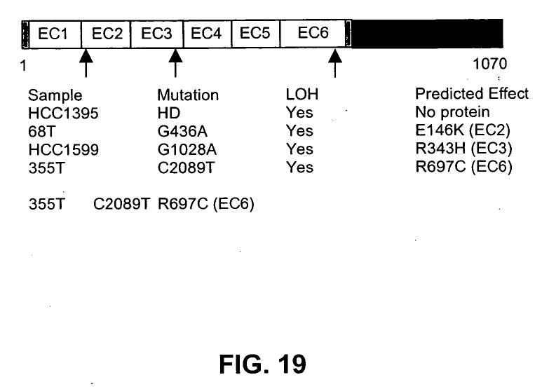

To look for changes in PCDH8 expression in breast cancer, a panel of 85 cancer cell lines and tumor biopsies were screened for PCDH8 message. As shown in FIG. 12C, PCDH8 was not detected in ZR75-30 (75-30), MDA-MB-435s (435s), or MDA-MB-436 (436). The overall frequency of PCDH8 downregulation was 32% in tumors and 18% in cell lines. (FIGS. 12C and 19). In addition, tumor cell lines such as ZR75-30, MDA-MB-435s, and MDA-MB-436 that exhibited no message for PCDH8 also expressed little to no protein (FIG. 12C).

To determine whether PCDH8 is a tumor suppressor gene, 116 breast tumors as well as 21 breast tumor cell lines were screened for mutations. In a subset of cases, LOH was screened for, and was present in 39% of cases. Three cancer-specific somatic mutations were found that were all associated with loss of the wild type allele.

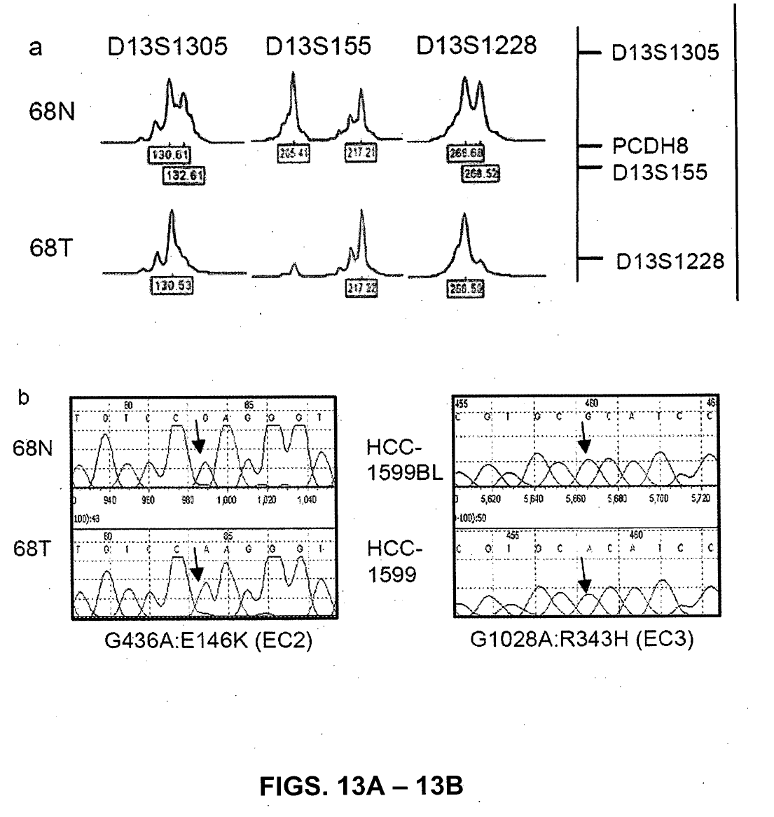

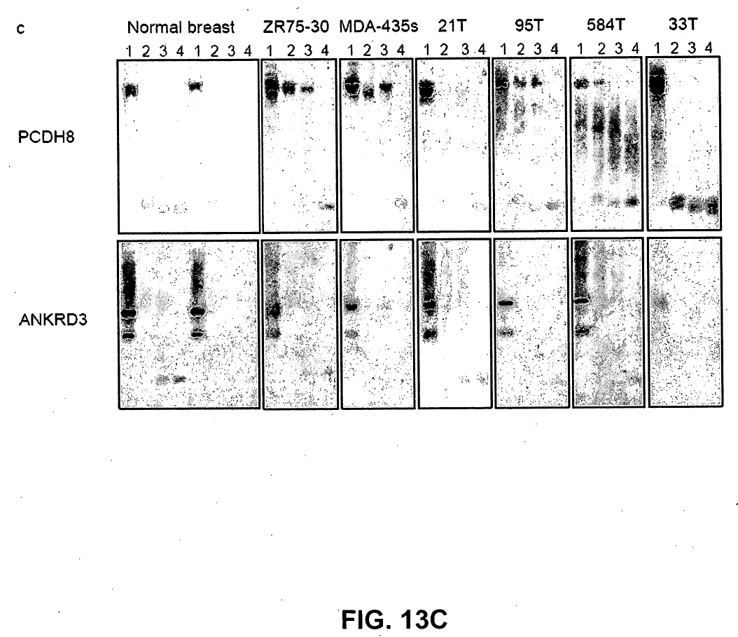

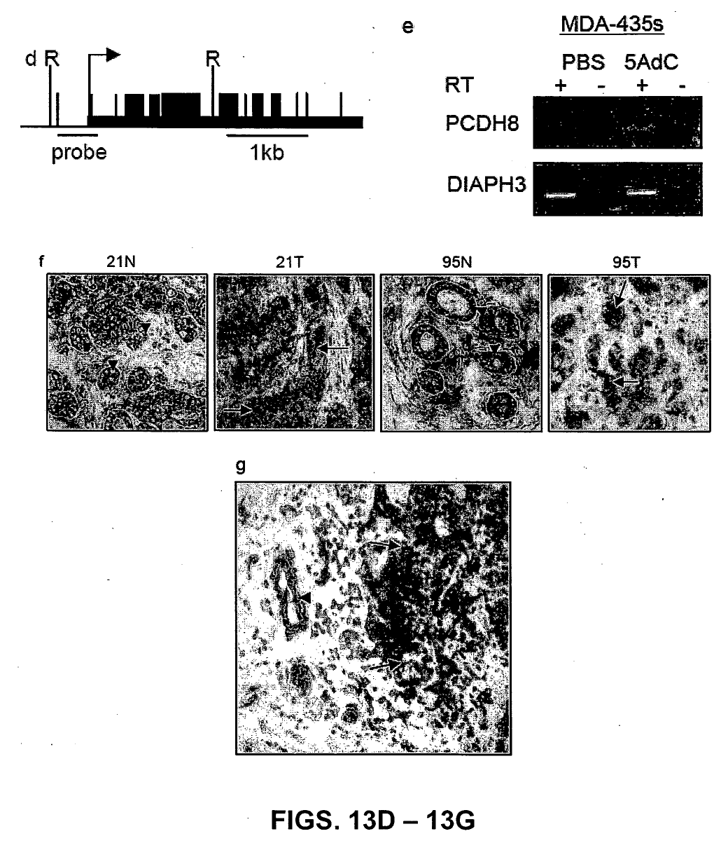

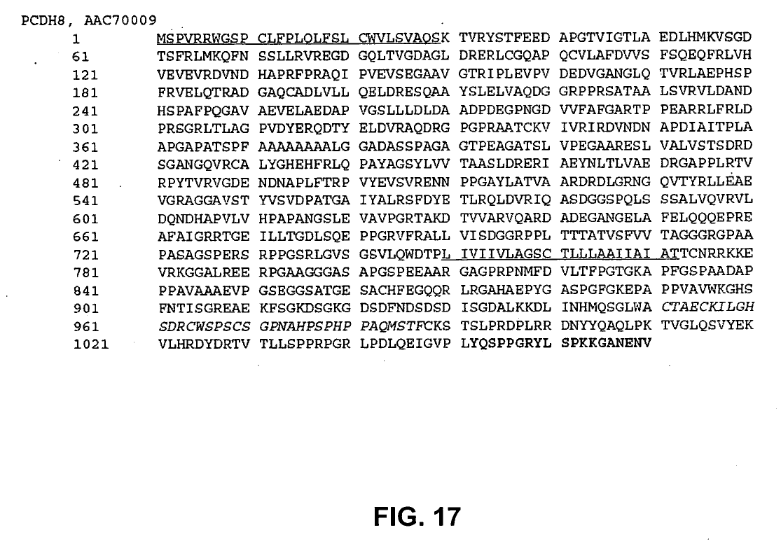

The PCDH8 gene is predicted to encode an open reading frame with a signal peptide sequence, 6 extracellular cadherin repeats (EC), a transmembrane domain, and a cytoplasmic tail (FIGS. 17 and 19). In one tumor biopsy, a G436A:E146K mutation was found in EC2; in another tumor biopsy, a C2089T:R697C mutation was found in EC6; and in HCC1599, a G1028A:R343H mutation was found in EC3 (FIGS. 13A, 13B and 19).

The genetic changes in PCDH8 found in the breast cancer samples are consistent with a tumor suppressor. All of the missense changes clustered in the cadherin repeats, showing that they may disrupt adhesive function. Alignment of the E146K mutation to the analogous glutamic acid residue in C-cadherin predicts that it coordinates calcium ions, a function that is required for proper adhesive function (Shapiro, L. et al. Structural basis of cell-cell adhesion by cadherins. Nature 374, 327-37 (1995); Nagar, B., Overduin, M., Ikura, M. & Rini, J. M. Structural basis of calcium-induced E-cadherin rigidification and dimerization. Nature 380, 360-4 (1996); Boggon, T. J. et al. C-cadherin ectodomain structure and implications for cell adhesion mechanisms. Science 296, 1308-13 (2002)).

### Example 10

### Methylation of PCDH8 CpG Island

To determine the mechanism of PCDH8 silencing seen in some breast cancer cases, PCDH8 CpG island methylation was assessed by Southern blot. The PCDH8 CpG island was not methylated in normal breast (FIGS. 13C and 13D). However, methylation was present in the cancer cell lines ZR75-30 and MDA-MB-435s, and tumors 21T, 95T, and 584T, but not 33T. The same blots were stripped and probed with ANKRD3 which demonstrated that the DNA was completely digested. PCDH8 methylation was seen in 6 of 21 (29%) breast tumor biopsies and 4 of 12 (33%) cell lines (Tables 6 and 7).

TABLE 6
Summary of inactivation of PCDH8 in breast cancers

| Breast tumors | Tumors | | Cell lines | | Total | |
|---|---|---|---|---|---|---|
| Reduced message | 13/41 | (31.7%) | 8/44 | (18.2%) | 21/85 | (24.7%) |
| Reduced protein | | | | | | |
| Biopsies | 8/35 | (22.9%) | N/A | | 8/35 | (22.9%) |
| TMA | 26/64 | (40.6%) | N/A | | 26/64 | (40.6%) |
| DCIS | 3/10 | (30.0%) | N/A | | 3/10 | (30.0%) |
| Somatic mutations | 2/116 | (1.7%) | 2/21 | (9.5%) | 4/137 | (2.9%) |
| Methylation | 6/21 | (28.6%) | 4/12 | (33.3%) | 10/33 | (30.3%) |

TMA = tissue microarray,
DCIS = ductal carcinoma in situ

TABLE 7
Methylation of PCDH8 correlates with loss of message.

| Cell lines | Meth/Unmeth | RT-PCR |
|---|---|---|
| MDA-MB-435s | M | − |
| ZR-75-30 | M | − |
| SUM229PE | M | − |
| SUM190PT | M | − |
| BT-483 | U | − |
| MDA-MB-436 | U | + |
| Hs578t | U | + |
| CAMA-1 | U | + |
| MCF7 | U | + |
| HCC1428 | U | + |
| HCC1937 | U | + |
| SUM159PT | U | + |

4/5 (80%) of cell lines with undetectable levels, of message exhibit methylation of the PCDH8 promoter.
M = methylated,
U = unmethylated,
− = no detection of message,
+ = detection of message.

Methylation in each case correlated with reduction of PCDH8 expression (FIGS. 12C, 13C and 13F). In patient biopsies, PCDH8 protein was expressed in the cytoplasm and on the membrane of the luminal and basal epithelial layers of normal breast ducts and lobules but was markedly reduced in tumors with a methylated promoter (n=6, p=0.0456) (FIGS. 13C and 13F, Table 8).

TABLE 8
PCDH8 loss correlates with loss of heterozygosity(LOH),
promoter methylation, loss of estrogen receptor (ER)
expression, loss of progesterone receptor (PR) expression,
and increased S-phase tumors.

| | PCDH8+ | PCDH8− |
|---|---|---|
| LOH+ | 10 | 7 |
| LOH− | 17 | 1 |
| | (p = 0.0178) | |
| Unmeth | 8 | 7 |
| Meth | 0 | 6 |
| | (p = 0.0456) | |
| ER+ | 26 | 2 |
| ER− | 3 | 5 |
| | (p = 0.001) | |
| PR+ | 21 | 0 |
| PR− | 8 | 7 |
| | (p = 0.001) | |

S-phase

| | | |
|---|---|---|
| >10 | 10 | 7 |
| <10 | 13 | 1 |
| | (p = 0.0454) | |

LOH+ = presence of LOH,
LOH− = absence of LOH,
Unmeth = unmethylated,
Meth = methylated.

To study the relationship between promoter methylation and PCDH8 silencing, MDA-MB-435s cells were treated with a DNA methyltransferase inhibitor, 5-aza-deoxycytidine. Treatment restored expression of PCDH8, showing that chromatin modification of the CpG island is involved in gene silencing in tumors (FIG. 13E).

### Example 11

### Loss of Heterozygosity and PCDH8 Mutations

Thirty-five of the breast tumors that had been evaluated for LOH and PCDH8 mutation were screened for loss of protein expression. Levels of PCDH8 protein were reduced relative to adjacent normal ducts and lobules in 8/35 (23%) tumors (FIG. 13G; Table 6). Downregulation of PCDH8 correlated with loss of heterozygosity (LOH) for 13q14 (p=0.0178), suggesting that 2 hits are required for PCDH8 inactivation (Table 8). In addition, reduced PCDH8 correlated with reduced estrogen receptor (p=0.001) and progesterone receptor (p=0.001), and increased S-phase tumors (p=0.0454), raising the possibility that PCDH8 may regulate cell proliferation (Table 8). Cases with missense mutations showed no evidence of reduced PCDH8 expression by staining. This observation suggests that the mutants are stable. Thus, these data support a model of tumor formation in which PCDH8 is commonly inactivated by a combination of LOH of one allele and promoter silencing or missense mutation of the remaining allele. In a separate series of breast tumors on a tissue microarray, reduced PCDH8 expression was found in tumor cells in 26/64 ductal breast cancers (41%) and 3/10 ductal carcinomas in situ (30%) (Table 6).

### Example 12

### Effect of PCDH8 Mutation on Cell Growth Migration Proliferation and Differentiation

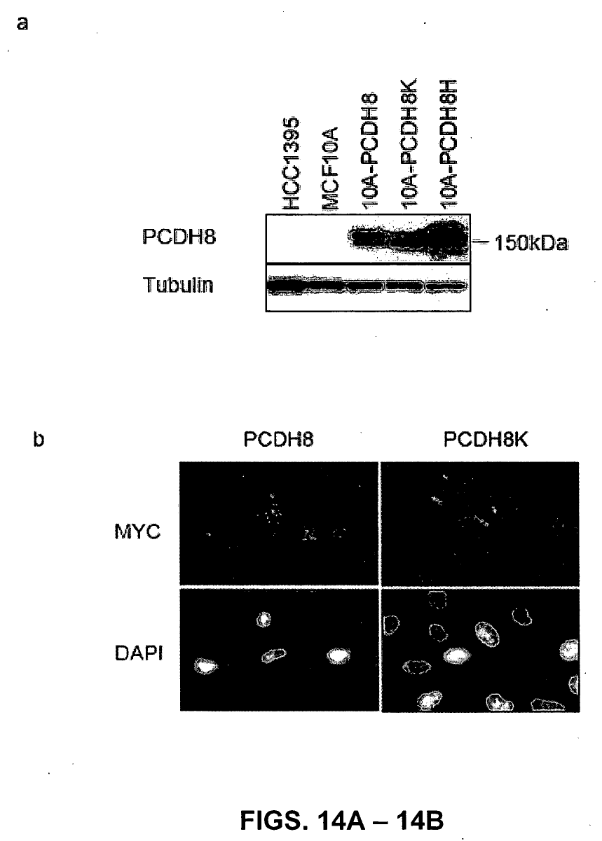

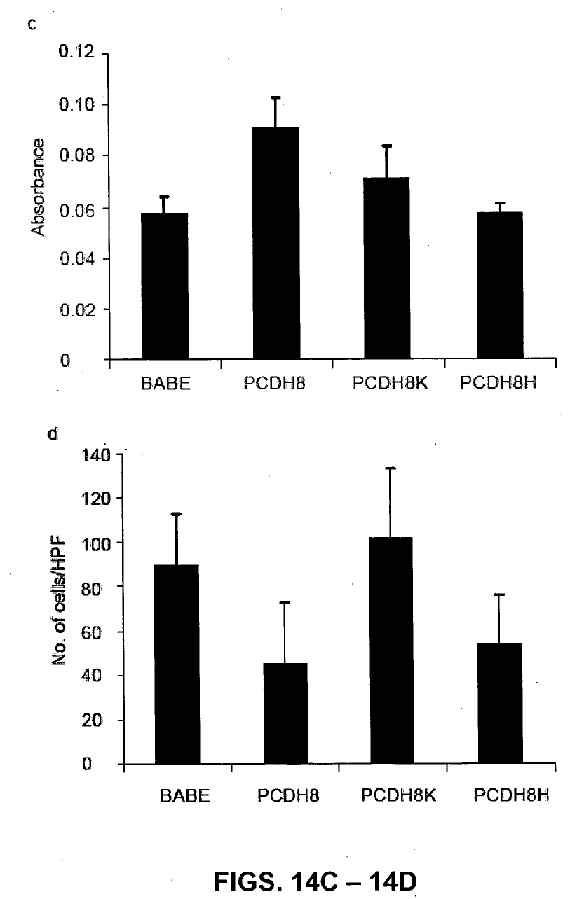

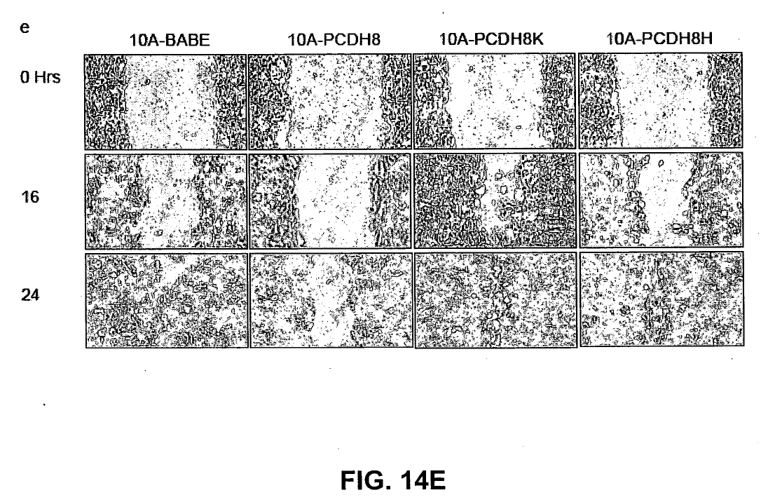

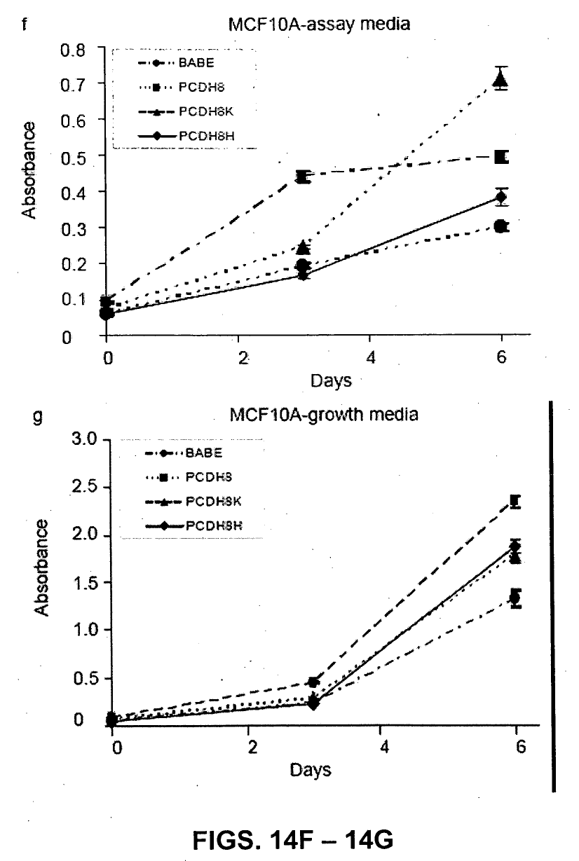

Having established that PCDH8 is a candidate tumor suppressor, studies were carried out to address whether introducing somatic mutations of PCDH8 into normal cells could alter cell growth (FIG. 14A). MCF10A, which expresses endogenous PCDH8, was used as a model of transformation (FIG. 12A). When grown in Matrigel, a single MCF10A cell can develop into a multicellular acinus, recapitulating the development of normal breast ducts (Debnath, J., Muthuswamy, S. K. & Brugge, J. S. Morphogenesis and oncogenesis of MCF-10A mammary epithelial acini grown in three-dimensional basement membrane cultures. Methods 30, 256-68 (2003)). Perturbation of mitogenic pathways by overexpression of ErbB2/HER2/Neu, for example, produces disorganized structures resembling breast cancers (Muthuswamy, S. K., Li, D., Lelievre, S., Bissell, M. J. & Brugge, J. S. ErbB2, but not ErbB1, reinitiates proliferation and induces luminal repopulation in epithelial acini. Nat Cell Biol 3, 785-92 (2001); Debnath, J., Walker, S. J. & Brugge, J. S. Akt activation disrupts mammary acinar architecture and enhances proliferation in an mTOR-dependent manner. J Cell Biol 163, 315-26 (2003)).

To explore the function of PCDH8 in mammary cell growth and differentiation MCF10A cells were infected with retroviruses expressing epitope-tagged wild-type PCDH8, the somatic mutants E146K (PCDH8K) and R343H (PCDH8H), or the empty vector pBABEpuro. The expression of exogenous PCDH8 was readily detectable and appeared to be higher than the endogenous level expressed in MCF10A (FIG. 14A). Immunostaining revealed that wild-type PCDH8 but not the E146K mutant was concentrated in delicate connections between cells (FIG. 14B).

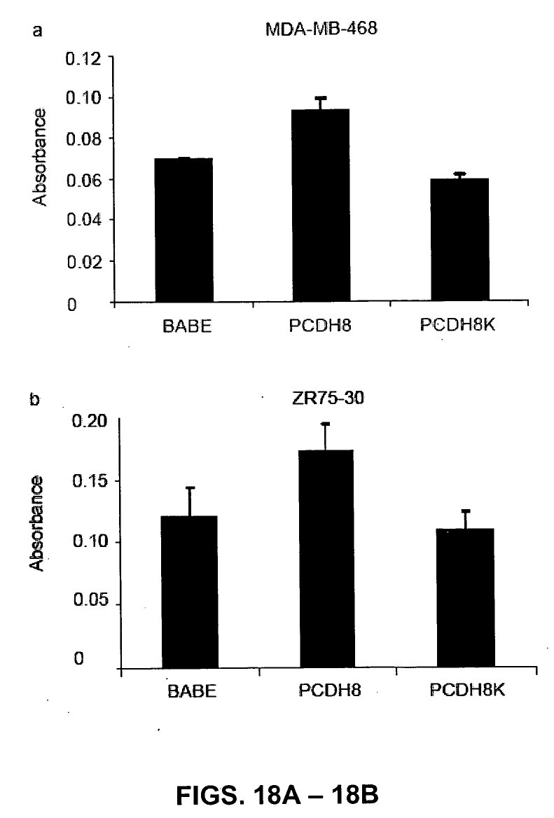

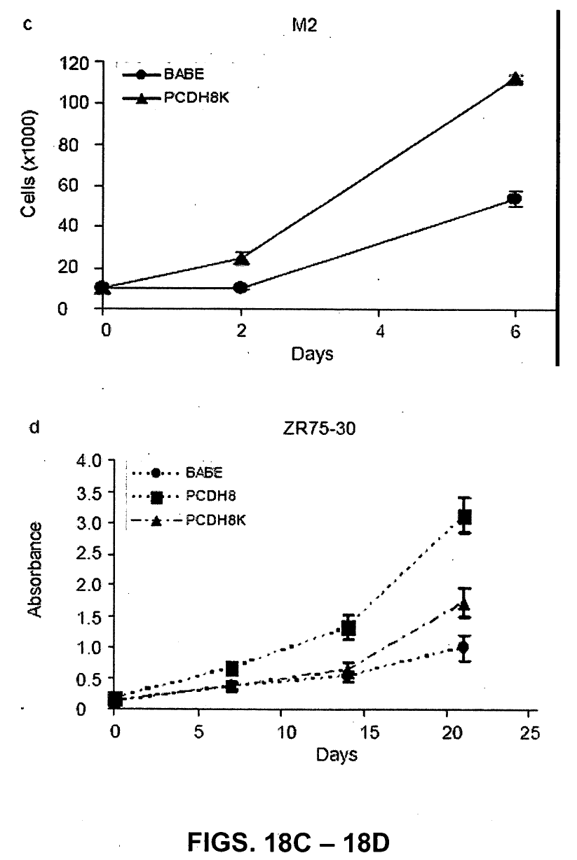

Considering that PAPC regulates embryonic cell movements and PCDH8 is expressed in membranes, studies were carried out to determine whether PCDH8 could alter adhesion and migration. While overexpression of PCDH8 improved plating efficiency, expression of PCDH8K or PCDH8H did not (FIG. 14C). Restoration of PCDH8 expression in the breast cancer cell lines MDA-MB-468 and ZR75-30 similarly improved plating efficiency (FIGS. 18A and 18B).

Studies were then carried out to determine whether PCDH8 could alter cell migration. In transwell migration assays, both wild type PCDH8 and mutant PCDH8H repressed migration (FIG. 14D). PCDH8K, on the other hand, was not measurably different from the empty vector. In wound healing assays, cells overexpressing PCDH8 showed reduced ability to migrate into the wound relative to empty vector control cells (FIG. 14E). In this context, both PCDH8K and PCDH8H mutants behaved like empty vector and closed the wound completely within 24 hours. These observations show that PCDH8 diminishes migration and that the mutations have different levels of impairment.

Studies were next carried out to determine whether mutant PCDH8 could affect proliferation and differentiation. Since wild-type MCF10A cells require exogenous EGF to proliferate, studies were carried out to determine whether PCDH8 tumor mutants could supplant the requirement for EGF. While vector control, wild-type PCDH8, and PCDH8H cells were growth limited in the absence of EGF, PCDH8K cells were capable of modest proliferation (FIG. 14F). Similarly, in another immortalized breast line dependent on EGF signaling, M2, cells expressing PCDH8K had enhanced proliferation relative to empty vector control cells in the absence of exogenous EGF (FIG. 18C).

PCDH8-mediated proliferation was context dependent. In both MCF10A and ZR75-30, expression of PCDH8 increased proliferation in EGF-containing and serum-rich media compared to mutant PCDH8 or vector control (FIGS. 14G and 18D). Thus, the proliferative potential conferred by wild type or mutant PCDH8 is regulated by extracellular stimuli.

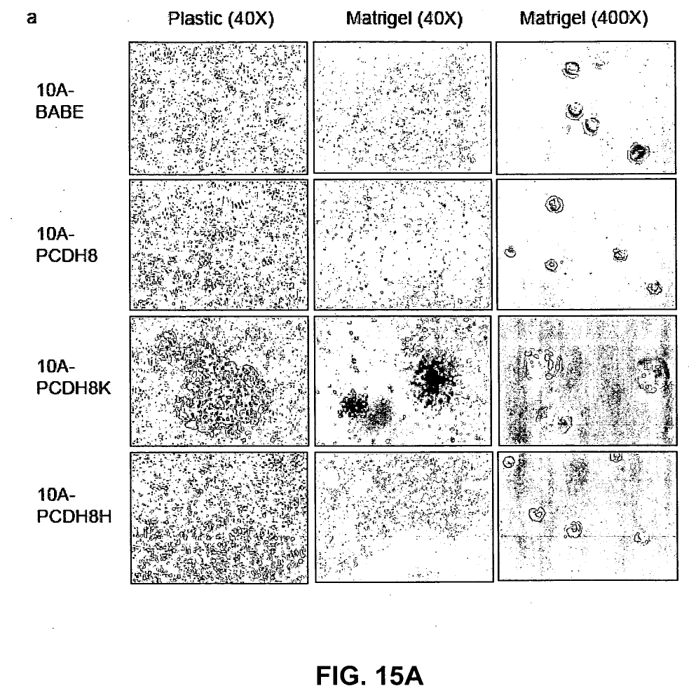

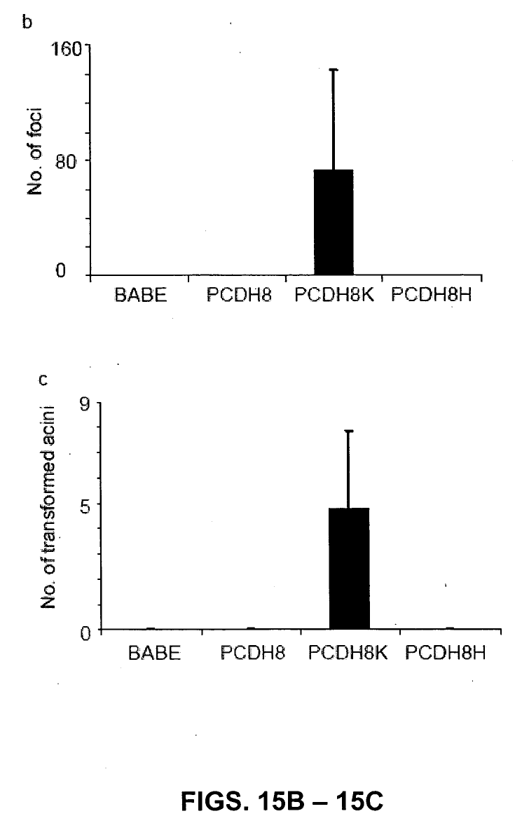

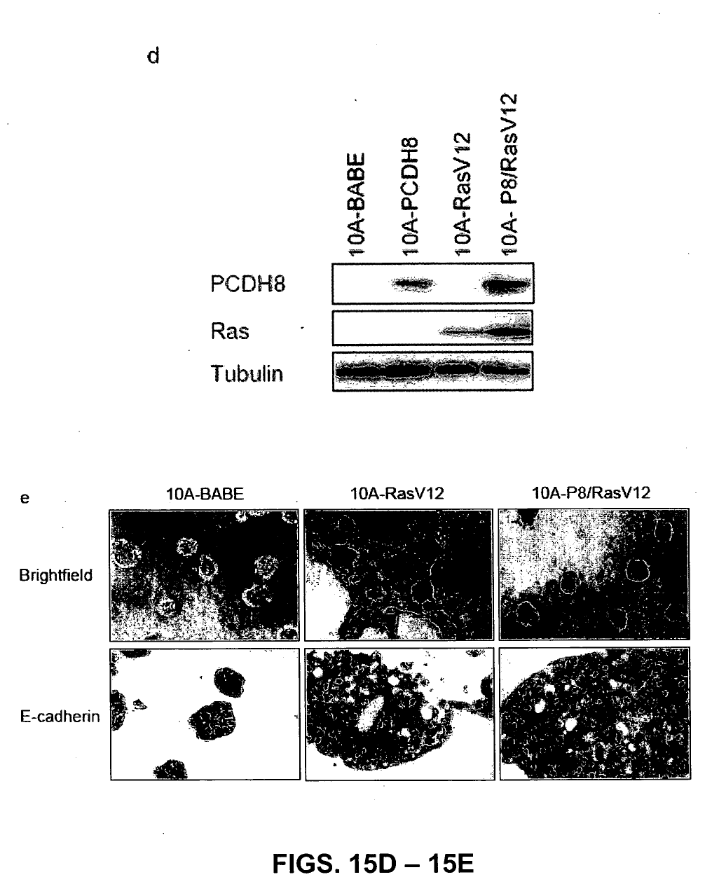

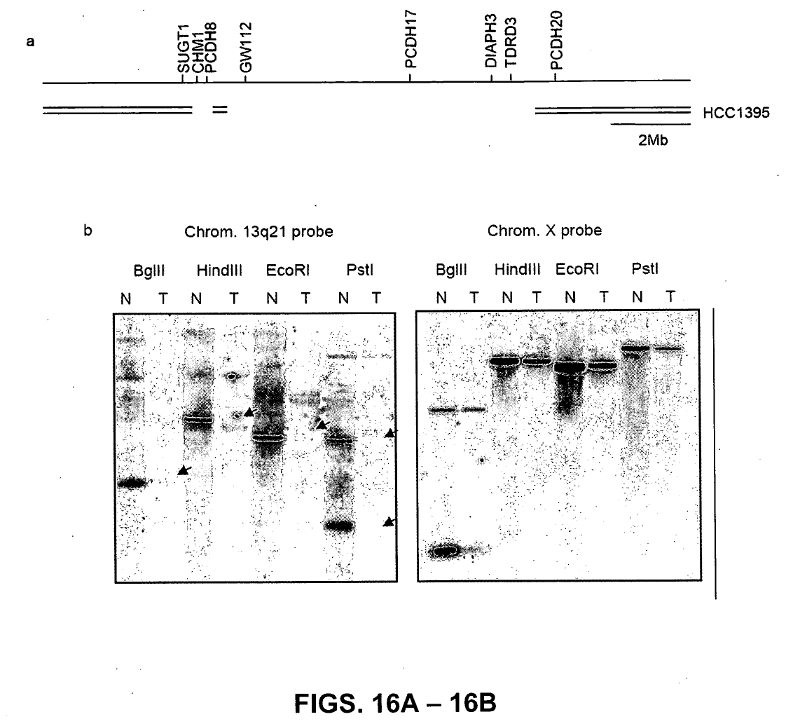

On the other hand, under both enriched and limited growth factor conditions, PCDH8K conferred increased susceptibility to transformation. A subset of 10A-PCDH8K cells was able to form foci when grown on plastic, whereas control, wt PCDH8, and PCDH8H cells were not (FIGS. 15A and 15B). When grown in reduced growth factor Matrigel, wt PCDH8, mutant PCDH8H, and empty vector cells developed into small spheroids (FIG. 15A). In contrast, PCDH8K cells developed into large acini (FIG. 15A). A subset of these cells transformed into complex, spiculated organoids, at an average incidence of 5:5000 acini (FIGS. 15A and 15C). Thus, mutation of PCDH8 not only facilitates proliferation in an EGF-independent manner but also promotes cellular transformation. This is consistent with observations that loss of PCDH8 in breast tumors correlated with increased S-phase.

Given the phenotype of PCDH8K in Matrigel, studies were carried out to determine whether wild type PCDH8 could perturb acinus development and morphogenesis in transformed cells. MCF10A cells expressing RasV12 were used as a model because RasV12 promotes loss of E-cadherin expression (FIGS. 15D and 15E). When cultured in Matrigel, these cells grow into spheroid structures interconnected by a weblike pattern of migrating cells (FIG. 15E).

Overexpression of wild type PCDH8 was able to inhibit the migration, consistent with its adhesive and anti-motility properties (FIG. 15E). Moreover, this inhibition was independent of E-cadherin expression (FIG. 15E).

### Example 13

### PCDH8 Cloning and Mutagenesis

A human PCDH8 clone was purchased from Origene, clone ID FB1851_H03, pCMV6-XL4-PCDH8. This clone contained a missense change. The wild type sequence was created using QuikChange XL Site-Directed Mutagenesis Kit (Stratagene), primers CAGGACACCTACGAGCTGGACGTGCG (SEQ ID NO:67) and CGCACGTCCAGCTCGTAGGTGTCCTG (SEQ ID NO:68). pBABE-PCDH8-myc was generated by PCR amplification and cloned into pBABEpuro between EcoRI and SalI sites. PCDH8-E146K mutant was generated using primers

| | |
|---|---|
| GGTAGAAGGTGTCCAAGGGTGCGGCAGTG | (SEQ ID NO: 69) |
| and | |
| CACTGCCGCACCCTTGGACACCTCTACC; | (SEQ ID NO: 70) |
| PCDH8-R343H mutant with primers | |
| GCAAGGTCATCGTGCACATCCGAGACGTC | (SEQ ID NO: 71) |
| and | |
| ATTGACGTCTCGGATGTGCACGATGACCT. | (SEQ ID NO: 72) |

### Example 14

### Retrovirus Production and Infection

Phoenix-ampho cells for retrovirus production were used. A T75 flask of cells was transfected with 21 ug plasmid using Lipofectamine 2000 (Invitrogen). Virus was harvested 48-72 hours post-transfection, stabilized with FBS, and passed through a 0.45 um filter. Cells were infected with viruses in the presence of 8 μg/ml polybrene and selected with 1 μg/ml puromycin.

### Example 15

### Immunofluorescence

MCF10A cells expressing MYC epitope-tagged proteins were plated onto sterile cover slips in a 6-well dish. Sixteen hours after plating, cells were fixed in 2% paraformaldehyde in PBS pH 7.4 for 30 minutes at room temperature. Cells were washed for 20 minutes in PBS, permeabilized for 1 hour in buffer A (5% goat serum, 0.1% Triton X-100 in PBS), and incubated with 1:1000 dilution of mouse monoclonal anti-MYC (9E10) antibody (Santa Cruz) in buffer A. Cells were washed in PBS, and incubated with 1:600 dilution of Alexafluor 568 anti-mouse antibody (Molecular Probes) and counterstained with DAPI (0.15 μg/ml in water).

### Example 16

### Western Blotting and Immunohistochemistry

Whole cell lysates were used in all Western blots. Paraffin sections were stained with 1:5000 dilution of the anti-PCDH8 antibody. Slides were developed with ABC-DAB (Vector, Biogenics). Antibodies: Anti-PCDH8 was raised against amino acids 1052-1070 (YQSPPGRYLSPKKGANENV (SEQ ID NO:73)) in rabbits and affinity purified (NCBI accession number AAC70009). Other antibodies were commercially available: anti-tubulin (Tu27)(Covance), anti-vinculin (hVIN-1)(Sigma), anti-Myc (9E10)(Santa Cruz), anti-E-cadherin (BD Biosciences), anti-v-H-ras (Ab-1)(Calbiochem).

### Example 17

### Morphogenesis Assay

40 μl growth-factor reduced Matrigel (BD Biosciences) was plated on 8-chamber slides (Costar). MCF10A cell lines were grown in each chamber as described (Debnath, J., Muthuswamy, S. K. & Brugge, J. S. Morphogenesis and oncogenesis of MCF-10A mammary epithelial acini grown in three-dimensional basement membrane cultures. Methods 30, 256-68 (2003)).

### Example 18

### Adhesion and Proliferation Assays

MCF10A, MDA-MB-468 and ZR75-30:3,000 cells/well were plated on 48 well plates in growth media. For MCF10A, media was changed to assay media (DMEM:F12, 0.5% horse serum, 0.5 ug/ml hydrocortisone, 100 ng/ml cholera toxin, 10 μg/ml insulin, 1× penicillin/streptomycin) 12-16 hrs after plating. At day of harvest (12-16 hrs after plating for adhesion assays), cells were washed in PBS, stained with crystal violet, and solubilized with 10% acetic acid. Absorbance at 590 nm was read with a MicroQuant universal microplate spectrophotometer plate reader (BIO-TEK). For M2 assays: 100,000 cells were plated into 6 well plates. 12-16 hrs after plating, cells were grown in DFCI-1 media, without supplemental EGF. Band, V. & Sager, R. Distinctive traits of normal and tumor-derived human mammary epithelial cells expressed in a medium that supports long-term growth of both cell types (Proc Natl Acad Sci USA 86, 1249-53 (1989)). At day of harvest, cells were trypsinized and counted. All assays were performed in triplicate. Error bars of all adhesion assays and M2 proliferation assays represent standard deviations. Error bars of MCF10A and ZR75-30 proliferation assays represent standard errors of the mean.

### Example 19

### Migration Assays

Transwell migration assay: 50,000 cells were plated in the upper chambers of 8 micron transwell cell culture plates (Costar). Lower chambers contained either assay media (without chemoattractant) or growth media (with chemoattractant). After 16-24 hrs of incubation, cells on the upper surface of the filters were removed with swabs. Migrating cells were stained with crystal violet. For each filter, 20 high power fields were counted. Wound healing assay: Equal numbers of cells were plated on a 6-well plate. A single wound was introduced using a P20 pipette tip and media was replaced.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for determining whether a subject has neoplasia, comprising assaying a diagnostic sample of the subject for expression of at least one protocadherin selected from the group consisting of PCDH8, PCDH9, and PCDH17, wherein detection of PCDH8 expression decreased below normal is diagnostic of neoplasia in the subject, detection of PCDH9 expression decreased below normal is diagnostic of neoplasia in the subject, and detection of PCDH17 expression elevated above normal is diagnostic of neoplasia in the subject.

2. The method of claim 1, wherein the neoplasia is a carcinoma, a lymphocytic leukemia, a myeloid leukemia, a malignant lymphoma, a malignant melanoma, a myeloproliferative disease, a sarcoma, or a mixed type of neoplasia.

3. The method of claim 2, wherein the neoplasia is breast cancer or prostate cancer.

4. The method of claim 1, wherein the diagnostic sample is assayed with at least one agent reactive with PCDH8, PCDH9, and/or PCDH17.

5. The method of claim 4, wherein the at least one agent is labeled with a detectable marker.

6. The method of claim 4, wherein the at least one agent is an antibody.

7. The method of claim 1, wherein the diagnostic sample is assayed using at least one nucleic acid probe that hybridizes to nucleic acid encoding PCDH8, PCDH9, and/or PCDH17.

8. The method of claim 7, wherein the nucleic acid probe is DNA or RNA.

9. A method for assessing the efficacy of therapy to treat neoplasia in a subject who has undergone or is undergoing treatment for neoplasia, comprising assaying a diagnostic sample of the subject for expression of at least one protocadherin selected from the group consisting of PCDH8, PCDH9, and PCDH17, wherein normal expression of PCDH8, PCDH9, and PCDH17 in the diagnostic sample is indicative of successful therapy to treat neoplasia, and wherein PCDH8 expression decreased below normal in the diagnostic sample and/or PCDH9 expression decreased below normal in the diagnostic sample and/or PCDH7 expression elevated above normal in the diagnostic sample is indicative of a need to continue therapy to treat neoplasia.

10. A method for assessing the prognosis of a subject who has neoplasia, comprising assaying a diagnostic sample of the subject for expression of at least one protocadherin selected from the group consisting of PCDH8, PCDH9, and PCDH17, wherein the subject's prognosis improves with an increase in expression of PCDH8 in the diagnostic sample and/or an increase in expression of PCDH9 in the diagnostic sample and/or a decrease in expression of PCDH17 in the diagnostic sample, and wherein the subject's prognosis worsens with a decrease in expression of PCDH8 in the diagnostic sample and/or a decrease in expression of PCDH9 in the diagnostic sample and/or an increase in expression of PCDH17 in the diagnostic sample.

11. A method for determining whether a subject has neoplasia, comprising assaying a diagnostic sample of the subject for methylation of PCDH8 promoter and/or methylation of PCDH9 promoter, wherein detection of methylation of the PCDH8 promoter and/or the PCDH9 promoter is diagnostic of neoplasia in the subject.

12. The method of claim 11, wherein the neoplasia is breast cancer or prostate cancer.

13. A method for assessing the efficacy of therapy to treat neoplasia in a subject who has undergone or is undergoing treatment for neoplasia, comprising assaying a diagnostic sample of the subject for methylation of PCDH8 promoter and/or methylation of the PCDH9 promoter, wherein no detection of methylation of the PCDH8 promoter and/or the PCDH9 promoter in the diagnostic sample is indicative of successful therapy to treat neoplasia, and wherein detection of methylation of the PCDH8 promoter and/or the PCDH9 promoter in the diagnostic sample is indicative of a need to continue therapy to treat neoplasia.

14. A method for assessing the prognosis of a subject who has neoplasia, comprising assaying a diagnostic sample of the subject for methylation of PCDH8 promoter and/or PCDH9 promoter, wherein the subject's prognosis improves with a decrease in methylation of the PCDH8 promoter and/or the PCDH9 promoter in the diagnostic sample, and wherein the subject's prognosis worsens with an increase in methylation of the PCDH8 promoter and/or the PCDH9 promoter in the diagnostic sample.

15. A method for determining whether a subject has neoplasia, comprising assaying a diagnostic sample of the subject for at least one mutation selected from the group consisting of a PCDH8 mutation and a PCDH17 mutation.

16. The method of claim 15, wherein the PCDH8 mutation and/or the PCDH17 mutation results in a loss of heterozygosity in chromosome 13.

17. The method of claim 15, wherein the PCDH8 mutation is selected from the group consisting of a G436A mutation, a G1028A mutation, a G748A mutation, a C-IT mutation, a A1099G mutation, and a T2015A mutation.

18. The method of claim 15, wherein the PCDH8 mutation is selected from the group consisting of an E146K mutation, an R343H mutation, a V250M mutation, a T367A mutation, and an L672Q mutation.

19. The method of claim 15, wherein the PCDH17 mutation is selected from the group consisting of a deletion in exon 1, a C1364T mutation, and a G2899T mutation.

20. The method of claim 15, wherein the PCDH17 mutation is selected from the group consisting of a P222S mutation and an E733D mutation.

21. A kit for use in detecting neoplasia, comprising:
(a) at least one agent reactive with PCDH8, PCDH9, and/or PCDH17; and
(b) reagents suitable for detecting expression of at least one protocadherin selected from the group consisting of PCDH8, PCDH9, and PCDH17.

22. The kit of claim 21, wherein the at least one agent is labeled with a detectable marker.

23. The kit of claim 21, wherein the at least one agent is an antibody.

24. A kit for use in detecting neoplasia, comprising:
(a) at least one nucleic acid probe that hybridizes to nucleic acid encoding PCDH8, PCDH9, and/or PCDH17; and
(b) reagents suitable for detecting expression of at least one nucleic acid encoding at least one protocadherin selected from the group consisting of PCDH8, PCDH9, and PCDH17.

25. The kit of claim 24, wherein the at least one nucleic acid probe is labeled with a detectable marker.

26. A method for treating or preventing neoplasia in a subject, comprising modulating level and/or function of at least one protocadherin in the subject, wherein the at least one protocadherin is selected from the group consisting of PCDH8, PCDH9, and PCDH17.

27. The method of claim 26, wherein the neoplasia is a carcinoma, a lymphocytic leukemia, a myeloid leukemia, a malignant lymphoma, a malignant melanoma, a myeloproliferative disease, a sarcoma, or a mixed type of neoplasia.

28. The method of claim 27, wherein the neoplasia is breast cancer or prostate cancer.

29. The method of claim 26, wherein the level and/or function of the at least one protocadherin is modulated in the subject by administering to the subject at least one modulator of protocadherin expression selected from the group consisting of a modulator of PCDH8 expression, a modulator of PCDH9 expression, and a modulator of PCDH17 expression.

30. The method of claim 29, wherein the modulator is administered orally, intradermally, intramuscularly, intraperitoneally, intravenously, or subcutaneously.

31. The method of claim 26, wherein the modulation of level and/or function is selected from the group consisting of increasing level and/or function of PCDH8 in the subject, increasing level and/or function of PCDH9 in the subject, and decreasing level and/or function of PCDH17 in the subject.

32. The method of claim 31, wherein the level and/or function of PCDH8 is increased in the subject by administering to the subject an amount of PCDH8 protein or a modulator of PCDH8 expression.

33. The method of claim 31, wherein the level and/or function of PCDH9 is increased in the subject by administering to the subject an amount of PCDH9 protein or a modulator of PCDH9 expression.

34. The method of claim 31, wherein the level and/or function of PCDH8 is increased in the subject by administering to the subject a nucleic acid sequence encoding PCDH8, in a manner permitting expression of PCDH8 in the subject.

35. The method of claim 31, wherein the level and/or function of PCDH9 is increased in the subject by administering to the subject a nucleic acid sequence encoding PCDH9, in a manner permitting expression of PCDH9 in the subject.

36. The method of claim 31, wherein the level and/or function of PCDH8 or PCDH9 is increased in the subject by altering chromatin silencing or PCDH methylation in the subject.

37. The method of claim 36, wherein the level and/or function of PCDH8 or PCDH9 is increased in the subject by administering 5-aza-deoxycytidine and/or trichostatin A to the subject.

38. The method of claim 31, wherein the level and/or function of PCDH17 is decreased in the subject with antisense RNA or RNAi.

39. The method of claim 31, wherein the level and/or function of PCDH17 is decreased in the subject by administering to the subject an agent reactive with PCDH17.

40. The method of claim 39, wherein the agent is an antibody.

41. The method of claim 39, wherein the agent is co-administered with an additional therapeutic agent.

42. The method of claim 41 wherein the fusion protein is VEGF-Trap.

43. A pharmaceutical composition, comprising a modulator of PCDH8 expression or a PCDH8 protein, in an amount effective to treat or prevent neoplasia in a subject to whom the composition is administered, and a pharmaceutically-acceptable carrier.

44. A pharmaceutical composition, comprising a modulator of PCDH9 expression or a PCDH9 protein, in an amount effective to treat or prevent neoplasia in a subject to whom the composition is administered, and a pharmaceutically-acceptable carrier.

45. A pharmaceutical composition, comprising an agent reactive with PCDH17, in an amount effective to treat or prevent neoplasia in a subject to whom the composition is administered, and a pharmaceutically-acceptable carrier.

46. A method for identifying an agent for use in treating and/or preventing neoplasia, comprising the steps of:
(a) obtaining a collection of cells having at least one characteristic selected from the group consisting of PCDH8 expression decreased below normal, PCDH9 expression decreased below normal, and PCDH17 expression elevated above normal;
(b) contacting a candidate agent with the cells; and
(c) determining the effect, if any, of the candidate agent on PCDH8 expression, PCDH9 expression, and/or PCDH17 expression in the cells.

47. The method of claim 46, further comprising the steps of:
(d) contacting the candidate agent with neoplastic cells having at least one characteristic selected from the group consisting of PCDH8 expression decreased below normal, PCDH9 expression decreased below normal, and PCDH17 expression elevated above normal; and
(e) determining if the agent has an effect on proliferation of the neoplastic cells.

48. The agent identified by the method of claim 46.

49. The agent identified by the method of claim 47.

50. A method for treating or preventing neoplasia in a subject, comprising administering to the subject an amount of the agent of claim 48 effective to treat or prevent the neoplasia.

51. A method for identifying an agent for use in treating and/or preventing neoplasia, comprising the steps of:
(a) obtaining an animal comprising cells having at least one characteristic selected from the group consisting of PCDH8 expression decreased below normal, PCDH9 expression decreased belong normal, and PCDH17 expression elevated above normal;
(b) administering a candidate agent to the animal; and
(c) determining the effect, if any, of the candidate agent on PCDH8 expression, PCDH9 expression, and/or PCDH17 expression in the animal.

52. The method of claim 51, further comprising the steps of:
(d) contacting the candidate agent with an animal comprising neoplastic cells, wherein the neoplastic cells have at least one characteristic selected from the group consisting of PCDH8 expression decreased below normal, PCDH9 expression decreased below normal, and PCDH17 expression elevated above normal; and
(e) determining if the agent has an effect on proliferation of the neoplastic cells.

53. The agent identified by the method of claim 51.

54. The agent identified by the method of claim 52.

55. A method for treating or preventing neoplasia in a subject, comprising administering to the subject an amount of the agent of claim 53 effective to treat or prevent the neoplasia.


SEQUENCE LISTING (84 sequences)

SEQ ID NO: 1 - DNA, 22 bp, Artificial
  Synthetic Primer
ccttagcctttcggatcgcact

SEQ ID NO: 2 - DNA, 18 bp, Artificial
  Synthetic Primer
atgagtcctgtgaggcgt

SEQ ID NO: 3 - DNA, 18 bp, Artificial
  Synthetic Primer
ctcgcgtctgcagctcca

SEQ ID NO: 4 - DNA, 19 bp, Artificial
  Synthetic Primer
aaggccagcacgcactgcg

SEQ ID NO: 5 - DNA, 19 bp, Artificial
  Synthetic Primer
cgcagtgcgtgctggcctt

SEQ ID NO: 6 - DNA, 18 bp, Artificial
  Synthetic Primer
agcctttcggatcgcact

SEQ ID NO: 7 - DNA, 17 bp, Artificial
  Synthetic Primer
ctgtgcggctcggccag

SEQ ID NO: 8 - DNA, 20 bp, Artificial
  Synthetic Primer
tttcgcgtggagctgcagac

SEQ ID NO: 9 - DNA, 17 bp, Artificial
  Synthetic Primer
gacgcgagcggacggcg

SEQ ID NO: 10 - DNA, 20 bp, Artificial
  Synthetic Primer
ttgtcgttctcgtcgcccac

SEQ ID NO: 11 - DNA, 16 bp, Artificial
  Synthetic Primer
cgcgatcaggccgcct

SEQ ID NO: 12 - DNA, 18 bp, Artificial
  Synthetic Primer
tgccacttcgctggtgcc

SEQ ID NO: 13 - DNA, 19 bp, Artificial
  Synthetic Primer
gtgcgctgcgccctctatg

SEQ ID NO: 14 - DNA, 18 bp, Artificial
  Synthetic Primer
gccctctatgggcacgag

SEQ ID NO: 15 - DNA, 20 bp, Artificial
  Synthetic Primer
ctgttaccacgaagctgaca

SEQ ID NO: 16 - DNA, 18 bp, Artificial
  Synthetic Primer
ccggtctatgaggtgtcg

SEQ ID NO: 17 - DNA, 20 bp, Artificial
  Synthetic Primer
ctggaccagaacgaccatgc

SEQ ID NO: 18 - DNA, 17 bp, Artificial
  Synthetic Primer
acagttgcggtggtggt

SEQ ID NO: 19 - DNA, 16 bp, Artificial
  Synthetic Primer
caggagccgcgcgaag

SEQ ID NO: 20 - DNA, 16 bp, Artificial
  Synthetic Primer
caggagccgcgcgaag

SEQ ID NO: 21 - DNA, 18 bp, Artificial
  Synthetic Primer
ctgcagcagggaataggg

SEQ ID NO: 22 - DNA, 20 bp, Artificial
  Synthetic Primer
gaagccttcgccatcggccg

SEQ ID NO: 23 - DNA, 18 bp, Artificial
  Synthetic Primer
ctgcagcagggaataggg

SEQ ID NO: 24 - DNA, 19 bp, Artificial
  Synthetic Primer
cgggagccgaggctccgcc

SEQ ID NO: 25 - DNA, 24 bp, Artificial
  Synthetic Primer
ggtagggtgggtgcattcaggtag

SEQ ID NO: 26 - DNA, 20 bp, Artificial
  Synthetic Primer
gtagacaacctgtcgcactt

SEQ ID NO: 27 - DNA, 24 bp, Artificial
  Synthetic Primer
gcgggaaagaagagtcctcaccac

SEQ ID NO: 28 - DNA, 20 bp, Artificial
  Synthetic Primer
ctcaccactctgcatgtggt

SEQ ID NO: 29 - DNA, 24 bp, Artificial
  Synthetic Primer
ctgacccaggctgacatgctgaag

SEQ ID NO: 30 - DNA, 20 bp, Artificial
  Synthetic Primer
ggaagcctgaatatgtataa

SEQ ID NO: 31 - DNA, 24 bp, Artificial
  Synthetic Preimer
ttaggggcttctcggagtgacctg

SEQ ID NO: 32 - DNA, 20 bp, Artificial
  Synthetic Primer
cggagtgacctgtatatgtg

SEQ ID NO: 33 - DNA, 20 bp, Artificial
  Synthetic Primer
tggctcctccagtccgattg

SEQ ID NO: 34 - DNA, 20 bp, Artificial
  Synthetic Primer
ctcctgcccccaccttacag

SEQ ID NO: 35 - DNA, 20 bp, Artificial
  Synthetic Primer
taggtgcggagcccattctc

SEQ ID NO: 36 - DNA, 20 bp, Artificial
  Synthetic Primer
ggcgtcggggtcatgtgcgc

SEQ ID NO: 37 - DNA, 22 bp, Artificial
  Synthetic Primer
atccaggacatcaacgacaacg

SEQ ID NO: 38 - DNA, 20 bp, Artificial
  Synthetic Primer
cgcgccctccttctcctcgg

SEQ ID NO: 39 - DNA, 20 bp, Artificial
  Synthetic Primer
cccttcacacggattaggcc

SEQ ID NO: 40 - DNA, 18 bp, Artificial
  Synthetic Primer
cggtcttggggtcgatgg

SEQ ID NO: 41 - DNA, 25 bp, Artificial
  Synthetic Primer
ccatcgaccccaagaccggcctaat

SEQ ID NO: 42 - DNA, 20 bp, Artificial
  Synthetic Primer
tgaagggcaatctggactat

SEQ ID NO: 43 - DNA, 25 bp, Artificial
  Synthetic Primer
tagagccctttggtgaaccgaggcg

SEQ ID NO: 44 - DNA, 21 bp, Artificial
  Synthetic Primer
ttgtcgttctctgtctagaat

SEQ ID NO: 45 - DNA, 19 bp, Artificial
  Synthetic Primer
ccagtcgttcgcgatcaag

SEQ ID NO: 46 - DNA, 18 bp, Artificial
  Synthetic Primer
attctagacgagaacgac

SEQ ID NO: 47 - DNA, 22 bp, Artificial
  Synthetic Primer
gtcgatctcaaacaggtggtcg

SEQ ID NO: 48 - DNA, 20 bp, Artificial
  Synthetic Primer
gttgccgtccacgatctcgt

SEQ ID NO: 49 - DNA, 21 bp, Artificial
  Synthetic Primer
cgtctcacctacgagatcgtg

SEQ ID NO: 50 - DNA, 20 bp, Artificial
  Synthetic Primer
ggacggcaacgacgaccacc

SEQ ID NO: 51 - DNA, 20 bp, Artificial
  Synthetic Primer
ggctgctcaccacgttcatg

SEQ ID NO: 52 - DNA, 19 bp, Artificial
  Synthetic Primer
gttcatggcgttcctctcc

SEQ ID NO: 53 - DNA, 25 bp, Artificial
  Synthetic Primer
agggcaagaagaagaagatcaacaa

SEQ ID NO: 54 - DNA, 20 bp, Artificial
  Synthetic Primer
atgatatcatgctggtgcag

SEQ ID NO: 55 - DNA, 25 bp, Artificial
  Synthetic Primer
catttttttctgctctgggaattcc

SEQ ID NO: 56 - DNA, 19 bp, Artificial
  Synthetic Primer
caaaggtttcaggacagct

SEQ ID NO: 57 - DNA, 25 bp, Artificial
  Synthetic Primer
tccactaggaaatcttgttgctctg

SEQ ID NO: 58 - DNA, 20 bp, Artificial
  Synthetic Primer
atttgtccagtgtacaacta

SEQ ID NO: 59 - DNA, 25 bp, Artificial
  Synthetic Primer
ccattccactgtgataaactgcaac

SEQ ID NO: 60 - DNA, 20 bp, Artificial
  Synthetic Primer
actgcttaagctttacactt

SEQ ID NO: 61 - DNA, 18 bp, Artificial
  Synthetic Primer
ggatttgctgttttctct

SEQ ID NO: 62 - DNA, 18 bp, Artificial
  Synthetic Primer
gttttgcagaaccagaag

SEQ ID NO: 63 - DNA, 18 bp, Artificial
  Synthetic Primer
accaggagggatcatttt

SEQ ID NO: 64 - DNA, 20 bp, Artificial
  Synthetic Primer
caagaaaatgccaatgcctt

SEQ ID NO: 65 - DNA, 19 bp, Artificial
  Synthetic Primer
gtgcatcgagccttgcacc

SEQ ID NO: 66 - DNA, 20 bp, Artificial
  Synthetic Primer
agccagggctcctggttttg

SEQ ID NO: 67 - DNA, 26 bp, Artificial
  Synthetic Primer
caggacacctacgagctggacgtgcg

SEQ ID NO: 68 - DNA, 26 bp, Artificial
  Synthetic Primer
cgcacgtccagctcgtaggtgtcctg

SEQ ID NO: 69 - DNA, 29 bp, Artificial
  Synthetic Primer
ggtagaaggtgtccaagggtgcggcagtg

SEQ ID NO: 70 - DNA, 28 bp, Artificial
  Synthetic Primer
cactgccgcacccttggacacctctacc

SEQ ID NO: 71 - DNA, 29 bp, Artificial
  Synthetic Primer
gcaaggtcatcgtgcacatccgagacgtc

SEQ ID NO: 72 - DNA, 29 bp, Artificial
  Synthetic Primer
attgacgtctcggatgtgcacgatgacct

SEQ ID NO: 73 - protein, 19 aa, Artificial
  Synthetic peptide
YQSPPGRYLSPKKGANENV

SEQ ID NO: 74 - DNA, 22 bp, Artificial
  Synthetic Primer
atctccctgatcaagactcaat

SEQ ID NO: 75 - DNA, 21 bp, Artificial
  Synthetic Primer
actgtgagaaagtggaaagta

SEQ ID NO: 76 - DNA, 19 bp, Artificial
  Synthetic Primer
tggcggtgtggaaaggaca

SEQ ID NO: 77 - DNA, 20 bp, Artificial
  Synthetic Primer
cggagtgacctgtatatgtg

SEQ ID NO: 78 - DNA, 20 bp, Artificial
  Synthetic Primer
aggcttttgagttcaaggtg

SEQ ID NO: 79 - DNA, 19 bp, Artificial
  Synthetic Primer
gtaagtctcagtctcaaca

SEQ ID NO: 80 - DNA, 20 bp, Artificial
  Synthetic Primer
agaggctattccaggcaccg

SEQ ID NO: 81 - DNA, 21 bp, Artificial
  Synthetic Primer
ctctcggaatcacgctctttg

SEQ ID NO: 82 - DNA, 20 bp, Artificial
  SYnthetic Primer
ggacgacctacggaagtgac

SEQ ID NO: 83 - DNA, 20 bp, Artificial
  Synthetic Primer
ctaactccactcacaaagcc

SEQ ID NO: 84 - protein, 1070 aa, Homo sapiens
MSPVRRWGSPCLFPLQLFSLCWVLSVAQSKTVRYSTFEEDAPGTVIGTLAEDLHMKVSGDTSFRLMKQFNSSLLRVREGDGQLTVGDAGLDRERLCGQAPQCVLAFDVVSFSQEQFRLVHVEVEVRDVNDHAPRFPRAQIPVEVSEGAAVGTRIPLEVPVDEDVGANGLQTVRLAEPHSPFRVELQTRADGAQCADLVLLQELDRESQAAYSLELVAQDGGRPPRSATAALSVRVLDANDHSPAFPQGAVAEVELAEDAPVGSLLLDLDAADPDEGPNGDVVFAFGARTPPEARRLFRLDPRSGRLTLAGPVDYERQDTYELDVRAQDRGPGPRAATCKVIVRIRDVNDNAPDIAITPLAAPGAPATSPFAAAAAAAALGGADASSPAGAGTPEAGATSLVPEGAARESLVALVSTSDRDSGANGQVRCALYGHEHFRLQPAYAGSYLVVTAASLDRERIAEYNLTLVAEDRGAPPLRTVRPYTVRVGDENDNAPLFTRPVYEVSVRENNPPGAYLATVAARDRDLGRNGQVTYRLLEAEVGRAGGAVSTYVSVDPATGAIYALRSFDYETLRQLDVRIQASDGGSPQLSSSALVQVRVLDQNDHAPVLVHPAPANGSLEVAVPGRTAKDTVVARVQARDADEGANGELAFELQQQEPREAFAIGRRTGEILLTGDLSQEPPGRVFRALLVISDGGRPPLTTTATVSFVVTAGGGRGPAAPASAGSPERSRPPGSRLGVSGSVLQWDTPLIVIIVLAGSCTLLLAAIIAIATTCNRRKKEVRKGGALREERPGAAGGGASAPGSPEEAARGAGPRPNMFDVLTFPGTGKAPFGSPAADAPPPAVAAAEVPGSEGGSATGESACHFEGQQRLRGAHAEPYGASPGFGKEPAPPVAVWKGHSFNTISGREAEKFSGKDSGKGDSDFNDSDSDISGDALKKDLINHMQSGLWACTAECKILGHSDRCWSPSCSGPNAHPSPHPPAQMSTFCKSTSLPRDPLRRDNYYQAQLPKTVGLQSVYEKVLHRDYDRTVTLLSPPRPGRLPDLQEIGVPLYQSPPGRYLSPKKGANENV